(19) United States
(12) Patent Application Publication
(10) Pub. No.: US 2025/0289842 A1
(43) Pub. Date: Sep. 18, 2025

(54) Processes for Making NAG-25, a Carbohydrate Targeting Moiety, and its Intermediates

(71) Applicant: AMGEN INC., Thousand Oaks, CA (US)

(72) Inventors: Kyle D. BAUCOM, Ventura, CA (US); Carolyn Michelle COHEN, Somerville, MA (US); Edward HELBLING, Cambridge, MA (US); Heather Claire JOHNSON, Jamaica Plain, MA (US); Sharon E. MICHALAK, Calabasas, CA (US); Philipp C. ROOSEN, Niantic, MI (US); Shea J. O'SULLIVAN, Studio City, CA (US); Amanda STAHL, Agoura Hills, CA (US); Janine K. TOM, Thousand Oaks, CA (US)

(21) Appl. No.: 18/862,498

(22) PCT Filed: May 2, 2023
(86) PCT No.: PCT/US2023/020713

§ 371 Date: Nov. 1, 2024

Related U.S. Application Data
(60) Provisional application No. 63/337,316, filed on May 2, 2022.
(60) Provisional application No. 63/373,823, filed on Aug. 29, 2022.
(60) Provisional application No. 63/385,615, filed on Nov. 30, 2022.

(51) Int. Cl. C07H 15/04; C07C 231/14; C07C 269/06; C07H 1/00; C07H 15/12

(52) U.S. Cl. CPC C07H 15/04; C07C 231/14; C07C 269/06; C07H 1/00; C07H 15/12

(57) ABSTRACT

Provided herein are improved processes for the preparation of a carbohydrate targeting moiety and its intermediates (I).

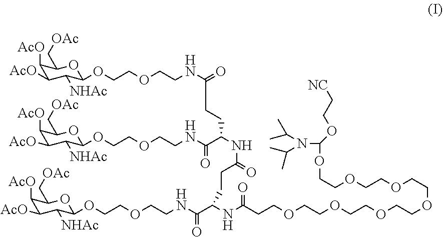 [SMILES: CC(=O)NC1C(OCCOCCNC(=O)CC[C@H](NC(=O)CC[C@H](NC(=O)CCOCCOCCOCCOCCOCCOCCOC(OCCC#N)N(C(C)C)C(C)C)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]

### CROSS-REFERENCE TO RELATED APPLICATION

This application claims the benefit of priority of U.S. Provisional Patent Applications No. 63/337,316, filed May 2, 2022; No. 63/373,823, filed Aug. 29, 2022; and No. 63/385,615, filed Nov. 30, 2022, each of which are incorporated by reference in its entirety.

### TECHNICAL FIELD

The present disclosure relates to improved processes for the preparation of a carbohydrate targeting moiety and its intermediates, and compositions of any of the foregoing. The carbohydrate targeting moiety are useful for conjugation to therapeutic compounds to direct the therapy to the target in vivo.

### BACKGROUND

For certain compounds to have a therapeutic effect (e.g., in vivo) or to be useful for diagnostic purposes, the compounds need to be delivered to a specific location, such as a target cell. Efficient delivery of a compound to a specific location or a target can also reduce or eliminate unintended results (e.g., off-target effects) caused by administration of the compound. A targeting moiety (e.g., carbohydrate targeting moiety) can be linked to such compounds, such as therapeutic compounds (e.g., oligomeric compounds), to direct the therapy to the target in vivo. An example of an oligomeric compound is small interfering RNA (siRNA), which can modulate expression of a target nucleic acid resulting in altered translation of a target nucleic acid to protein. For example, a siRNA can modulate expression of a target gene to inhibit protein translation or expression.

Targeting moieties containing N-acetylgalactosamine (GalNAc or NAG) (e.g., tri-GalNAc or triantennary-GalNAc) facilitate delivery of therapeutic compounds to hepatocytes via binding to the asialoglycoprotein receptor. Exemplary GalNAc-containing targeting moieties, including the compound designated as NAG-25 (or NAG25), for delivering oligomeric compounds to the liver are described in U.S. Pat. No. 10,246,709. U.S. Pat. No. 10,246,709 discloses one method of preparing NAG-25, which has the following structure:

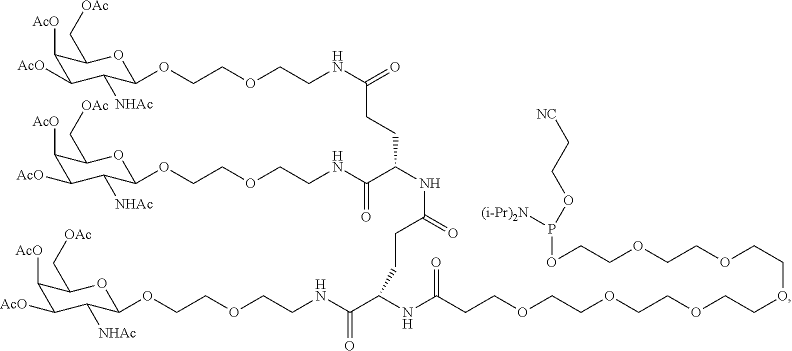

wherein Ac is acetyl (or —(C═O)CH₃) and i-Pr is isopropyl (or 1-methylethyl).

### SUMMARY OF INVENTION

Described herein are processes for preparing NAG-25, which arose from recognition of the need for NAG-25 manufacturing processes that are more efficient, streamlined, environmentally friendly, and/or economical, including processes that result in NAG-25 and its intermediates having increased purities and/or yields. Such processes contain no chromatography steps, minimize or prevent formation of impurities, reduce or reject reactive impurities, and/or improve isolation points and physical properties of intermediates. Therefore, described herein is a process whereby NAG-25 is prepared without the use of column chromatography. Further described herein is a process whereby NAG-25 is prepared using a crystalline form of the Triacid intermediate. Additional new improved processes are further described herein. As a result of the process steps described, isolation of NAG-25 intermediates and yields have been improved, impurities have been reduced, and waste has been reduced. Further described herein are compositions comprising NAG-25, or one of its intermediates (e.g., t-Butyl Core, Triacid or its salts, Alcohol-Z, NAG-Z, NAG-H or its salt, Triol, unprotected Triol, TGZ, TG Amine or its salt, TG PEG, and/or salts of any of the foregoing), with reduced levels of impurities such as the impurities and the percentages of impurities described herein.

Disclosed herein are processes for preparing t-Butyl Core

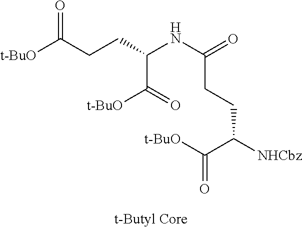 [SMILES: CC(C)(C)OC(=O)CC[C@H](NC(=O)CC[C@H](NC(=O)OCc1ccccc1)C(=O)OC(C)(C)C)C(=O)OC(C)(C)C]

said process comprising reacting GluZ:

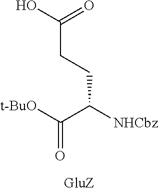 [SMILES: CC(C)(C)OC(=O)[C@H](CCC(=O)O)NC(=O)OCc1ccccc1]

with GluOtBu or salt thereof:

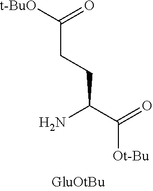 [SMILES: CC(C)(C)OC(=O)CC[C@H](N)C(=O)OC(C)(C)C]

or salt thereof
to produce t-Butyl Core. In some embodiments, the GluOtBu or salt thereof is a GluOtBu hydrochloride salt:

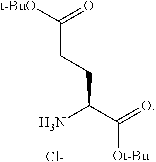 [SMILES: CC(C)(C)OC(=O)CC[C@H](N)C(=O)OC(C)(C)C ; [Cl-]]

In some embodiments, the reaction is performed in the presence of comprising a coupling reagent, a base, and a solvent. In some embodiments, the coupling reagent is EDC/Oxyma, TFFH, PyOxim, CDI, PivCl, T3P, or COMU. In some embodiments, the coupling reagent is T3P or PivCl. In some embodiments, the couple reagent is PivCl. In some embodiments, the base is N-methylmorpholine (NMM). In some embodiments, the solvent is IPAc, MeTHF (also referred to as 2-MeTHF), MIBK, or MTBE. In some embodiments, the solvent is MTBE. In some embodiments, the antisolvent is heptane. In some embodiments, the solvent is MTBE and the antisolvent is heptane. In some embodiments, a first solution comprising GIuZ and NMM in a solvent is added to a second solution comprising the solvent and PivCl. In some embodiments, PivCl is in excess. In some embodiments, GluOtBu is in excess. In some embodiments, conversion to t-Butyl Core is greater than about 90%, based on the amount of GluZ.

Disclosed herein are processes for preparing Triacid:

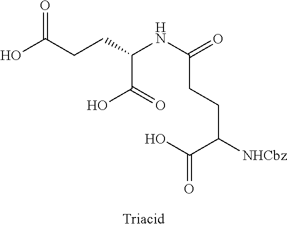 [SMILES: O=C(O)CC[C@H](NC(=O)CCC(NC(=O)OCc1ccccc1)C(=O)O)C(=O)O]

said process comprising reacting t-Butyl core with an acid. In some embodiments, the acid is phosphoric acid (H₃PO₄), TFA, HCl, benzenesulfonic acid, or p-toluenesulfonic acid. In some embodiments, the acid is phosphoric acid (H₃PO₄). In some embodiments, the reaction is conducted in a solvent selected from 2-MeTHF, acetonitrile, THF, DMA, sulfolane, and DME. In some embodiments, the solvent is 2-MeTHF, THF, or acetonitrile. In some embodiments, the solvent is 2-MeTHF or THF. In some embodiments, the solvent is 2-MeTHF. In some embodiments, the Triacid is isolated as a crystalline solid. In some embodiments, the Triacid has greater than or equal to 95% purity as measured by LC. In some embodiments, the solvent further comprises water. In some embodiments, the solvent is a mixture of 2-MeTHF and water. In some embodiments, about 3.0-4.0 vol of 2-MeTHF and about 0.5-2 vol water is used. In some embodiments, the reaction is conducted at a temperature of about 40-60° C. In some embodiments, the reaction is conducted at a temperature of about 45-55° C. In some embodiments, the reaction is conducted at a temperature of about 50° C. In some embodiments, the process further comprising removing phosphoric acid with at least one organic/aqueous wash. In some embodiments, the organic/aqueous wash comprises isopropylacetate (iPAC) as organic layer and ammonium sulfate as aqueous layer. In some embodiments, about 8-18 vol of iPAC and about 3-7 vol 20 wt % of ammonium sulfate are used. In some embodiments, about 10 vol of iPAC and about 5 vol 20 wt % of ammonium sulfate are used. In some embodiments, the process further comprising crystallization of Triacid using acetone/toluene, 2-MeTHF/IPAc/, 2-MeTHF/CPME, acetone/heptane, or MeTHF/acetonitrile. In some embodiments, the process further comprising crystallization of Triacid using acetone/toluene. In some embodiments, the acetone/toluene is in ratio of 2:3, 1:1, 3:2, or 1:2. In some embodiments, the acetone/toluene is in ratio of 2:3, 1:1, or 1:2. In some embodiments, processes produces the Triacid in a crystalline form characterized by an X-ray powder diffractogram having at least a signal at three two-theta values chosen from 7.4±0.2, 9.2±0.2, 21.0±0.2, 14.6±0.2, 18.3±0.2, and 19.3±0.2.

Disclosed herein is a crystalline Form I of Triacid, characterized by an X-ray powder diffractogram having at least a signal at three two-theta values selected from 7.4±0.2, 9.2±0.2, 21.0±0.2, 14.6±0.2, 18.3±0.2, and 19.3±0.2. In some embodiments, the crystalline Form I of Triacid, characterized by an X-ray powder diffractogram having a signal at two-theta values of 7.4±0.2, 9.2±0.2, and 21.0±0.2. In some embodiments, the crystalline Form I of Triacid, characterized by an X-ray powder diffractogram substantially similar to that in FIG. 2. In some embodiments, the crystalline Form II of Triacid, characterized by an X-ray powder diffractogram substantially similar to that in FIG. 3.

Disclosed herein is a process of preparing TGZ

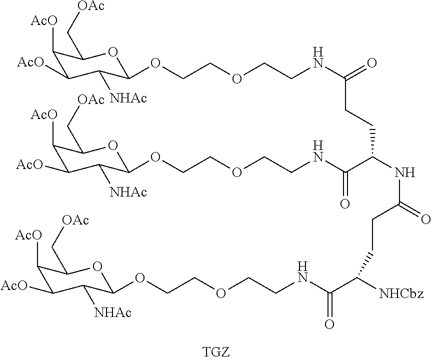 [SMILES: CC(=O)NC1C(OCCOCCNC(=O)CC[C@H](NC(=O)CC[C@H](NC(=O)OCc2ccccc2)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]

said process comprising reacting Triacid with NAG-H:

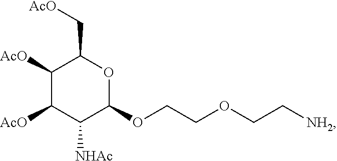 [SMILES: CC(=O)N[C@H]1[C@H](OCCOCCN)O[C@H](COC(C)=O)[C@H](OC(C)=O)[C@@H]1OC(C)=O]

or salt thereof, in the presence of a coupling reagent and a base. In some embodiments, the NAG-H or salt thereof is a TFA salt of NAG-H:

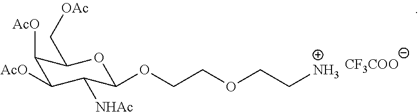 [SMILES: CC(=O)NC1C(OCCOCC[NH3+])OC(COC(C)=O)C(OC(C)=O)C1OC(C)=O ; "CF3COO"]

In some embodiments, the coupling reagent is TBTU, HATU or TCFH. In some embodiments, the coupling reagent is TBTU. In some embodiments, the base is DIPEA, NMI, NMM, or TMP. In some embodiments, the base is NMI. In some embodiments, the process further comprising at least one buffer wash. In some embodiments, the buffer wash is a phosphate buffer. In some embodiments, the buffer wash is about pH 5-7. In some embodiments, the buffer wash is about pH 6. In some embodiments, the process is conducted in a solvent selected from DCM, DMF, MeCN, and DMAc, or combination thereof. In some embodiments, the solvent is DMAc. In some embodiments, the solvent is DCM. In some embodiments, the process further comprising adding an anti-solvent to a solution comprising TGZ and the solvent. In some embodiments, the anti-solvent is an ethereal solvent. In some embodiments, the ethereal solvent is DME, 2-MeTHF, or MTBE. In some embodiments, the solvent is DCM and the anti-solvent is MTBE. In some embodiments, the TGZ is prepared with reduced impurities. In some embodiments, the impurity side product is des-acyl. In some embodiments, the process further comprises precipitation of TGZ from solvent/antisolvent system. In some embodiments, the solvent/antisolvent system is DCM/MTBE. In some embodiments, the TGZ is obtained in at least 95% purity without use of column chromatography. In some embodiments, the NAG-H, or salt thereof, was carried forward as a solution from a prior reaction to react with Triacid without isolation of the NAG-H, or salt thereof, from the prior reaction. In some embodiments, NAG-H, or salt thereof, is prepared in a prior reaction comprising comprising hydrogenation of NAG-Z the presence of a Pd/C catalyst and in DCM or dimethylacetamide (DMAc). In some embodiments, the DCM is about 3 volumes to about 6 volumes. In some embodiments, the DMAc is about 3 volumes to about 5 volumes. In some embodiments, the DMAc is about 4 volumes. In some embodiments, the NAG-H or salt thereof is NAG-H TFA. In some embodiments, the Pd/C catalyst is 5% Pd/C. In some embodiments, NAG-H TFA is prepared in a prior reaction comprising the reagents in the below scheme:

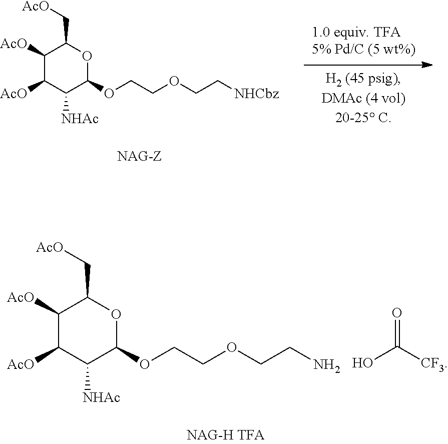 [SMILES: CC(=O)N[C@H]1[C@H](OCCOCCNC(=O)OCc2ccccc2)O[C@H](COC(C)=O)[C@H](OC(C)=O)[C@@H]1OC(C)=O ; CC(=O)N[C@H]1[C@H](OCCOCCN)O[C@H](COC(C)=O)[C@H](OC(C)=O)[C@@H]1OC(C)=O ; O=C(O)C(F)(F)F]

Disclosed herein is a process for preparing Methyl Core:

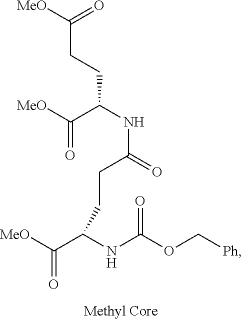 [SMILES: COC(=O)CC[C@H](NC(=O)CC[C@H](NC(=O)OCc1ccccc1)C(=O)OC)C(=O)OC]

said process comprising reacting z-L-Glu-OMe:

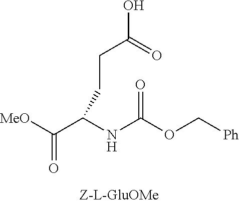 [SMILES: COC(=O)[C@H](CCC(=O)O)NC(=O)OCc1ccccc1]

with L-Glutamic acid dimethyl ester (di(OMe)Glu):

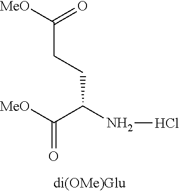 [SMILES: COC(=O)CC[C@H](NCl)C(=O)OC]

to produce Methyl Core.

In some embodiments, the reaction is performed in the presence of a base and a coupling reagent. In some embodiments, the base is added to a z-L-GluOMe solution prior to addition of the coupling reagent and di(OMe)Glu to the reaction. In some embodiments, the coupling reagent is added to the solution of z-L-Glu-OMe and base prior to addition of di(OMe)Glu to the reaction. In some embodiments, the reaction further comprising a solvent. In some embodiments, the base is N-Methylmorpholine (NMM) or di-isopropylethylamine. In some embodiments, the coupling reagent is isobutyl chloroformate (IBCF), TBTU, HATU, EDC, or DCC. In some embodiments, the solvent is THF or MeTHF. In some embodiments, the reaction temperature prior to and during the reaction with coupling reagent is about −20° C. to about −10° C. In some embodiments, the reaction temperature is about −18° C. to about −13° C. In some embodiments, the reaction temperature after addition of di(OMe)Glu is increased to about 5° C. to about 15° C. In some embodiments, the reaction temperature is increased to about 7° C. to about 12° C. In some embodiments, the reaction further comprises an additional charge of coupling reagent, base, and/or z-L-Glu-OMe. In some embodiments, the reaction further comprising washing the reaction with aqueous acid followed by washing the reaction with aqueous base. In some embodiments, the reaction further comprises crystallization by adding anti-solvent. In some embodiments, the process comprises the reagents and conditions in the below scheme:

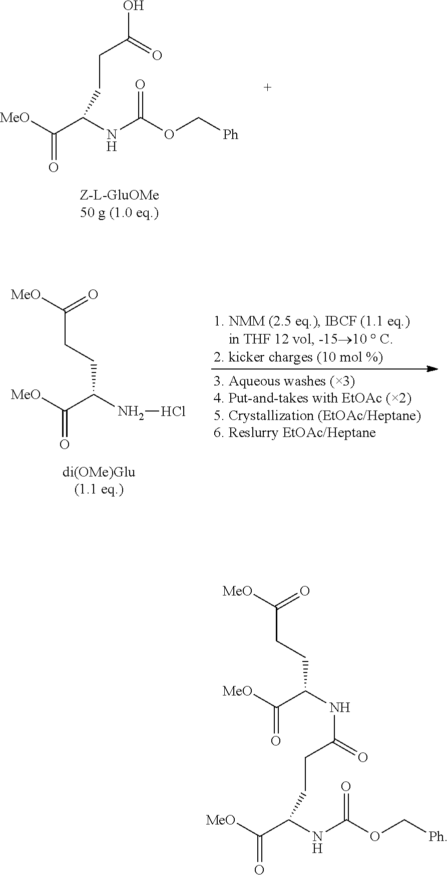 [SMILES: COC(=O)[C@H](CCC(=O)O)NC(=O)OCc1ccccc1 ; COC(=O)CC[C@H](NCl)C(=O)OC ; COC(=O)CC[C@H](NC(=O)CC[C@H](NC(=O)OCc1ccccc1)C(=O)OC)C(=O)OC]

Disclosed herein is a process for preparing Triol:

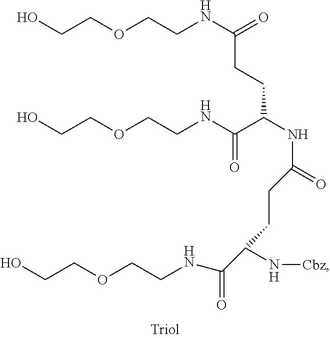

the process comprising reacting Methyl Core with 2-(2-aminoethoxy)ethanol:

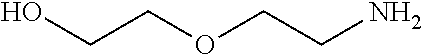

to produce Triol. In some embodiments, the process is performed with neat 2-(2-aminoethoxy)ethanol. In some embodiments, the reaction temperature is at about 25° C. to 35° C. In some embodiments, the reaction temperature is about 30° C. In some embodiments, the reaction further comprising adding an anti-solvent to result in precipitation of the Triol. In some embodiments, the anti-solvent is ethyl acetate, methyl-tert-butyl ether, or iso-propylacetate. In some embodiments, the process further comprises adding a Triol seed crystal. In some embodiments, the process comprises the reagents and conditions in the below scheme:

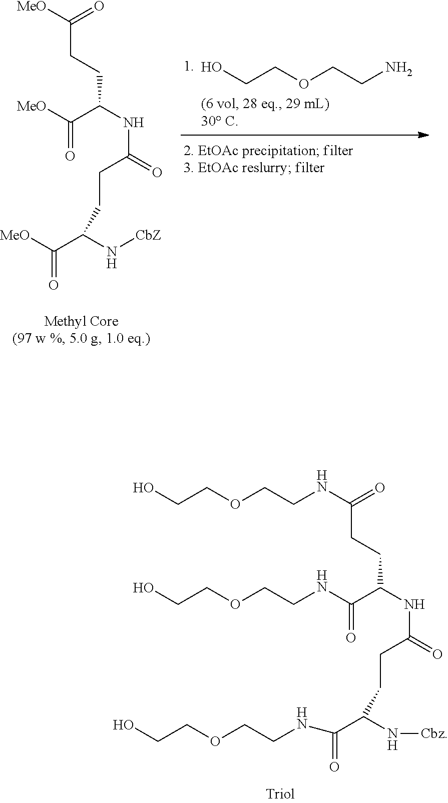

Disclosed is an alternative process for preparing TGZ, the process comprising reacting Triol:

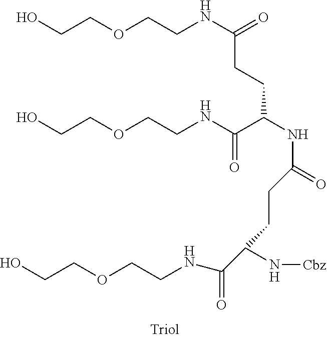

with Beta-D-Galactosamine pentaacetate:

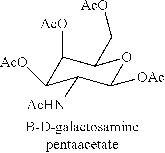 [SMILES: CC(=O)NC1C(OC(C)=O)OC(COC(C)=O)C(OC(C)=O)C1OC(C)=O]

pentaacetate to produce TGZ.

In some embodiments, the Beta-D-Galactosamine pentaacetate is first reacted with a silyl-triflate to produce an oxazoline solution. In some embodiments, the silyl triflate is Trimethylsilyl trifluoromethanesulfonate (TMSOTf) or Triisopropylsilyl Trifluoromethanesulfonate (TIPSOTf). In some embodiments, the reaction further comprises solvent. In some embodiments, the solvent is dichloroethane (DCE) or dichloromethane (DCM). In some embodiments, the reaction temperature is about 35° C. to about 45° C. In some embodiments, the reaction temperature is about 40° C. In some embodiments, the reaction temperature is decreased to about 20° C. to about 28° C. In some embodiments, the reaction temperature is about 23° C. In some embodiments, the Triol is mixed with sodium bicarbonate (NaHCO₃) to produce a slurry mixture of Triol and NaHCO₃ prior to addition of Beta-D-Galactosamine pentaacetate or its oxazoline solution. In some embodiments, the slurry mixture of Triol and NaHCO₃ further comprises a solvent. In some embodiments, the solvent is dichloromethane (DCM), dichloroethane, or acetonitrile. In some embodiments, the oxazoline solution is added to the slurry mixture of triol and NaHCO₃. In some embodiments, the reaction temperature is about 20° C. to about 30° C. In some embodiments, the reaction temperature is about 25° C. In some embodiments, TGZ is further precipitated from a solvent/antisolvent system. In some embodiments, an anti-solvent is added to a solution comprising TGZ and the solvent. In some embodiments, the anti-solvent is dimethoxyethane. In some embodiments, the process comprises the reagents and conditions in the below scheme:

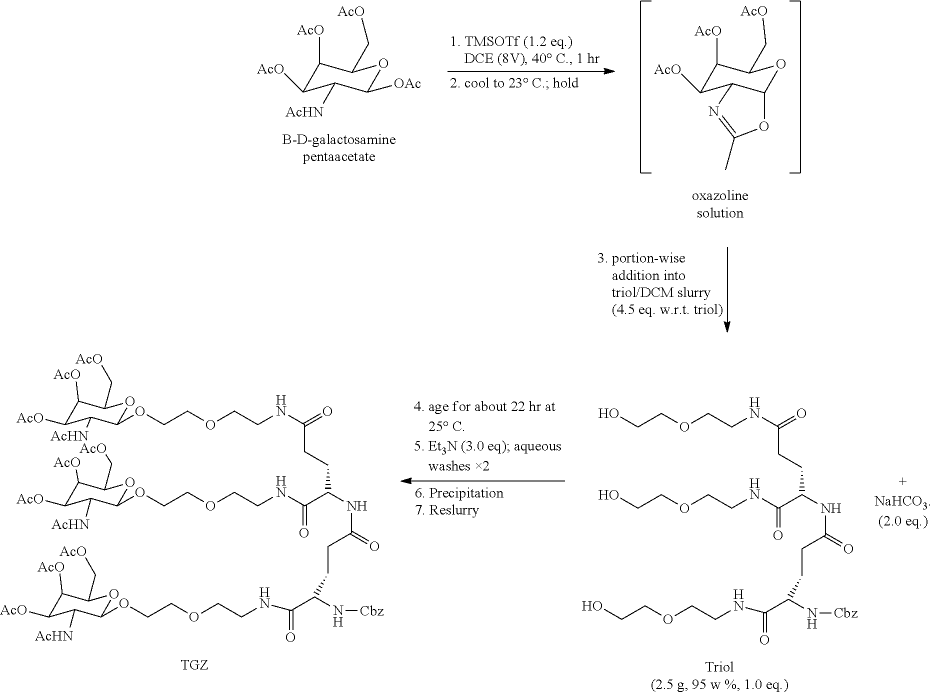

In some embodiments, the reaction further comprising any one of claims for steps A-2 or A-1.

Disclosed herein is a compound

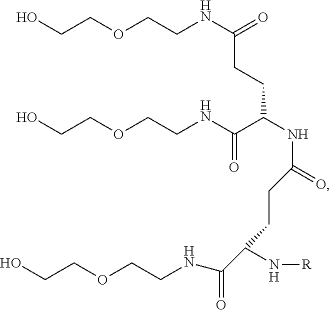

wherein R is H or a Cbz protecting group, which is useful for preparing TGZ as described herein.

Disclosed herein is a process for preparing a NAG-25, said process comprising any one of the embodiments for preparing the Methyl Core or Triol using the alternative processes described. Disclosed herein is are alternative processes for preparing NAG-25, said process comprising a Triol intermediate. In some embodiments, the alternative processes further comprises any one of embodiments for step 5a, step 5b, or step 6. In some embodiments, the alternative processes comprises reacting z-L-Glu-OMe and L-Glutamic acid dimethyl ester to fonn the Methyl Core, and transforming the Methyl Core into NAG-25. In some embodiments, the alternative processes comprises reacting Methyl Core with 2-(2-aminoethoxy)ethanol to form the Triol, and transforming the Triol into NAG-25. In some embodiments, the alternative processes comprises reacting triol with Beta-D-Galactosamine pentaacetate to form TGZ, and transforming the TGZ into NAG-25. In some embodiments, the TGZ is further precipitated from a solvent/antisolvent system as described herein.

Disclosed herein is a process of preparing TG Amine

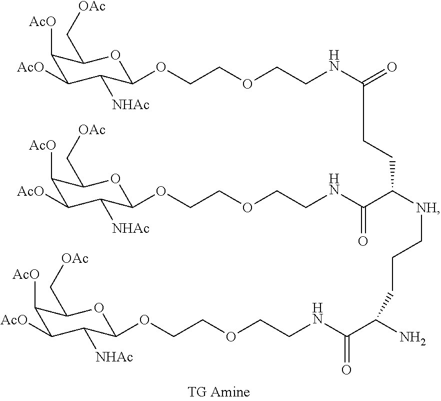 [SMILES: CC(=O)NC1C(OCCOCCNC(=O)CC[C@H](NCCC[C@H](N)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]

or a salt thereof,
said process comprising a high pressure hydrogenolysis for a carboxybenzyl deprotection of TGZ to form the TG Amine or a salt thereof. In some embodiments, the process comprises a palladium source for hydrogenolysis. In some embodiments, the palladium source is palladium on carbon (Pd/C) catalyst. In some embodiments, the palladium source is 5% Pd/C.4d. In some embodiments, the catalyst loading is less than about 10%, 9%, 8%, 7%, 6% Pd/C. In some embodiments, the catalyst used is 5% Pd/C. In some embodiments, the hydrogenolysis is performed in the presence of an acid. In some embodiments, the acid is TFA, oxalic acid, HCl, AcOH, H₃PO₄, citric. In some embodiments, the acid is TFA or oxalic acid. In some embodiments, the acid is TFA. In some embodiments, said hydrogenolysis is performed in a solvent, and the solvent is DCM, IPAc, or MeOH. In some embodiments, the solvent is DCM. In some embodiments, the process reduces formation of des-acyl impurities. In some embodiments, the TG Amine or salt thereof is a TG Amine acid salt. In some embodiments, the TG Amine acid salt is a phosphate, formate, acetate, trifluoroacetate, or oxalate salt. In some embodiments, the TG Amine acid salt is trifluoroacetate or oxalate salt. In some embodiments, the TG Amine acid salt is trifluoroacetate salt. In some embodiments, the process results in high purity of the TG PEG product of Step 5b while reducing the amount of a TG Amine acetamide side product. In some embodiments, the TG Amine or salt thereof comprises less than about 10%, 9%, 8%, 7%, 6%, 5%, 4%, 3%, or 2% acetamide side product. In some embodiments, the TG Amine or salt thereof produced from the improved step 5a process has greater than or equal to about 97%, 98%, or 99% purity as measured by LC. In some embodiments, the TG Amine or salt thereof is telescoped into Step 5b without isolation of the TG Amine or salt thereof. In some embodiments, the process comprises the reagents in the below scheme:

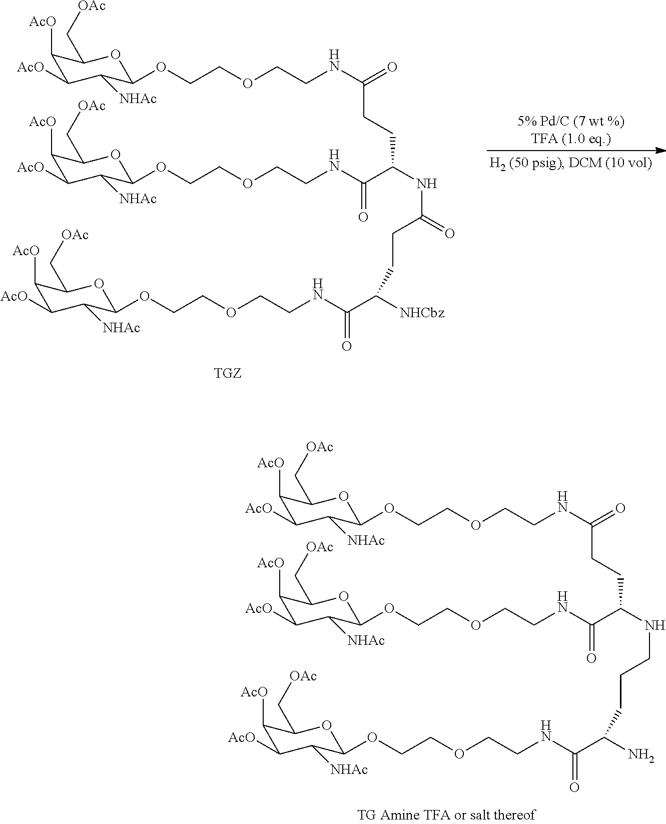 [SMILES: CC(=O)NC1C(OCCOCCNC(=O)CC[C@H](NC(=O)CC[C@H](NC(=O)OCc2ccccc2)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O ; CC(=O)NC1C(OCCOCCNC(=O)CC[C@H](NCCC[C@H](N)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]

Disclosed herein is a process of preparing TG PEG

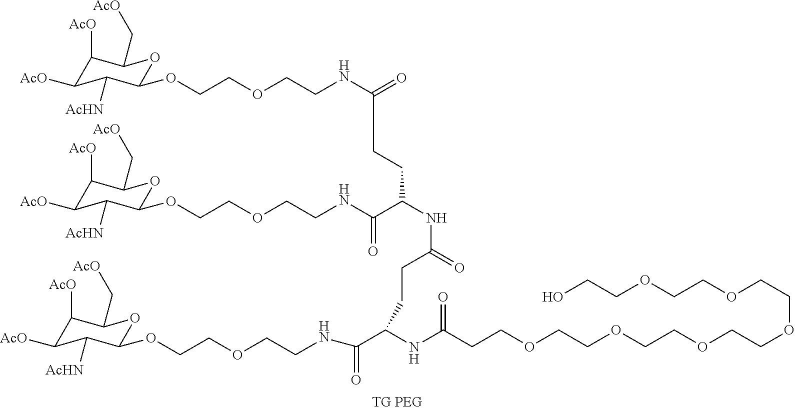

said process comprising treating a solution of TG Amine and PEG acid with TBTU. In some embodiments, the solution further comprises a base. In some embodiments, the base is N,N-Diisopropylethylamine (DIPEA). In some embodiments, the TBTU is added over a period of time of about 30 min to about 1.5 hours. In some embodiments, the TBTU is added over a period of time of about 1-1.5 hour. In some embodiments, the TG PEG is produced in greater than or equal to about 90% purity as measured by LC. In some embodiments, TG PEG dimer impurity is present at the end of reaction at an amount of less than 10% as measured by LC. In some embodiments, the process comprises the reagents in the below scheme:

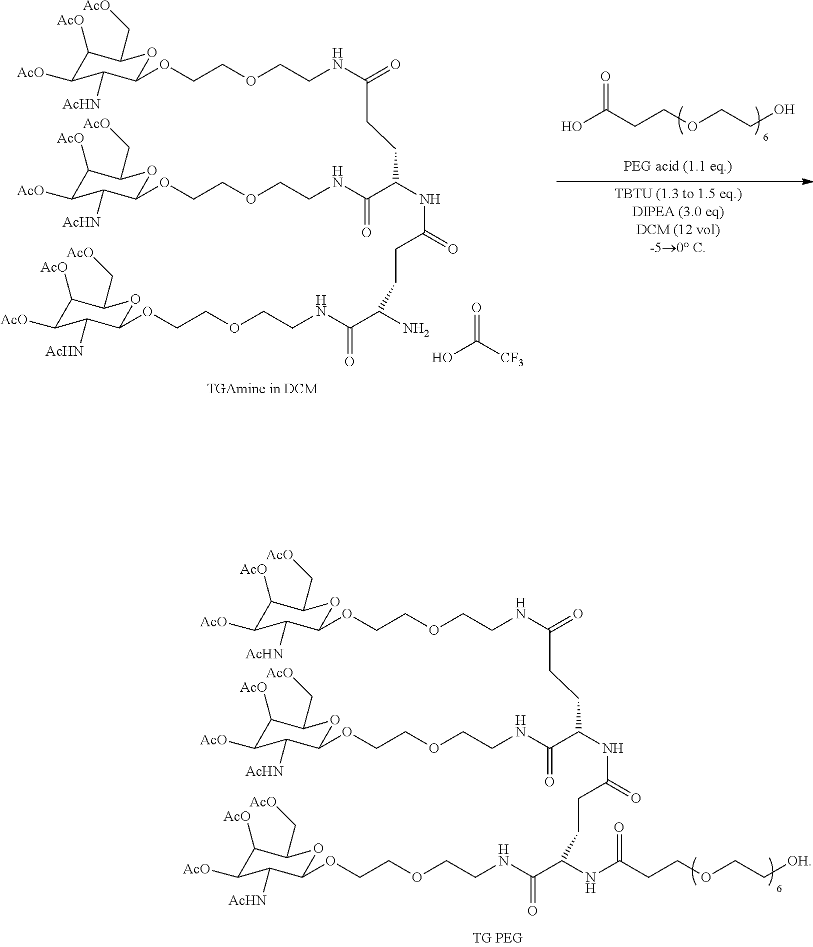

Disclosed herein is a process of preparing NAG-25 comprising treating TG PEG with an activator and a phosphitylating reagent. In some embodiments, the activator is tetrazole, 4,5-dicyanoimidazole (DCI), ETT, or Benzothiotetrazole (BTT). In some embodiments, the activator is tetrazole, DCI, or ETT. In some embodiments, the activator is tetrazole. In some embodiments, the tetrazole is added in about 0.2 eq to 1.2 eq. In some embodiments, the activator is DCI. In some embodiments, the DCI is added in about 0.02 eq to 1.0 eq. In some embodiments, said process is performed in the presence of a base. In some embodiments, the base is NMI. In some embodiments, the phosphitylating reagent is 2-cyanoethyl-N, N, N′, N′-tetraisopropylphosphorodiamidite or 2-Cyanoethyl NN-diisopropylchlorophosphoramidite. In some embodiments, the process further comprises extraction, filtration, precipitation, and drying. In some embodiments, the precipitation step comprises precipitation of NAG-25 from DCM/heptane.

Disclosed herein is a process of preparing a NAG-25:

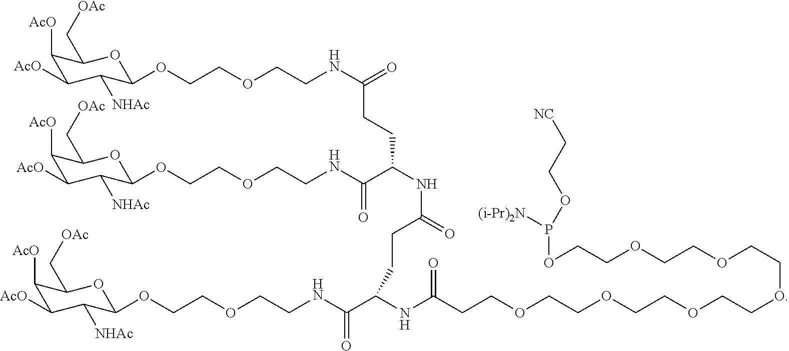

said process comprising any one of the embodiments disclosed herein for steps 1, 2, 3, 4a, 4, 5, or 6. In some embodiments the process comprises preparing t-Butyl Core by any one of the embodiments disclosed herein for preparing t-Butyl Core and transforming the t-Butyl Core into NAG-25. In some embodiments the process comprises preparing Triacid by any one of the embodiments disclosed herein for preparing Triacid and transforming the Triacid into NAG-25. In some embodiments, the process comprises preparing TGZ by any one of the embodiments disclosed herein for preparing TGZ and transforming the TGZ into NAG-25. In some embodiments, the process comprises preparing TG Amine by any one of the embodiments disclosed herein for preparing TG Amine and transforming the TG Amine into NAG-25. In some embodiments, the process comprises preparing TG PEG by any one of the embodiments disclosed herein for TG PEG and transforming the TG PEG into NAG-25.

Disclosed herein is a process of preparing NAG-25:

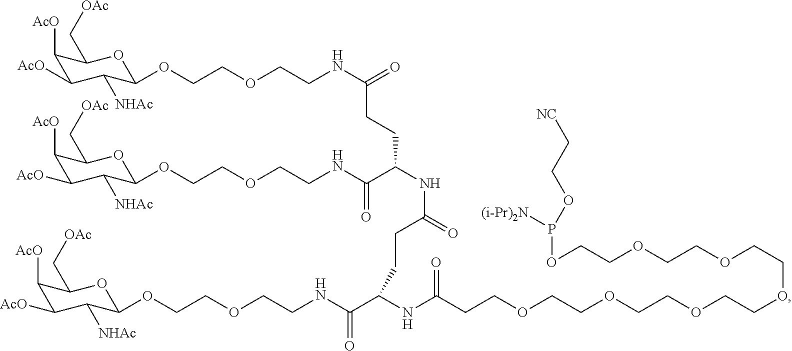

comprising treating a mixture of TG Amine and PEG acid with TBTU to afford TG PEG

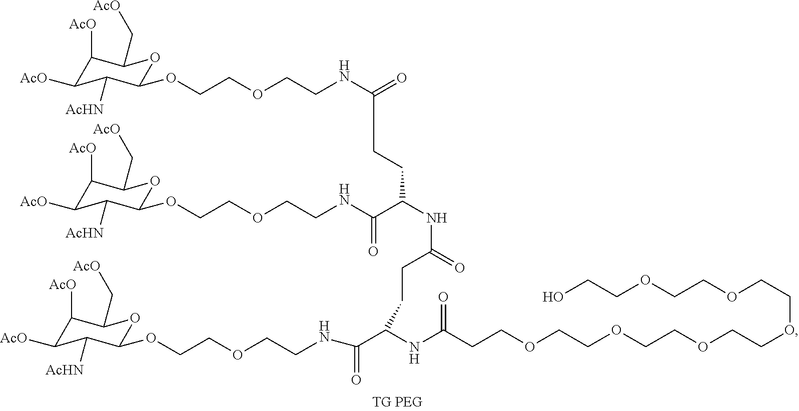

and
transforming TG PEG into NAG-25. Any of the foregoing embodiments and further embodiments described herein related to treating a mixture of TG Amine and PEG acid with TBTU to afford TG PEG would be useful for preparing NAG-25.

Disclosed herein is a process of preparing NAG-25, comprising a high pressure hydrogenolysis for a carboxybenzyl deprotection of TGZ to form the TG Amine:

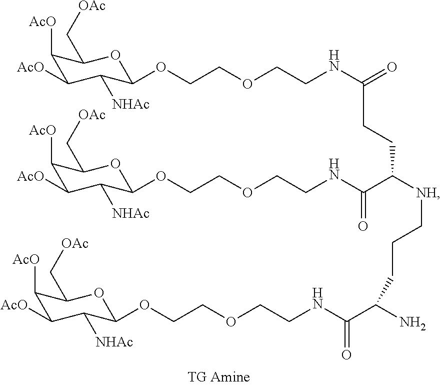 [SMILES: CC(=O)NC1C(OCCOCCNC(=O)CC[C@H](NCCC[C@H](N)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]

or a salt thereof,
and transforming TG Amine, or a salt thereof, into NAG-25. Any of the foregoing embodiments and further embodiments described herein related to a high pressure hydrogenolysis for a carboxybenzyl deprotection of TGZ to form the TG Amine would be useful for preparing NAG-25.

Disclosed herein is a process of preparing NAG-25, comprising reacting Tri acid

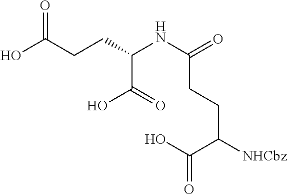 [SMILES: O=C(O)CC[C@H](NC(=O)CCC(NC(=O)OCc1ccccc1)C(=O)O)C(=O)O]

with NAG-H or salt thereof in the presence of a coupling reagent and a base to form TGZ, and transforming TGZ into NAG-25. In some embodiments, TGZ is further precipitated from a solvent/antisolvent system. Any of the foregoing embodiments and further embodiments described herein related to reacting Triacid with NAG-H or salt thereof would be useful for preparing NAG-25.

Disclosed herein is a process of preparing NAG-25, comprising reacting t-Butyl core with an acid to form the Triacid, and transforming the Triacid into NAG-25. In some embodiments, the Triacid is crystalline. In some embodiments, the Triacid is crystalline Form I. Any of the foregoing embodiments and further embodiments described herein related to reacting t-Butyl core with an acid to form the Triacid would be useful for preparing NAG-25.

Disclosed herein is a process of preparing NAG-25, comprising reacting GluZ with GluOtBu or salt thereof to form the t-Butyl core, and transforming the t-Butyl Core into NAG-25. Any of the foregoing embodiments and further embodiments described herein related to processes comprising reacting GluZ with GluOtBu or salt thereof to form the t-Butyl core would be useful for preparing NAG-25.

Disclosed herein is a process of preparing a NAG-25, said process comprising a precipitation of TGZ from solvent/antisolvent system. Any of the foregoing embodiments and further embodiments described herein related to a precipitation of TGZ from solvent/antisolvent system would be useful for preparing NAG-25.

Disclosed herein is a process of preparing a NAG-25, said process comprising a Triacid intermediate that is crystalline. In some embodiments, the crystalline Triacid is crystalline Form I of Triacid, characterized by an X-ray powder diffractogram having at least a signal at three two-theta values chosen from 7.4±0.2, 9.2±0.2, 21.0±0.2, 14.6±0.2, 18.3±0.2, and 19.3±0.2. In some embodiments, the crystalline Triacid is crystalline Form I of Triacid, characterized by an X-ray powder diffractogram having a signal at two-theta values of 7.4±0.2, 9.2±0.2, and 21.0±0.2. In some embodiments, the crystalline Triacid is crystalline Form I of Triacid, characterized by an X-ray powder diffractogram substantially similar to that in FIG. 2. In some embodiments, the crystalline Triacid is crystalline Form II of Triacid, characterized by an X-ray powder diffractogram substantially similar to that in FIG. 3. Any of the foregoing embodiments and further embodiments described herein related to crystalline Triacid would be useful for preparing NAG-25.

Disclosed herein is a process for preparing t-Butyl Core comprising reacting GluZ with GluOtBu or salt thereof to produce t-Butyl Core, wherein the reacting is performed in the presence of a coupling reagent, a base, and a solvent; optionally wherein the a) coupling reagent is EDC/Oxyma, TFFH, PyOxim, CDI, PivCl, T3P, or COMU; or b) base is N-methylmorpholine (NMM); and/or c) solvent is IPAc, MeTHF, MIBK, or MTBE.

Disclosed herein is a process of preparing Triacid comprising reacting t-Butyl core with an acid, wherein the acid is phosphoric acid (H3PO4), TFA, HCl, benzenesulfonic acid, or p-toluenesulfonic acid.

Disclosed herein is a process for preparing NAG-Z comprising reacting Acyl GalNAc with Alcohol-Z to produce NAG-Z, wherein the reaction is performed in the presence of acid.

Disclosed herein is a process of preparing NAG-H, or salt thereof, comprising hydrogenation of NAG-Z in the presence of a Pd/C catalyst and solvent, optionally wherein the solvent is dimethylacetamide (DMAc) or DCM.

Disclosed herein is a process of preparing TGZ comprising reacting Triacid with NAG-H, or a salt thereof, in the presence of a coupling reagent and a base; and precipitating TGZ from a solvent/antisolvent system.

Disclosed herein is a process of preparing TG Amine, or a salt thereof, comprising a high pressure hydrogenolysis of TGZ to form the TG Amine or a salt thereof, wherein said hydrogenolysis is performed in the presence of an acid.

Disclosed herein is a process for preparing TG PEG comprising reacting a solution of TG Amine, or a salt thereof, and PEG acid with a coupling reagent, wherein the coupling reagent is added to a solution of PEG acid and TG Amine or salt thereof over a period of time of about 30 min to about 1.5 hours.

Disclosed herein is a process of preparing NAG-25 comprising reacting TG PEG with an activator and a phosphitylating reagent, wherein the activator is tetrazole, 4,5-dicyanoimidazole (DCI), 5-Ethylthio-1H-Tetrazole (ETT), or Benzothiotetrazole (BTT); and/or wherein the phosphitylating reagent is 2-cyanoethyl-N, N, N′, N′-tetraisopropylphosphorodiamidite or 2-Cyanoethyl N,N-diisopropylchlorophosphoramidite.

Further aspects and advantages will be apparent to those of ordinary skill in the art from a review of the following detailed description, taken in conjunction with the drawings. The description hereafter includes specific embodiments with the understanding that the disclosure is illustrative and is not intended to limit the invention to the specific embodiments described herein.

### BRIEF DESCRIPTION OF THE FIGURES

FIG. 1 shows crystals of Triacid crystalline Form I formed during an acetone/toluene crystallization.

FIG. 2 shows XRPD pattern of Triacid crystalline Form I after toluene/acetone crystallization.

FIG. 3 shows XRPD pattern of Triacid crystalline Form II.

FIG. 4 shows results of Step 5b's TG PEG conversion between 3 solvents (DCM, DMF, MeCN) under different conditions.

FIG. 5 shows enzyme screening results: (A) HPLC results from select reactions in MeCN; (B) HPLC results from select reactions in THF; (C) HPLC results from select reactions in neat conditions.

### DEFINITIONS

As used herein, the terms “including” and “comprising”, and variations such as “include”, “includes”, “comprise”, or “comprises”, are used in an open-ended fashion, and thus should be interpreted to mean “including, but not limited to . . . ”. The terms will also be understood to imply the inclusion of a stated integer or step or group of integers or steps but not the exclusion of any other integer or step or group of integers or steps.

The use of the terms “a,” “an,” “the,” and similar referents in the context of the disclosure herein (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure herein and is not a limitation on the scope of the disclosure herein unless otherwise indicated. No language in the specification should be construed as indicating any non-claimed element as essential to the practice of the disclosure herein.

As used herein, the terms “crystal form,” “crystalline form,” and “Form” interchangeably refer to a crystal structure (or polymorph) having a particular molecular packing arrangement in the crystal lattice. Crystalline forms can be identified and distinguished from each other by one or more characterization techniques including, for example, X-ray powder diffraction (XRPD), single crystal X-ray diffraction, differential scanning calorimetry (DSC), dynamic vapor sorption (DVS), and/or thermogravimetric analysis (TGA). Accordingly, as used herein, the terms “crystalline Form I” and “crystalline Form II” refer to unique crystalline forms that can be identified and distinguished from each other by one or more characterization techniques including, for example, X-ray powder diffraction (XRPD), single crystal X-ray diffraction, differential scanning calorimetry (DSC), dynamic vapor sorption (DVS), and/or thermogravimetric analysis (TGA). In some embodiments, the novel crystalline forms are characterized by an X-ray powder diffractogram having one or more signals at one or more specified degrees two-theta values (° 2θ).

As used herein, the term “XRPD” refers to the analytical characterization method of X-ray powder diffraction. XRPD patterns can be recorded at ambient conditions in transmission or reflection geometry using a diffractometer.

As used herein, the terms “X-ray powder diffractogram,” “X-ray powder diffraction pattern,” “XRPD pattern” interchangeably refer to a pattern plotting signal positions (on the abscissa) versus signal intensities (on the ordinate). For an amorphous material, an X-ray powder diffractogram may include one or more broad signals. For a crystalline material, an X-ray powder diffractogram may include one or more signals, each identified by its angular value as measured in degrees 2θ (° 2θ), depicted on the abscissa of an X-ray powder diffractogram, which may be expressed as “a signal at . . . degrees two-theta,” “signal(s) at [a] two-theta value(s) of . . . ” and/or “at least a signal at . . . two-theta value(s) chosen from . . . ” The term “X-ray powder diffractogram having a signal at . . . two-theta values” as used herein refers to an XRPD pattern that contains signal(s) or peak(s) at the specified position (° 2θ).

A “signal” or “peak” as used herein refers to a point in the XRPD pattern where the intensity as measured in counts is at a local maximum. One of ordinary skill in the art would recognize that one or more signals (or peaks) in an XRPD pattern may overlap and may, for example, not be apparent to the naked eye. Indeed, one of ordinary skill in the art would recognize that some art-recognized methods are capable of and suitable for determining whether a signal exists in a pattern, such as Rietveld refinement.

The repeatability of the angular values is in the range of ±0.2°2θ, i.e., the angular value can be at the recited angular value +0.2 degrees two-theta, the angular value −0.2 degrees two-theta, or any value between those two end points (angular value +0.2 degrees two-theta and angular value −0.2 degrees two-theta).

The terms “signal intensities” and “peak intensities” interchangeably refer to relative signal intensities within a given X-ray powder diffractogram. Factors that can affect the relative signal or peak intensities include sample thickness and preferred orientation (e.g., the crystalline particles are not distributed randomly).

As used herein, an X-ray powder diffractogram is “substantially similar to that in [a particular] Figure” when at least 90%, such as at least 95%, at least 98%, or at least 99%, of the signals in the two diffractograms appear at overlapping positions in degrees two-theta. In determining “substantial similarity,” there may be variation in the intensities and/or signal positions in XRPD diffractograms even for the same crystalline form. Thus, the signal maximum values in XRPD diffractograms (in degrees two-theta (° 2θ) referred to herein) generally mean that value reported ±0.2 degrees 2θ of the reported value, an art-recognized variance.

As used herein, a crystalline form of a compound is “substantially pure” when it accounts for an amount by weight equal to or greater than 90% of the sum of all solid form(s) of the compound in a sample as determined by a method in accordance with the art, such as quantitative XRPD. In some embodiments, the solid form is “substantially pure” when it accounts for an amount by weight equal to or greater than 95% of the sum of all solid form(s) of the compound in a sample. In some embodiments, the solid form is “substantially pure” when it accounts for an amount by weight equal to or greater than 99% of the sum of all solid form(s) of the compound in a sample.

As used herein, the term “solvent” refers to any liquid in which a compound is at least partially soluble (e.g., solubility of product >1 g/L).

As used herein, the term “anti-solvent” refers to any liquid in which a compound is insoluble or at maximum sparingly soluble (e.g., solubility of product <1 mg/mL, <2 mg/mL, <3 mg/mL, or <0.01 mol/L).

As used herein, the term “overall yield” is the cumulative yield of the combination of steps. For example, to get the overall yield for steps 1, 2, and 3, the yield of each step is multiplied together.

As used herein, environmental impact factor (“E-factor”) is a metric for green chemistry and measures the total waste relative to product. High E-factor indicates more waste generation and negative environmental impact.

The terms “about” and “approximately”, when used in connection with a quantity or range, include the value of that quantity or range and includes quantities or ranges that is recognized by one of ordinary skill in the art to provide an effect equivalent to that obtained from the quantity or range. In some embodiments, the term “about” modifies a specified number by + or −10%. In some embodiments, the term “about” modifies a specified number by + or −5%. In some embodiments, the term “about” modifies a specified number by + or −2%. In some embodiments, the term “about” modifies a specified number by + or −1%.

As used herein, when “about” is followed by a series of numbers, it is understood that “about” applies to all the numbers that follow. Similarly, when a unit (e.g., %, hours, equivalents) comes at the end of a series or ranges of numbers, that unit applies to all the series or numbers that came before that unit. For example, “about 3, 4, or 5%” is the same as “about 3%, about 4%, or about 5%”; “about 5-10 equivalents” is the same as “about 5 equivalents to about 10 equivalents”; “about 3-10 hours, 3-6 hours, 4-9 hours” is the same as “about 3-10 hours, about 3-6 hours, about 4-8 hours”; or “about 50-70 minutes or 60 minutes” is the same as “about 50-70 minute or about 60 minutes.”

As used herein, an “oligomeric compound” is a compound comprising at least one oligonucleotide containing about 10-100 nucleotides. In some embodiments, an oligomeric compound has a nucleobase sequence that is at least partially complementary to a coding sequence in an expressed target nucleic acid or target gene within a cell. In some embodiments, the oligomeric compounds, upon delivery to a cell expressing a gene, are able to inhibit the expression of the underlying gene and are referred to herein as “expression-inhibiting oligomeric compounds.” The gene expression can be inhibited in vitro or in vivo. “Oligomeric compounds” include, but are not limited to: oligonucleotides, single-stranded oligonucleotides, single-stranded antisense oligonucleotides, short interfering RNAs (siRNAs), double-strand RNAs (dsRNA), micro RNAs (miRNAs), short hairpin RNAs (shRNA), ribozymes, interfering RNA molecules, and dicer substrates.

As used herein, the term “oligonucleotide” means a polymer of linked nucleosides each of which can be independently modified or unmodified.

As used herein, the term “oligonucleotide” means a polymer of linked nucleosides each of which can be independently modified or unmodified.

As used herein, the term “single-stranded oligonucleotide” means a single-stranded oligomeric compound having a sequence at least partially complementary to a target mRNA that is capable of hybridizing to a target mRNA through hydrogen bonding or Watson Crick base pairing under mammalian physiological conditions (or comparable conditions in vitro). In some embodiments, a single-stranded oligonucleotide is a single stranded antisense oligonucleotide.

As used herein, an “RNAi construct” refers to an agent comprising an RNA or RNA-like (e.g., chemically modified RNA) oligonucleotide molecule that is capable of degrading or inhibiting translation of messenger RNA (mRNA) transcripts of a target gene in a sequence specific manner. As used herein, RNAi constructs may operate through the RNA interference mechanism (i.e., inducing RNA interference through interaction with the RNA interference pathway machinery (RNA-induced silencing complex or RISC) of mammalian cells), or by any alternative mechanism(s) or pathway(s). RNAi constructs include, but are not limited to: single-stranded oligonucleotides, single-stranded antisense oligonucleotides, short interfering RNAs (siRNAs), double-strand RNAs (dsRNA), micro RNAs (miRNAs), short hairpin RNAs (shRNA), and dicer substrates. The RNAi constructs suitable for conjugation to the targeting ligands (e.g. NAG-25) described herein are comprised of an oligonucleotide having a strand that is at least partially complementary to the mRNA being targeted. In some embodiments, the RNAi constructs are double-stranded, and are comprised of an antisense strand and a sense strand that is at least partially complementary to the antisense strand. RNAi constructs may be comprised of modified nucleotides and/or one or more non-phosphodiester linkages. In some embodiments, the RNAi constructs suitable for conjugation to the targeting ligands (e.g. NAG-25) described herein are single-stranded.

As used herein, “LCAP”, which stands for “liquid chromatography area percent”, means the percentage of the peak area of the compound of interest in relation to total area of peaks.

As used herein, a “salt” of a compound described herein can be prepared, for example, by reacting the compound in its free base form with a suitable organic or inorganic acid, and optionally isolating the salt thus formed. Nonlimiting examples of suitable salts for any one or more of the compounds described herein include hydrobromide, hydrochloride, sulfate, bisulfate, sulfonate, camphorsulfonate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like.

### DETAILED DESCRIPTION

The following discussion is directed to various exemplary embodiments of the disclosed processes. However, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and that the scope of this disclosure, including the claims, is not limited to that embodiment.

### Processes for NAG-25 and its Intermediates

Provided herein are processes for the preparation of NAG-25, which is a carbohydrate targeting moiety, and NAG-25's intermediates: t-Butyl Core, Triacid, Alcohol-Z, NAG-Z, NAG-H, Triol, Methyl Core, TGZ, TG Amine, TG PEG, and salts of any of the foregoing. The processes described herein provide NAG-25 and the foregoing intermediates with reduced levels of impurity side products, reduced number of unit operations and/or streamlined work up/isolation used in the multi-step NAG-25 process. Further provided herein are processes for isolating and/or crystallizing certain intermediates such as the Triacid.

The processes, intermediates, and products described herein are useful for preparing therapeutic compounds, such as siRNA molecules that are conjugated to NAG-25, a carbohydrate targeting moiety with the below structure:

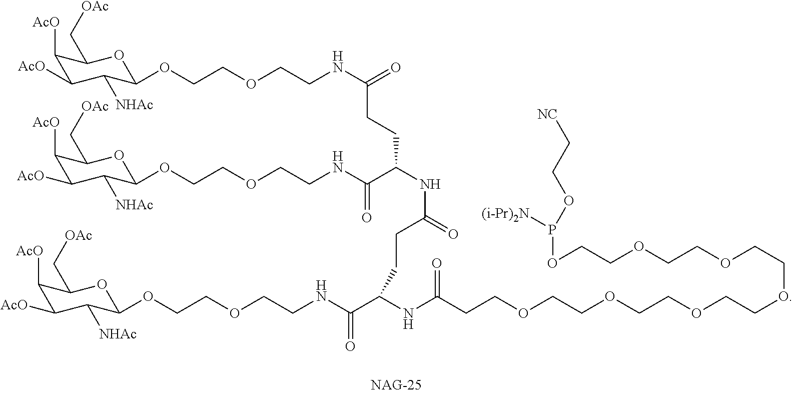

As further described herein, NAG-25 can be produced starting from any one of the steps described herein. As will be further described, the compounds 1-tert-Butyl N-Carbobenzoxy-L-glutamate (GluZ) and L-Glutamic acid di-tert-butyl ester (GluOtBu) or salt thereof (e.g., GluOtBu hydrochloride salt) are coupled to obtain t-Butyl Core (step 1), which can be followed by the tri-ester deprotection to produce Triacid (step 2). Triacid can then be coupled with three units of NAG-H or salt thereof (e.g., NAG-H TFA) resulting in TGZ (step 4b). NAG-H TFA can be synthesized by reductive deprotection of the Cbz group from NAG-Z (step 4a). NAG-Z can be synthesized by direct glycosylation of Alcohol-Z with Acyl GalNAc (step 3b). Alcohol-Z can optionally be prepared from amino protection of aminoalcohol as described herein (step 3a). TG Amine or salt thereof can be obtained by reductive deprotection of the Cbz group from TGZ (step 5a). TG Amine or salt thereof can be coupled with PEG acid to yield TG PEG (step 5b). Finally, NAG-25 can be synthesized by phosphoramidite formation from TG PEG (step 6).

As further described herein, NAG-25 can be produced using an alternative process to produce TGZ. The alternative processes utilize a different protecting group strategy. The alternative process comprises amide coupling (Step A-1), aminolysis (Step A-2), and glycosylation (Step A-3). In this alternative process, TGZ is prepared using a Triol intermediate as further described herein. Following TGZ isolation using the alternative processes described herein, TGZ can be converted to NAG-25 using Steps 4, 5, and/or 6 as described herein.

The NAG-25 process comprises one or more steps that reduced, or are free or substantially free of, one or more impurities and/or side products, which are described herein.

Described herein are compositions comprising NAG-25, or its one of its intermediates, with reduced levels of one or more impurities, which are further described herein. The disclosed processes provide NAG-25 in high chemical purity. In various embodiments, the chemical purity of NAG-25 prepared according to the disclosed processes is 90% or more, as determined by liquid chromatography.

For example, in various embodiments, the chemical purity of NAG-25 is at least 91%, 92%, 93%, 94%, 95%, 96%, 97%, 98%, 99%. 99.5%, or 99.9%, as determined by liquid chromatography.

In contrast to the prior process described in U.S. Pat. No. 10,246,709, the processes disclosed herein comprise improvements at each step, such as the option at the end of Step 4b (which produces TGZ) to avoid column chromatography, which can be costly, wasteful, and time intensive for large scale synthesis, and a new crystal form of Triacid isolated at the end of Step 2, which helps to reject or remove impurities in a more controlled manner.

### Processes for Preparing NAG-25

Disclosed herein is a process of preparing NAG-25 comprising step 1, step 2, and/or step 4b processes. The combination of steps 1, 2, and 3 processes described herein results in an overall yield improvement (e.g., improved from about 47% to about 67%) and/or reduction in the environmental impact factor (E-factor) (e.g., reduced from about 1000 to about 540).

Disclosed herein is a process of preparing NAG-25 comprising a step 1 process that achieves close to complete reaction conversion, optionally without additional amounts (or charges) of one or more reagents or starting materials (also known as a kicker charge).

Disclosed herein is a process of preparing NAG-25 comprising a step 2 process that results in a new crystalline Triacid (e.g., Triacid crystal form I).

Disclosed herein is a process of preparing NAG-25 comprising a step 4b process that isolates and purifies TGZ using precipitation from a solvent/antisolvent system, without the use of column chromatography.

Disclosed herein is a process of preparing NAG-25 comprising a step 3b process that results in NAG-Z, which is one of the starting materials for NAG-25. The step 3b process can be performed without isolation of an oxazoline intermediate and has decreased impurities and improved yields. Further disclosed herein is a step 3a process that results in Alcohol-Z, used as a starting material for NAG-Z, with a more streamlined process (fewer unit operations), improved yields and/or decreased impurities.

Disclosed herein is a process of preparing NAG-25 comprising Step A-1, Step A-2, and/or Step A-3 to produce TGZ and its alternative intermediates (Methyl Core and Triol), which are further described herein. Further disclosed is a Triol intermediate (benzyl ((10S,15S)-1,22-dihydroxy-10-((2-(2-hydroxyethoxy)ethyl)carbamoyl)-7,12,16-trioxo-3,20-dioxa-6,11,17-triazadocosan-15-yl)carbamate) that is useful for preparing TGZ. Provided herein is a composition comprising the Triol intermediate and the unprotected Triol. Disclosed herein is a process of preparing NAG-25 wherein the process comprises the Triol intermediate.

Disclosed herein is a process of preparing NAG-25 comprising step 5a, step 5b, and/or step 6 processes. The combination of steps 4, 5, and 6 processes described herein results in streamlined unit operations, reduced solvent volumes, and/or improved purity. Improvements described herein include improved isolation points and physical properties of intermediates and reduction or rejection of reactive impurities. The improved steps 4-6 processes described herein result in reduced unit operations and solvent volumes.

Disclosed herein is a process of preparing NAG-25 comprising a step 5b process in which a coupling reagent (e.g., TBTU) is added over a period of about 1-1.5 hr.

Disclosed herein is a process of preparing NAG-25 comprising any one of step 1, step 2, step 4a, step 3b, step 3a, step 4b, step 5a, step 5b, step 6, and/or embodiments thereof, which are further described herein.

Disclosed herein is a process of preparing NAG-25 comprising any one of step A-1, step A-2, step A-3, step 5a, step 5b, step 6, and/or embodiments thereof, which are further described herein.

Overall, the improved process of preparing NAG-25 disclosed herein comprises improved steps 1-6, which improved the overall NAG-25 yield (e.g., increased by about 6%) and reduced the E-factor (e.g., about 24% reduction in E-factor). The process improvements described herein allows NAG-25 to be more efficiently manufactured on a larger scale.

Step 1: t-Butyl Core

Disclosed herein are processes for preparing t-Butyl Core (step 1 processes). The processes comprise coupling of 1-tert-Butyl N-Carbobenzoxy-L-glutamate (GluZ) and L-Glutamic acid di-tert-butyl ester (GluOtBu) to obtain t-Butyl Core:

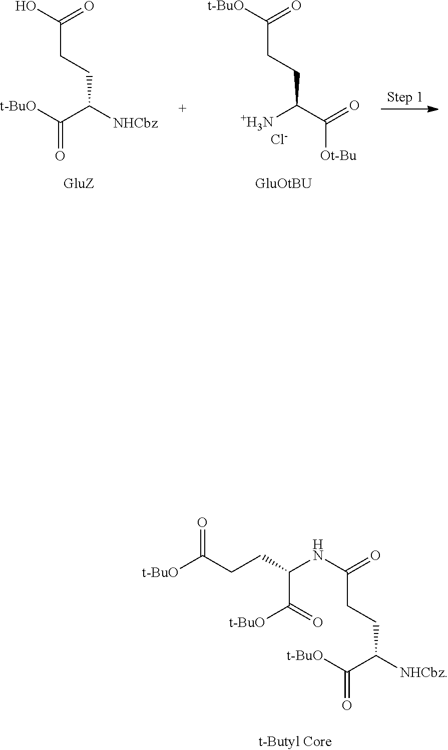 [SMILES: CC(C)(C)OC(=O)[C@H](CCC(=O)O)NC(=O)OCc1ccccc1 ; CC(C)(C)OC(=O)CC[C@H]([NH3+])C(=O)OC(C)(C)C ; [Cl-] ; CC(C)(C)OC(=O)CC[C@H](NC(=O)CC[C@H](NC(=O)OCc1ccccc1)C(=O)OC(C)(C)C)C(=O)OC(C)(C)C]

A prior step 1 process had challenges which included: reaction stalling, which required additional reagent charges (kicker charge) to assist towards full conversion; and crystallization from hexanes, which may not be environmentally friendly. To address the challenges, experiments were performed to investigate the following: alternative coupling reagents that would achieve closer to full conversion without a kicker charge, which was used in a prior process; the addition mode of reagents to increase conversion to t-Butyl Core; and methods to streamline step 1 processes and the crystallization of t-Butyl Core (e.g., by removing a solvent swap that was used in a prior step 1 process).

Disclosed herein is a step 1 process that produces t-Butyl Core, providing higher reaction conversion and/or higher isolated purity while utilizing with a single charge of reagents. The improved step 1 processes uses different coupling reagent, solvent, order of addition, and amount of reagents, resulting in improved reaction conversion to the t-Butyl Core and improved purity of the t-Butyl Core. Step 1 also includes reverse addition of reagents, which (without being bound by theory) is believed to facilitate formation of a mixed anhydride intermediate over the symmetric anhydride intermediate. Furthermore, crystallization of the t-Butyl Core product became more streamlined by switching reaction solvent (from THF to e.g., MTBE), removing the solvent swap step going into crystallization, and reducing solvent volumes for crystallization.

In one aspect of step 1, the process for preparing the t-Butyl Core:

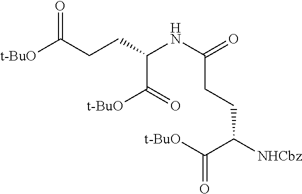 [SMILES: CC(C)(C)OC(=O)CC[C@H](NC(=O)CC[C@H](NC(=O)OCc1ccccc1)C(=O)OC(C)(C)C)C(=O)OC(C)(C)C]

comprises reacting GluZ:

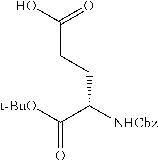 [SMILES: CC(C)(C)OC(=O)[C@H](CCC(=O)O)NC(=O)OCc1ccccc1]

with GluOtBu:

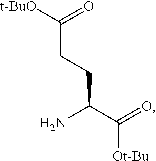 [SMILES: CC(C)(C)OC(=O)CC[C@H](N)C(=O)OC(C)(C)C]

or a salt thereof, to produce the t-Butyl core. In some embodiments, GluOtBu or a salt thereof is used in excess. In some embodiments, GluOtBu or a salt thereof is a GluOtBu salt. In some embodiments, the GluOtBu salt is a hydrochloride salt. In some embodiments of step 1, the process comprises the use of a coupling reagent, a base, and a solvent.

In some embodiments, the coupling reagent is 1-ethyl-3-(3-dimethvlaminopropyl)carbodiimide hydrochloride (EDC)/ethyl cyanohydroxyiminoacetate (Oxyma) (EDC/Oxyma), tetramethylfluoroformamidinium hexafluorophosphate (TFFH), [ethylcyano(hydroxyimino)acetato-O²]tri-1-pyrrolidinylphosphonium hexafluorophosphate (PyOxim), 1,1′-Carbonyldiimidazole (CDI), trimethylacetyl chloride (PivCl), 1-propanephosphonic anhydride solution (T3P), or (1-Cyano-2-ethoxy-2-oxoethylidenaminooxy)dimethylamino-morpholino-carbenium hexafluorophosphate (COMU). In some embodiments, the couple reagent is T3P or PivCl. In some embodiments, the couple reagent is PivCl. In some embodiments, PivCl is added in excess. In some embodiments, the base is N-methylmorpholine (NMM). In some embodiments, the coupling reagent is T3P or PivCl, and the base is NMM. In some embodiments, the coupling reagent is PivCl and the base is NMM.

In some embodiments, the solvent is isopropyl acetate (IPAc), 2-methyltetrahydrofuran (MeTHF), methyl isobutyl ketone (MIBK), or tert-Butyl methyl ether (also known as methyl tert-butyl ether or MTBE). In some embodiments, the solvent is MeTHF or MTBE. In some embodiments, the solvent is MTBE. In some embodiments, an antisolvent is used to induce crystallization of the product (t-Butyl Core). In some embodiments, the antisolvent is heptane. In some embodiments, the antisolvent is 1:2 ratio of IPA:water. In some embodiments, the solvent is MTBE and the antisolvent is heptane.

Step 1 process can use either normal addition or inverse addition of the GluZ to coupling reagent. Under normal addition, a solution of coupling reagent in solvent was added to a solution of GluZ, base, and solvent. In some embodiments, step 1 process comprises normal addition, wherein a solution of coupling reagent in solvent is added to a solution of GluZ and base in solvent. Under inverse addition, a solution of GluZ, base, and solvent was added to a solution of coupling reagent in solvent. Without being bound by theory, inverse addition forms the mixed anhydride that helps to reduce formation of a GluZ symmetric anhydride. In some embodiments, step 1 process comprises inverse addition, wherein a solution of GluZ and base in solvent is added to another solution of coupling reagent in solvent.

A solution of GluZ and base in solvent is prepared and added to a solution of PivCl in solvent. In some embodiments, the solvent is MTBE and a solution of GluZ and NMM in MTBE is added to a solution of MTBE and PivCl. This addition of Gluz and NMM occurs over a time period of about at least 50 minutes, 50-70 minutes or 60 minutes. Following about another 20-40 minutes of stirring, GluOtBu is added in small amounts about every 10-20 minutes and the reaction proceeds for about 20-40 (e.g., 30 minutes) at 0° C. The reaction is monitored (e.g., with LC) until an appropriate conversion is achieved.

In some embodiments, each of PivCl and GluOtBu or salt thereof is independently used in about 1 eq to about 1.5 eq. In some embodiments, the PivCl used is about 1.1, 1.2, or 1.3 eq and the GluOtBu or a salt thereof used is about 1.2, 1.3, or 1.4 eq. In some embodiments, the PivCl used is about 1.2 eq and the GluOtBu or a salt thereof used is about 1.3 eq. The equivalents (“eq” or “equiv” herein) of PivCl and GluOtBu or salt thereof are relative to GluZ.

In some embodiments, NMM used is about 3.0 to about 3.5 eq. In some embodiments, NMM used is about 3.3 to about 3.7 eq. In some embodiments, NMM used is at least about 3.0 eq. In some embodiments, NMM used is at least about 3.5 eq. In some embodiments, NMM used is about 3.5 eq.

In some embodiments, PivCl is about 1.1-1.4 eq, GluOtBu is about 1.1-1.5 eq, and NMM is about 3.3-3.7 eq. In some embodiments, PivCl is about 1.2 eq, GluOtBu is about 1.3 eq, and NMM is about 3.5 eq. The equivalents are relative to GluZ.

In some embodiments, the step 1 process for preparing the t-Butyl Core comprises reacting GluZ with GluOtBu or a salt thereof in the presence of PivCl, NMM, and MTBE to produce the t-Butyl Core.

In some embodiments, the step 1 process for preparing the t-Butyl Core comprises reacting GluZ with GluOtBu or a salt thereof in the presence of PivCl (about 1.1-1.4 eq), NMM (at least 3.0 eq), and MTBE (about 8-12 vol) to produce the t-Butyl Core.

In some embodiments, a step 1 process comprises:
a) combining a first solution of GluZ, base, and solvent with a second solution of coupling reagent and solvent to form a third solution of GluZ, base, solvent, and coupling reagent; and
b) adding GluOtBu or salt thereof to the third solution of GluZ, base, solvent, and coupling reagent.

In some embodiments, a step 1 process comprises:
a) combining a first solution of GluZ, NMM, and MTBE with a second solution of PivCl and MTBE to form a third solution of GluZ, NMM, MTBE, PivCl; and
b) adding GluOtBu or salt thereof to the third solution of GluZ, NMM, MTBE, PivCl.

In some embodiments, GluOtBu or salt thereof is a GluOtBu hydrochloride salt.

The improved step 1 process described herein result in less impurities, such as the Piv-Glu-OH and the tripeptide impurity, which are shown below.

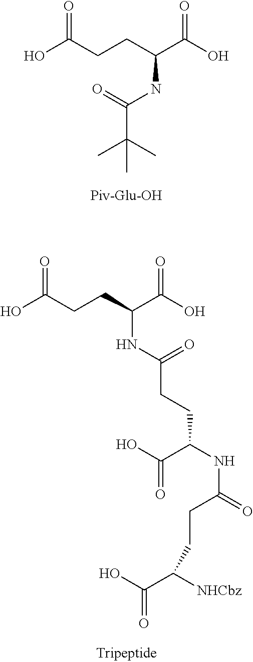

In some embodiments, step 1 produces in t-Butyl Core in greater than about 85% or about 90% yield. In some embodiments, step 1 produces in t-Butyl Core in greater than about 91, 92, 93, 94, 95, 96, or 97% yield.

In some embodiments, step 1 produces t-Butyl Core with greater than about 95% purity as measured by liquid chromatography (LC). In some embodiments, the purity of t-Butyl Core is greater than about 96, 97, 98, or 99%, as measured by LC.

In some embodiments, a step 1 process for preparing the t-Butyl Core is shown in the below scheme:

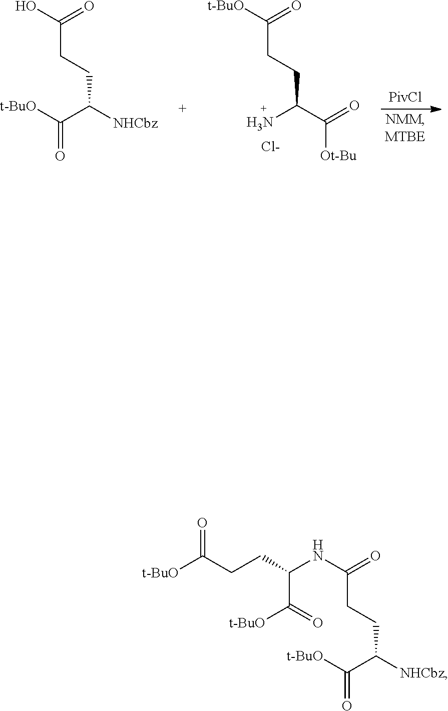 [SMILES: CC(C)(C)OC(=O)[C@H](CCC(=O)O)NC(=O)OCc1ccccc1 ; CC(C)(C)OC(=O)CC[C@H](N)C(=O)OC(C)(C)C ; [Cl-] ; "NMM,MTBE" ; "PivCl" ; CC(C)(C)OC(=O)CC[C@H](NC(=O)CC[C@H](NC(=O)OCc1ccccc1)C(=O)OC(C)(C)C)C(=O)OC(C)(C)C]

wherein GluZ is reacted with GluOtBu HCl in the presence of PivCl, base, MTBE. In some embodiments of the scheme above, PivCl is about 1.2 eq, NMM is about 3.5 eq, MTBE is about 10 vol. In some embodiments of the scheme above, step 1 is performed at about 0° C. In some embodiments, the reaction comprises inverse addition of solution of GluZ and NMM in MTBE to a solution of PivCl in MTBE.

In some embodiments, one, two or three other types of alkyl groups (e.g., methyl, ethyl, propyl, and/or butyl) can replace one, two, or three of the t-butyl groups of t-Butyl Core, GluZ, and GluOtBu.

In some embodiments of step 1, the process further comprises work-up (e.g., extractive washes), distillation, crystallization, filtration, rinsing, and/or drying.

In some embodiments, the extractive work-up step comprises: adding a solution of hydrochloric acid (e.g., about 0.3-0.7 M or 0.5M HCl), agitation followed by phase separation, adding a solution of sodium carbonate (e.g., about 0.3-0.7M or 0.5M sodium carbonate), agitation followed by phase separation, adding brine solution (e.g., 4-6 wt % or 5 wt % brine solution), agitation followed by layer separation. The organic layer can be taken forward to the crystallization or held at 20° C. overnight, if needed.

Further provided herein is a crystallization method for t-Butyl core, comprising adding an antisolvent to a solution of t-Butyl core in solvent. In some embodiments, a seed of t-Butyl core is also added (e.g., added before, after, or with the antisolvent). In some embodiments, the antisolvent is added over a period of time of at least about 2, 3, 4, or 5 hours; or about 2-5, 2-4, 3-5, 3-4, or 2.5-3.5 hours; or about 3, 3.5, or 4 hours. In some embodiments, the antisolvent is added until the fmnal amount is about 30, 40, 50, 60, 70, 80% of antisolvent. In some embodiments, the antisolvent is added to a solution of t-Butyl core in solvent that is held at about 30-40, 35-45, or 33-37° C. In some embodiments, after the antisolvent has been added, the solution temperature is lowered to about 15-25, 18-22, or 20° C. In some of the above embodiments, the solvent is MTBE and the antisolvent is heptane.

### Step 2: Triacid or a Salt Thereof

Disclosed herein are processes for preparing Triacid or a salt thereof (step 2 processes). The processes comprise triester deprotection of t-Butyl Core to produce Triacid or a salt thereof:

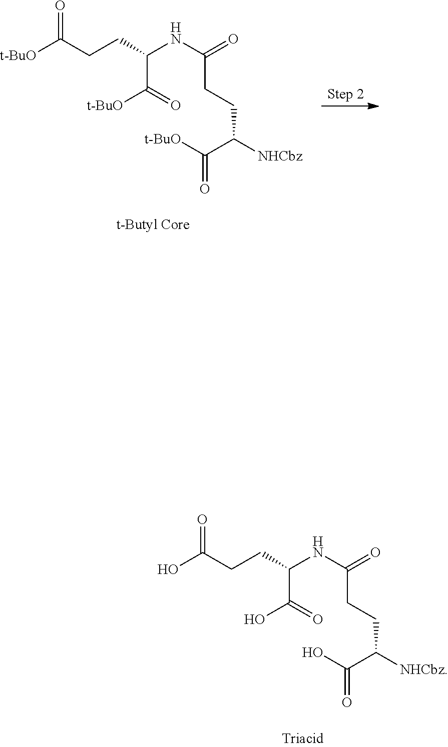 [SMILES: CC(C)(C)OC(=O)CC[C@H](NC(=O)CC[C@H](NC(=O)OCc1ccccc1)C(=O)OC(C)(C)C)C(=O)OC(C)(C)C ; O=C(O)CC[C@H](NC(=O)CC[C@H](NC(=O)OCc1ccccc1)C(=O)O)C(=O)O]

In a prior step 2 process, formic acid was used to deprotect three tert-butyl groups of the t-Butyl Core. Following completion of the reaction, formic acid was removed following a series of solvent swap/distillation operations and the Triacid was isolated as an oil.

To improve on a prior step 2 process, experiments were performed to investigate the following: isolation of solid triacid that enables a purity upgrade; more efficient work-up procedure (e.g., extractive wash); and developing reaction conditions leading to higher purity of Triacid or salt thereof, including use of alternative acids in the reaction.

Disclosed herein are improved step 2 processes that results in high purity Triacid or a salt thereof. Examples of Triacid salts include phosphate, sodium, and calcium salts. In one aspect of step 2, the process for preparing the Triacid, or salt thereof, comprises reacting t-Butyl core with an acid as the deprotection reagent. The reaction can proceed in the presence of solvent or solvent mixed with water. When present with the solvent, the water acts as a tert-butyl scavenger and efficiently produces the Triacid from tert-Butyl Core in high yield.

In some embodiments, the acid is phosphoric acid (H₃PO₄), TFA, HCl, benzenesulfonic acid, or p-toluenesulfonic acid. In some embodiments, the acid is phosphoric acid (H₃PO₄), TFA, or HCl. In some embodiments, the acid is phosphoric acid (H₃PO₄). In some embodiments, the step 2 reaction proceeds in a solvent selected from 2-MeTHF, acetonitrile, THF, DMA, sulfolane, and DME. In some embodiments, the solvent is 2-MeTHF, THF, or acetonitrile. In some embodiments, the solvent is 2-MeTHF or THF. In some embodiments, the solvent is 2-MeTHF. In some embodiments, the solvent is mixed with water. In some embodiments, solvent is 2-MeTHF and is mixed with water. In some embodiments, the solvent is about 3.0-4.0 volumes of 2-MeTHF and is mixed with about 0.3-1, 0.4-0.6, or 0.5-2 volumes of water. In some embodiments, the solvent is about 3.0-4.0 volumes of 2-MeTHF and is mixed with about 0.4-0.6 volumes of water. In some embodiments, the improved step 2 utilizes phosphoric acid (H₃PO₄) as the acid and 2-MeTHF as the solvent mixed with water. In some embodiments, the improved step 2 utilizes phosphoric acid (H₃PO₄) as the acid and 2-MeTHF as the solvent mixed with water, wherein phosphoric acid is present at about 3 vol, 2-MeTHF is present at about 3.5 vol and water is about 0.5 vol.

In some embodiments, reaction is at a temperature of about 40-60° C. In some embodiments, the temperature is about 45-55° C. In some embodiments, the temperature is about 50° C. In some embodiments, the reaction proceeds for about 3-10 hours, 3-6 hours, 4-9 hours, 4-7 hours, or 4-5 hours.

In some embodiments, a step 2 process of preparing Triacid, or a salt thereof, comprises:
a) combining t-Butyl Core in solvent with acid;
b) adding water; and
c) heating the reaction to a certain temperature. In some embodiments, the solvent is 2-MeTHF, the acid is H₃PO₄ and the temperature is about 50° C.

In some embodiments, a step 2 process for preparing the Triacid, or a salt thereof, is shown in the below scheme:

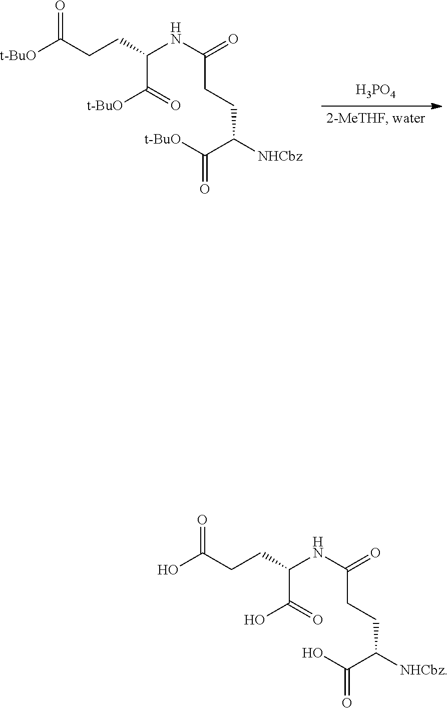 [SMILES: CC(C)(C)OC(=O)CC[C@H](NC(=O)CC[C@H](NC(=O)OCc1ccccc1)C(=O)OC(C)(C)C)C(=O)OC(C)(C)C ; "H3PO4" ; "2-MeTHF, water" ; O=C(O)CC[C@H](NC(=O)CC[C@H](NC(=O)OCc1ccccc1)C(=O)O)C(=O)O]

In some embodiments, a step 2 process for preparing the Triacid, or a salt thereof, is shown in the below scheme:

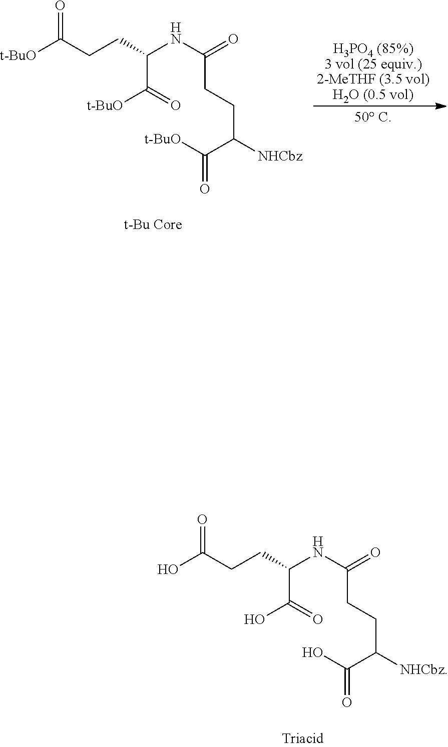 [SMILES: CC(C)(C)OC(=O)CC[C@H](NC(=O)CCC(NC(=O)OCc1ccccc1)C(=O)OC(C)(C)C)C(=O)OC(C)(C)C ; "50° C." ; "H3PO4 (85%)3 vol (25 equiv.)2-MeTHF (3.5 vol)H2O (0.5 vol)" ; O=C(O)CC[C@H](NC(=O)CCC(NC(=O)OCc1ccccc1)C(=O)O)C(=O)O]

The reaction is complete when the amount of diacid is less than 3% as measured by LC. In some embodiments of step 2, after the reaction is finished, the process further comprises work-up (e.g., extractive washes), distillation, crystallization, filtration, and/or drying.

In some embodiments, the acid (e.g., phosphoric acid) is efficiently removed by aqueous workup, which avoids a lengthy formic acid distillation sequence. In some embodiments, step 2 further comprises removing phosphoric acid with at least one organic/aqueous wash. In some embodiments of this aqueous workup step, the reaction mixture is diluted with isopropylacetate and washed with 20 wt % ammonium sulfate aqueous solution. In some embodiments of the aqueous workup step, the reaction mixture is diluted with 2-methyltetrahydrofuran and/or washed with 20 wt % ammonium sulfate aqueous solution. Then, the organic layer is washed with water.

In some embodiments, the organic/aqueous wash comprises isopropylacetate (iPAc) as organic layer and ammonium sulfate as aqueous layer. In some embodiments, about 8-18 vol of iPAc and about 3-8 vol 20 wt % of ammonium sulfate are used. In some embodiments, about 9-11 vol of iPAc and about 4-6 vol 20 wt % of ammonium sulfate are used. In some embodiments, about 10 vol of iPAc and about 5 vol 20 wt % of ammonium sulfate are used.

In some embodiments, the organic/aqueous wash comprises 2-Methyltetrahydrofuran (MeTHF) as organic layer and ammonium sulfate as aqueous layer. In some embodiments, about 6-18 vol of MeTHF and about 3-20 vol 20 wt % of ammonium sulfate are used. In some embodiments, about 8-10 vol of MeTHF and about 12-18 vol 20 wt % of ammonium sulfate are used. In some embodiments, about 9 vol of MeTHF and about 15 vol 20 wt % of ammonium sulfate are used. In some embodiments, the wash volumes listed herein can be divided into 2-4 (e.g., 3) separate smaller wash steps. Following the washes, an optional water wash (e.g., 1, 2, or 3 volumes) can be used.

Additionally, in some embodiments, a solvent/antisolvent crystallization system can be used to provide the final product as high purity crystalline material. In some embodiments, the solvent/antisolvent is acetone/toluene, 2-MeTHF/IPAc, 2-MeTHF/CPME, acetone/heptane, or MeTHF/acetonitrile. In some embodiments, the solvent is a combination of one or more of any of the solvents listed. In some embodiments, the solvent is a combination of MeTHF and acetone. In some embodiments, the solvent is a combination of MeTHF and acetone and the antisolvent is toluene. In some embodiments, the solvent/antisolvent is acetone/toluene. In some embodiments, the solvent is about 2-4 vol of MeTHF with 3-5 vol acetone and the toluene is about 13 vol. In some embodiments, the solvent is about 3 vol of MeTHF with 4 vol acetone and the toluene is about 13 vol. In some embodiments, toluene is in ratio of 2:3, 1:1, 3:2, or 1:2. In some embodiments, the acetone/toluene is in ratio of 2:3, 1:1, or 1:2. In some embodiments, the solvent/antisolvent is acetone/toluene. In some embodiments, the acetone/toluene is in ratio of 2:3, 1:1, 3:2, or 1:2. In some embodiments, the acetone/toluene is in ratio of 2:3, 1:1, or 1:2. In some embodiments, the toluene is about 40, 50, 60, 70% or about 40-70, 40-50, 45-55, 50-60, 55-65, or 60-70% of the total volume of solvent/antisolvent system, wherein the rest of the volume percentage is one or more solvents. In some embodiments, the one or more solvents is acetone or MeTHF combined with toluene.

In some embodiments, the Triacid, or salt thereof, is isolated as a crystalline solid. In some embodiments, the crystalline Triacid is not a salt.

In some embodiments, the Triacid, or salt thereof, is greater than or equal to about 95%, 96%, 97%, 98%, or 99% purity, as measured by LC. In some embodiments, the Triacid, or salt thereof, is greater than or equal to about 95% purity as measured by LC. In some embodiments, the process of preparing Triacid, or salt thereof, from t-Butyl Core comprises crystallization of Triacid, or salt thereof, using a solvent/antisolvent system that is acetone/toluene, MeTHF/IPAc, MeTHF/CPME, acetone/heptane, or MeTHF/acetonitrile. In some embodiments, the process of preparing Triacid, or salt thereof, from t-Butyl Core comprises crystallization of Triacid, or salt thereof, with acetone/toluene. In some embodiments, crystallization comprises seeding an acetone/toluene ratio of about 3:2, 1:1, or 5:6 and charging further toluene until the final ratio of acetone/toluene is about 2:3, 1:2, or 1:3. The seeding can be done with a Triacid seed (e.g., 1 wt %). In some embodiments, the final volume percentage of toluene in the acetone/toluene mixture is about 40-70% toluene. In some embodiments, the final volume percentage of toluene in the acetone/toluene mixture is about 55-70% toluene. Any of the foregoing embodiments are applicable to Triacid (i.e., the free Triacid that is not a salt).

In some embodiments, any one of the step 2 embodiments described herein results in a crystalline Triacid characterized by an X-ray powder diffractogram as further described in the below embodiments.

Disclosed herein are crystal forms of Triacid. In some embodiments, step 2 process produces the Triacid crystalline Form I. In some embodiments, step 2 process produces the Triacid crystalline Form II.

In some embodiments, the Triacid crystalline solid is crystal Form I. In some embodiments, the Triacid crystalline Form I is in substantially pure form. In some embodiments, the Triacid crystalline Form I is characterized by an X-ray powder diffractogram generated by an X-ray powder diffraction analysis with an incident beam of Cu Kα radiation.

In some embodiments, Triacid crystalline Form I is characterized by an X-ray powder diffractogram having a signal at 7.4±0.2 degrees two-theta. In some embodiments, Triacid crystalline Form I is characterized by an X-ray powder diffractogram having a signal at 9.2±0.2 degrees two-theta.

In some embodiments, Triacid crystalline Form I is characterized by an X-ray powder diffractogram having a signal at 21.0±0.2 degrees two-theta. In some embodiments, Triacid crystalline Form I is characterized by an X-ray powder diffractogram having a signal at 10.2±0.2 degrees two-theta. In some embodiments, Triacid crystalline Form I is characterized by an X-ray powder diffractogram having a signal at 14.6±0.2 degrees two-theta. In some embodiments, Triacid crystalline Form I is characterized by an X-ray powder diffractogram having a signal at 18.3±0.2 degrees two-theta. In some embodiments, Triacid crystalline Form I is characterized by an X-ray powder diffractogram having a signal at 19.3±0.2 degrees two-theta. In some embodiments, Triacid crystalline Form I is characterized by an X-ray powder diffractogram having a signal at 22.9±0.2 degrees two-theta. In some embodiments, Triacid crystalline Form I is characterized by an X-ray powder diffractogram having a signal at 11.0±0.2 degrees two-theta.

In some embodiments, Triacid crystalline Form I is characterized by an X-ray powder diffractogram having a signal at 11.3±0.2 degrees two-theta.

In some embodiments, Triacid crystalline Form I is characterized by an X-ray powder diffractogram having a signal at two-theta values of 7.4±0.2, 9.2±0.2, and 21.0±0.2. In some embodiments, Triacid crystalline Fonn I is optionally further characterized by an X-ray powder diffractogram having an additional signal at two-theta values of 10.2±0.2, 14.6±0.2, 18.3±0.2, 19.3±0.2, and 22.9±0.2. In some embodiments, Triacid crystalline Form I is optionally further characterized by an X-ray powder diffractogram having an additional signal at two-theta value of 11.0±0.2 or 11.3±0.2.

In some embodiments, Triacid crystalline Form I is characterized by an X-ray powder diffractogram having a signal at two-theta values of 7.4±0.2, 9.2±0.2, 21.0±0.2, 10.2±0.2, 14.6±0.2, 18.3±0.2, 19.3±0.2, and 22.9±0.2. In some embodiments, Triacid crystalline Form I is characterized by an X-ray powder diffractogram having at least a signal at seven two-theta values selected from 7.4 0.2, 9.2±0.2, 21.0±0.2, 10.2±0.2, 14.6±0.2, 18.3±0.2, 19.3±0.2, and 22.9±0.2. In some embodiments, Triacid crystalline Form I is characterized by an X-ray powder diffractogram having at least a signal at six two-theta values selected from 7.4±0.2, 9.2±0.2, 21.0±0.2, 10.2±0.2, 14.6±0.2, 18.3±0.2, 19.3±0.2, and 22.9±0.2. In some embodiments, Triacid crystalline Form I is characterized by an X-ray powder diffractogram having at least a signal at five two-theta values selected from 7.4±0.2, 9.2±0.2, 21.0±0.2, 10.2±0.2, 14.6±0.2, 18.3±0.2, 19.3±0.2, and 22.9±0.2. In some embodiments, Triacid crystalline Form I is characterized by an X-ray powder diffractogram having at least a signal at four two-theta values selected from 7.4±0.2, 9.2±0.2, 21.0±0.2, 10.2±0.2, 14.6±0.2, 18.3±0.2, 19.3±0.2, and 22.9±0.2. In some embodiments, Triacid crystalline Form I is characterized by an X-ray powder diffractogram having at least a signal at three two-theta values selected from 7.4±0.2, 9.2±0.2, 21.0±0.2, 10.2±0.2, 14.6±0.2, 18.3±0.2, 19.3±0.2, and 22.9±0.2. In some embodiments, Triacid crystalline Form I is characterized by an X-ray powder diffractogram having at least a signal at two two-theta values selected from 7.4±0.2, 9.2±0.2, 21.0±0.2, 10.2±0.2, 14.6±0.2, 18.3±0.2, 19.3±0.2, and 22.9±0.2. In some embodiments, Triacid crystalline Form I is characterized by an X-ray powder diffractogram having at least a signal at one two-theta values selected from 7.4±0.2, 9.2±0.2, 21.0±0.2, 10.2±0.2, 14.6±0.2, 18.3±0.2, 19.3±0.2, and 22.9±0.2.

In some embodiments, Triacid crystalline Form I is characterized by an X-ray powder diffractogram having a signal at two-theta values of 7.4±0.2, 9.2±0.2, 21.0±0.2, 10.2±0.2, 11.0±0.2, 14.6±0.2, 18.3±0.2, 19.3±0.2, and 22.9±0.2. In some embodiments, Triacid crystalline Form I is characterized by an X-ray powder diffractogram having at least a signal at seven two-theta values selected from 7.4±0.2, 9.2±0.2, 21.0±0.2, 10.2±0.2, 11.0±0.2, 14.6±0.2, 18.3±0.2, 19.3±0.2, and 22.9±0.2. In some embodiments, Triacid crystalline Form I is characterized by an X-ray powder diffractogram having at least a signal at six two-theta values selected from 7.4±0.2, 9.2±0.2, 21.0±0.2, 10.2±0.2, 11.0±0.2, 14.6±0.2, 18.3±0.2, 19.3±0.2, and 22.9±0.2. In some embodiments, Triacid crystalline Form I is characterized by an X-ray powder diffractogram having at least a signal at five two-theta values selected from 7.4±0.2, 9.2±0.2, 21.0±0.2, 10.2±0.2, 11.0±0.2, 14.6±0.2, 18.3±0.2, 19.3±0.2, and 22.9±0.2. In some embodiments, Triacid crystalline Form I is characterized by an X-ray powder diffractogram having at least a signal at four two-theta values selected from 7.4±0.2, 9.2±0.2, 21.0±0.2, 10.2±0.2, 11.0±0.2, 14.6±0.2, 18.3±0.2, 19.3±0.2, and 22.9±0.2. In some embodiments, Triacid crystalline Form I is characterized by an X-ray powder diffractogram having at least a signal at three two-theta values selected from 7.4±0.2, 9.2±0.2, 21.0±0.2, 10.2±0.2, 11.0±0.2, 14.6±0.2, 18.3±0.2, 19.3±0.2, and 22.9±0.2. In some embodiments, Triacid crystalline Form I is characterized by an X-ray powder diffractogram having at least a signal at two two-theta values selected from 7.4±0.2, 9.2±0.2, 21.0±0.2, 10.2±0.2, 11.0±0.2, 14.6±0.2, 18.3±0.2, 19.3±0.2, and 22.9±0.2. In some embodiments, Triacid crystalline Form I is characterized by an X-ray powder diffractogram having at least a signal at one two-theta values selected from 7.4±0.2, 9.2±0.2, 21.0±0.2, 10.2±0.2, 11.0±0.2, 14.6±0.2, 18.3±0.2, 19.3±0.2, and 22.9±0.2.

In some embodiments, Triacid crystalline Form I is characterized by an X-ray powder diffractogram having a signal at two-theta values of 7.4±0.2, 9.2±0.2, 21.0±0.2, 10.2±0.2, 11.3±0.2, 14.6±0.2, 18.3±0.2, 19.3±0.2, and 22.9±0.2. In some embodiments, Triacid crystalline Form I is characterized by an X-ray powder diffractogram having at least a signal at seven two-theta values selected from 7.4±0.2, 9.2±0.2, 21.0±0.2, 10.2±0.2, 11.3±0.2, 14.6±0.2, 18.3±0.2, 19.3±0.2, and 22.9f 0.2. In some embodiments, Triacid crystalline Form I is characterized by an X-ray powder diffractogram having at least a signal at six two-theta values selected from 7.4±0.2, 9.2±0.2, 21.0±0.2, 10.2±0.2, 11.3±0.2, 14.6±0.2, 18.3±0.2, 19.3±0.2, and 22.9±0.2. In some embodiments, Triacid crystalline Form I is characterized by an X-ray powder diffractogram having at least a signal at five two-theta values selected from 7.4±0.2, 9.2±0.2, 21.0±0.2, 10.2±0.2, 11.3±0.2, 14.6±0.2, 18.3±0.2, 19.3±0.2, and 22.9±0.2. In some embodiments, Triacid crystalline Form I is characterized by an X-ray powder diffractogram having at least a signal at four two-theta values selected from 7.4±0.2, 9.2±0.2, 21.0±0.2, 10.2±0.2, 11.3±0.2, 14.6±0.2, 18.3±0.2, 19.3±0.2, and 22.9±0.2. In some embodiments, Triacid crystalline Form I is characterized by an X-ray powder diffractogram having at least a signal at three two-theta values selected from 7.4±0.2, 9.2±0.2, 21.0±0.2, 10.2±0.2, 11.3±0.2, 14.6±0.2, 18.3±0.2, 19.3±0.2, and 22.9±0.2. In some embodiments, Triacid crystalline Form I is characterized by an X-ray powder diffractogram having at least a signal at two two-theta values selected from 7.4±0.2, 9.2±0.2, 21.0±0.2, 10.2±0.2, 11.3±0.2, 14.6±0.2, 18.3±0.2, 19.3±0.2, and 22.9±0.2. In some embodiments, Triacid crystalline Form I is characterized by an X-ray powder diffractogram having at least a signal at one two-theta values selected from 7.4±0.2, 9.2±0.2, 21.0±0.2, 10.2±0.2, 11.3±0.2, 14.6±0.2, 18.3±0.2, 19.3±0.2, and 22.9±0.2.

In some embodiments, Triacid crystalline Form I is characterized by an X-ray powder diffractogram having at least a signal at three two-theta values selected from 7.4±0.2, 9.2±0.2, 21.0 0.2, 14.6±0.2, 18.3±0.2, and 19.3±0.2. In some embodiments, Triacid crystalline Form I is characterized by an X-ray powder diffractogram having at least a signal at two two-theta values selected from 7.4±0.2, 9.2±0.2, and 21.0±0.2. In some embodiments, Triacid crystalline Form I is characterized by an X-ray powder diffractogram having a signal at two-theta values of 7.4±0.2, 9.2±0.2, 21.0±0.2, 14.6±0.2, 18.3±0.2, and 19.3±0.2.

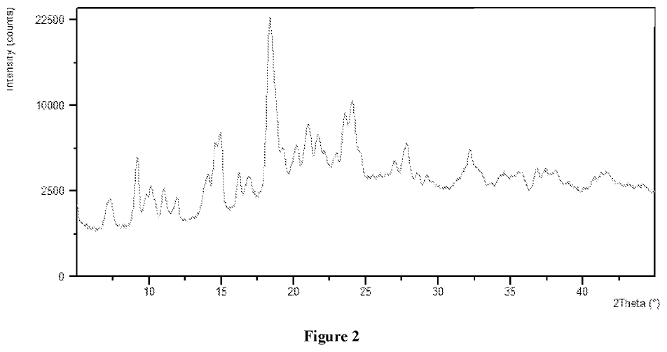

In some embodiments, Triacid crystalline Form I is characterized by an X-ray powder diffractogram substantially similar to that in FIG. 2.

In some embodiments, the Triacid crystalline solid is crystal Form II. In some embodiments, the Triacid crystalline Form II is in substantially pure form. In some embodiments, the Triacid crystalline Form II is characterized by an X-ray powder diffractogram generated by an X-ray powder diffraction analysis with an incident beam of Cu Kα radiation.

In some embodiments, Triacid crystalline Form II is characterized by an X-ray powder diffractogram having a signal at 7.8±0.2 degrees two-theta. In some embodiments, Triacid crystalline Form II is characterized by an X-ray powder diffractogram having a signal at 9.7±0.2 degrees two-theta.

In some embodiments, Triacid crystalline Form II is characterized by an X-ray powder diffractogram having a signal at 15.7±0.2 degrees two-theta. In some embodiments, Triacid crystalline Form II is characterized by an X-ray powder diffractogram having a signal at 13.3±0.2 degrees two-theta. In some embodiments, Triacid crystalline Form II is characterized by an X-ray powder diffractogram having a signal at 17.1±0.2 degrees two-theta. In some embodiments, Triacid crystalline Form II is characterized by an X-ray powder diffractogram having a signal at 18.9±0.2 degrees two-theta. In some embodiments, Triacid crystalline Form II is characterized by an X-ray powder diffractogram having a signal at 20.2±0.2 degrees two-theta. In some embodiments, Triacid crystalline Form II is characterized by an X-ray powder diffractogram having a signal at 21.0±0.2 degrees two-theta.

In some embodiments, Triacid crystalline Form II is characterized by an X-ray powder diffractogram having a signal at two-theta values of 7.8±0.2, 9.7±0.2, and 15.7±0.2. In some embodiments, Triacid crystalline Form II is optionally further characterized by an X-ray powder diffractogram having an additional signal at two-theta values of 13.3±0.2, 17.1±0.2, and/or 18.9±0.2.

In some embodiments, Triacid crystalline Form II is optionally further characterized by an X-ray powder diffractogram having an additional signal at two-theta value of 20.2±0.2 and/or 21.0±0.2.

In some embodiments, Triacid crystalline Form IT is characterized by an X-ray powder diffractogram having a signal at two-theta values of 7.8±0.2, 9.7±0.2, 13.3±0.2, 15.7±0.2, 17.1±0.2, 18.9±0.2, 20.2±0.2 and 21.0±0.2. In some embodiments, Triacid crystalline Form II is characterized by an X-ray powder diffractogram having at least a signal at seven two-theta values selected from 7.8 0.2, 9.7±0.2, 13.3±0.2, 15.7±0.2, 17.1±0.2, 18.9±0.2, 20.2±0.2 and 21.0±0.2. In some embodiments, Triacid crystalline Form II is characterized by an X-ray powder diffractogram having at least a signal at six two-theta values selected from 7.8 0.2, 9.7±0.2, 13.3±0.2, 15.7±0.2, 17.1±0.2, 18.9±0.2, 20.2±0.2 and 21.0±0.2. In some embodiments, Triacid crystalline Form II is characterized by an X-ray powder diffractogram having at least a signal at five two-theta values selected from 7.8±0.2, 9.7±0.2, 13.3±0.2, 15.7±0.2, 17.1±0.2, 18.9±0.2, 20.2±0.2 and 21.0±0.2. In some embodiments, Triacid crystalline Form II is characterized by an X-ray powder diffractogram having at least a signal at four two-theta values selected from 7.8±0.2, 9.7±0.2, 13.3±0.2, 15.7±0.2, 17.1±0.2, 18.9±0.2, 20.2±0.2 and 21.0±0.2. In some embodiments, Triacid crystalline Form ii is characterized by an X-ray powder diffractogram having at least a signal at three two-theta values selected from 7.8±0.2, 9.7±0.2, 13.3±0.2, 15.7±0.2, 17.1±0.2, 18.9±0.2, 20.2±0.2 and 21.0±0.2. In some embodiments, Triacid crystalline Form II is characterized by an X-ray powder diffractogram having at least a signal at two two-theta values selected from 7.8±0.2, 9.7±0.2, 13.3±0.2, 15.7±0.2, 17.1±0.2, 18.9±0.2, 20.2±0.2 and 21.0±0.2. In some embodiments, Triacid crystalline Form II is characterized by an X-ray powder diffractogram having at least a signal at one two-theta values selected from 7.8±0.2, 9.7±0.2, 13.3±0.2, 15.7±0.2, 17.1±0.2, 18.9±0.2, 20.2±0.2 and 21.0±0.2.

In some embodiments, Triacid crystalline Form II is characterized by an X-ray powder diffractogram having at least a signal at three two-theta values selected from 7.8±0.2, 9.7±0.2, 13.3±0.2, 15.7±0.2, 17.1±0.2, 18.9±0.2, 20.2±0.2, and 21.0±0.2. In some embodiments, Triacid crystalline Form II is characterized by an X-ray powder diffractogram having at least a signal at two two-theta values selected from 7.8±0.2, 9.7±0.2, and 15.7±0.2. In some embodiments, Triacid crystalline Form II is characterized by an X-ray powder diffractogram having a signal at two-theta values of 7.8±0.2, 9.7±0.2, 13.3±0.2, 15.7±0.2, 17.1±0.2, and 18.9±0.2. In some embodiments, Triacid crystalline Form II is characterized by an X-ray powder diffractogram having a signal at two-theta values of 7.8±0.2, 9.7 0.2, 13.3±0.2, 15.7±0.2, 17.1±0.2, 18.9±0.2 and 20.2±0.2.

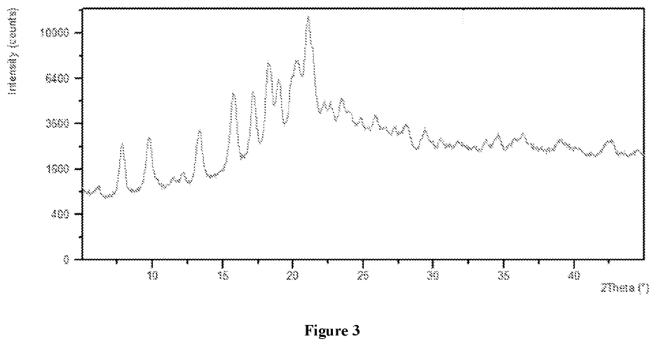

In some embodiments, Triacid crystalline Form II is characterized by an X-ray powder diffractogram substantially similar to that in FIG. 3.

### Step 3a: Alcohol-Z

Disclosed herein are processes for preparing Alcohol-Z (step 3a processes). The processes comprise reacting benzyl chloroformate (CbzCl) with aminoalcohol to form Alcohol-z:

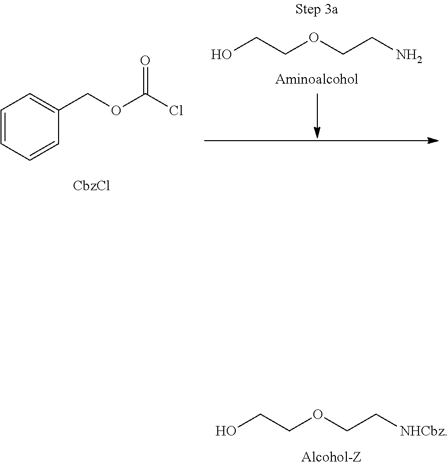 [SMILES: NCCOCCO ; "CbzCl" ; O=C(Cl)OCc1ccccc1 ; O=C(NCCOCCO)OCc1ccccc1]

The reaction occurs in the presence of a solvent and a base. Non-limiting solvents include polar aprotic solvents, aprotic solvents, and ethereal solvents. In some embodiments, the solvent is dichloromethane (DCM), dichloroethane, ethyl acetate, or methyl THF. In some embodiments, the solvents are polar aprotic solvents. In some embodiments, the solvent is DCM or dichloroethane. In some embodiments, the solvent is DCM. In some embodiments, the solvent is ethyl acetate or methyl THF. Non-limiting bases include tertiary amine bases, such as triethylamine (TEA), DIPEA, N-methylmorpholine, 1-methylimidazole and pyridine. In some embodiments, the base is a tertiary amine base. In some embodiments, the base is triethylamine (TEA), DIPEA, N-methylmorpholine, 1-methylimidazole and pyridine. In some embodiments, the base is TEA. In some embodiments, the solvent is DCM and the base is TEA. In some embodiments, the aminoalcohol is present at about 1.0-1.2 equivalents, relative to benzyl chloroformate. In some embodiments, the aminoalcohol is present at about 1.1 equivalents, relative to benzyl chloroformate. After the aminoalcohol solution (with solvent and base) is cooled to about −5 to 5° C., the CbzCl is added to the aminoalcohol solution over a period of time. In some embodiments, the CbzCl is added to the aminoalcohol over a period of time of about 50-70 minutes or 60 minutes, optionally at a temperature that maintained at about 0-12° C., 3-10° C., 8-11° C., 10° C., 5° C. or 0° C. Following the addition of CbzCl, the reaction temperature is adjusted to about 28-42° C. In some embodiments, the reaction temperature is adjusted to about 30-40° C., 33-37° C., or 35° C. In some embodiments, the reaction temperature is about 35° C. In some embodiments, the aminoalcohol is present at about 1.1 equivalents, relative to benzyl chloroformate and reaction temperature is about 20-30° C., 30-40° C., 33-37° C., or 35° C. In some embodiments, the aminoalcohol is present at about 1.1 equivalents, relative to benzyl chloroformate and reaction temperature is about 34-36° C. In some embodiments, the aminoalcohol is present at about 1.1 equivalents, relative to benzyl chloroformate and reaction is maintained at about 35° C.

In some embodiments, a step 3a process for preparing Alcohol-Z comprises reacting benzyl chloroformate (CbzCl) with aminoalcohol in presence of DCM as solvent and TEA as base. Following the addition of CbzCl over a time period of about 1 hour, the reaction temperature is adjusted from about 0-5 or 3-12° C. to about 35° C. In some embodiments, DCM is present at about 3-5 volumes. In some embodiments, the TEA is present at about 0.8-1.2 eq. The reaction continues for at least 12 hours, e.g., about 10-12 hours. In some embodiments, a step 3a process for preparing Alcohol-Z is shown in the below scheme:

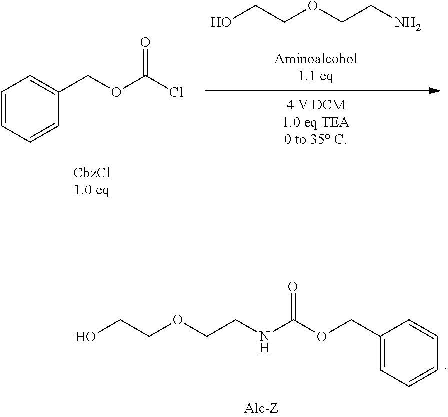 [SMILES: NCCOCCO ; O=C(Cl)OCc1ccccc1 ; O=C(NCCOCCO)OCc1ccccc1]

In some embodiments, the temperature is about 5° C. during addition of reagents and the reaction temperature is increased to about 35° C.

Following the reaction, Alcohol-Z product can be isolated by cooling to about room temperature (e.g., 20-25° C.), quenched (e.g., with water) and extracted with solvent (e.g., DCM), and optionally washed (e.g., 10% NaCl), and then followed by distillation. In some embodiments, the yield of Alcohol-Z is greater than or equal to about 95, 96, 97, 98, or 99% yield.

The Alcohol-Z Step 3a process described also reduces one or more impurities including benzyl alcohol, dibenzyl carbonate, benzyl chloroformate, and/or doubly protected Alcohol-Z, which are shown below.

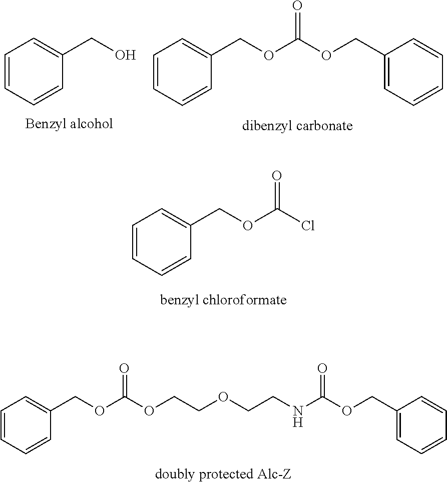

In some embodiments, step 3a produces Alcohol-Z with greater than about 90 or 95% purity as measured by liquid chromatography (LC). In some embodiments, the purity is greater than about 96, 97, 98, or 99%, as measured by LC. In some embodiments, impurities present at the end of reaction at an amount of less than or equal to about 10, 9, 8, 7, 6, 5, 4, 3, 2, or 1% as measured by LC.

### Step 3b: NAG-Z

Disclosed herein are processes of preparing NAG-Z (step 3b processes). The processes comprise a direct glycosylation of Alcohol-Z with Acyl GalNAc (also referred to as DGalNAc herein):

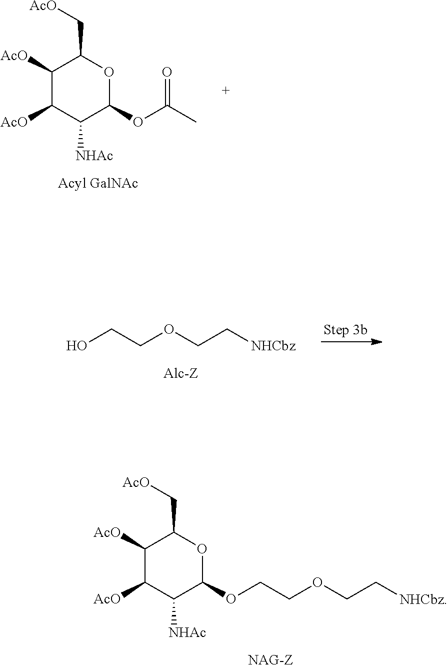 [SMILES: CC(=O)N[C@H]1[C@H](OC(C)=O)O[C@H](COC(C)=O)[C@H](OC(C)=O)[C@@H]1OC(C)=O ; O=C(NCCOCCO)OCc1ccccc1 ; CC(=O)N[C@H]1[C@H](OCCOCCNC(=O)OCc2ccccc2)O[C@H](COC(C)=O)[C@H](OC(C)=O)[C@@H]1OC(C)=O]

Without being bound by theory, an oxazoline intermediate is formed in situ and does not require steps to isolate the oxazoline intermediate, resulting in increased yield and decreased number of unit operations. The process of preparing NAG-Z (chemical name: (2R,3R,4R,5R,6R)-5-acetamido-2-(acetoxymethyl)-6-(2-(2-(((benzyloxy)carbonyl)amino)ethoxy)ethoxy)tetrahydro-2H-pyran-3,4-diyl diacetate) comprises reacting Acyl GalNAc (chemical name: (2S,3R,4R,5R,6R)-3-acetamido-6-(acetoxymethyl)tetrahydro-2H-pyran-2,4,5-triyl triacetate) with Alcohol-Z (chemical name: benzyl (2-(2-hydroxyethoxy)ethyl)carbamate) to produce NAG-Z.

Provided herein is a method for crystallizing NAG-Z comprising a solvent and antisolvent. In some embodiments, the solvent is acetonitrile and the antisolvent is water, methyl tert-butyl ether (MTBE), toluene, IPAc, or IPA. The process described also reduces one or more impurities including DGalOH and/or Ac-Z, which are shown below:

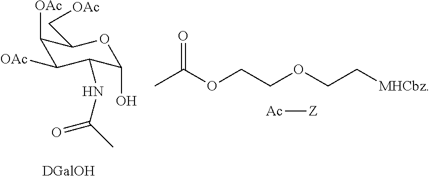

In some embodiments, the purity of NAG-Z is greater than about 96, 97, 98, or 99%, as measured by LC. In some embodiments, impurities present at the end of reaction at an amount of less than or equal to about 10, 9, 8, 7, 6, 5, 4, 3, 2, or 1% as measured by LC.

In some embodiments, the NAG-Z reaction is performed in the presence of an acid. In some embodiments, the acid is a Lewis acid. In some embodiments, the acid is TMSOTf. In some embodiments, the acid is not TMSOTf. Although TMSOTf is commonly used in glycosylation reactions, it was discovered that under certain conditions TMSOTf can degrade the NAG-Z product, resulting in lower yield ofNAG-Z. Non-limiting examples of acids include trifluoromethanesulfonic acid (also known as trifluoromethanesulfonate (CF₃SO₃) or triflate (Tf)), silyl triflates (e.g., TBSOTf, or TIPSOTf), metal triflates (e.g., bismuth triflate, indium triflate, copper triflate, scandium triflate, neodymium triflate, dysprosium triflate, ytterbium triflate, samarium triflate, lanthanum triflate, potassium triflate, magnesium triflate, or aluminum triflate), metal chlorides (e.g., FeCl₃, AlCl₃, InCl₃, or BiCl₃), trifluoroacetic acid (TFA), and BF₃OEt₂. In some embodiments, the acid is bismuth trifluoromethanesulfonate (Bi(OTf)₃), boron trifluoride etherate (BF₃OEt₂) tert-butyldimethylsilyl trifluoromethanesulfonate (TBSOTf), triisopropylsilyl trifluoromethanesulfonate (TIPSOTf), indium trifluoromethanesulfonate (In(OTf)₃), or copper trifluoromethanesulfonate (Cu(OTf)₂). In some embodiments, the acid is bismuth trifluoromethanesulfonate (Bi(OTf)₃), indium trifluoromethanesulfonate (In(OTf)₃), copper trifluoromethanesulfonate (Cu(OTf)₂), or tert-butyldimethylsilyl trifluoromethanesulfonate (TBSOTf). In some embodiments, the acid is bismuth trifluoromethanesulfonate (Bi(OTf₃), indium trifluoromethanesulfonate (In(OTf)₃), or copper trifluoromethanesulfonate (Cu(OTf)₂). In some embodiments, the acid is bismuth trifluoromethanesulfonate (Bi(OTf)₃) or copper trifluoromethanesulfonate (Cu(OTf)₂). In some embodiments, the acid is bismuth trifluoromethanesulfonate (Bi(OTf)₃). In some embodiments, the acid is copper trifluoromethanesulfonate (Cu(OTf)₂). In some embodiments, the acid is indium trifluoromethanesulfonate (In(OTf)₃). In some embodiments, the acid is indium trifluoromethanesulfonate (In(OTf)₃) or copper trifluoromethanesulfonate (Cu(OTf)₂). In some embodiments, the acid is tert-butyldimethylsilyl trifluoromethanesulfonate (TBSOTf). In some embodiments, the acid is present at about 0.01-0.1, 0.01-0.04, 0.05-01, 0.1-0.6, 0.1-0.4, 0.3-0.6, or 0.5-0.7 equivalents, relative to Alcohol-Z. In some embodiments, the acid is present at about 0.1-0.2 or 0.13-0.17 equivalents, relative to Alcohol-Z. In some embodiments, the acid is present at about 0.15 equivalents, relative to Alcohol-Z. In some embodiments, the acid is present at about 0.01-0.04 or 0.02-0.03 equivalents, relative to Alcohol-Z. In some embodiments, the acid is present at about 0.025 equivalents (or 2.5 mol %), relative to Alcohol-Z. In some embodiments, the acid is present at about 1-10, 1-4, or 2-3 mol %, relative to Alcohol-Z.

In some embodiments, the reaction is performed in the presence of a solvent. Non-limiting examples of solvents that can be used include acetonitrile, dichloroethane, dichloromethane, dimethylformamide (DMF), and DMSO. In some embodiments, the solvent is acetonitrile. In some embodiments, the solvent is dichloroethane or dichloromethane. Solvent volumes listed here are relative to Alcohol-Z. In some embodiments, the solvent is present at about 6-10 or 8-12 volumes in total. In some embodiments, the solvent is present at 8 volumes in total. In some embodiments, the solvent is present at 10 volumes in total. In some embodiments, the Acyl GalNAc is added to the reaction mixture as a Acyl GalNAc solution comprising Acyl GalNAc, acid, and solvent. In some embodiments, the reaction mixture already contains Alc-Z that was produced from the end of Step 3a. In some embodiments, the solvent in the Acyl GalNAc solution is about 2-4 or 4-6 volumes. In some embodiments, the solvent in the Acyl GalNAc solution is about 2-4 or 4-5 volumes or about 2, 3, 4, or 5 volumes. In some embodiments, the Alcohol-Z is added to the reaction as an Alcohol-Z solution comprising dried (e.g., dried azeotropically or with drying agents) Alcohol-Z and solvent. In some embodiments, the solvent in the Alcohol-Z solution is about 5 volumes.

In some embodiments, Acyl GalNAc is present at about 1.0-2.0 equivalents, 1.3-1.7 equivalents, or 1.4-1.6 equivalents, relative to Alcohol-Z. In some embodiments, the Acyl GalNAc is present at about 1.5 equivalents, relative to Alcohol-Z.

After Acyl GalNAc, Alcohol-Z, acid, and solvent are combined, the reaction temperature is heated to about 55-65, 65-75, or 70-90° C. In some embodiments, the reaction temperature is about 70-90° C. In some embodiments, the reaction temperature is about 75-85° C. In some embodiments, the reaction temperature is about 80° C. In some embodiments, the reaction temperature is about 58-62° C. In some embodiments, the reaction temperature is about 60° C. In some embodiments, the reaction time is about 10-20 hours. In some embodiments, the reaction time is about 12-18, 10-14 or 16-20 hours, or at least about 12, 14, 16, or 18 hours. Subsequently the reaction is cooled to about room temperature (e.g., about 20° C. or 15-25° C.) for filtration (e.g., polish filtration) and charcoal treatment. In some embodiments, the charcoal added is about 3-7 weight (wt) %, 5-10 wt %, 8-15 wt %, 13-25 wt %, 20-35 wt % 30-50 wt %, 40-60 wt %, or 50-70 wtt %. In some embodiments, the charcoal added is about 3-7 weight (wt) %, 5-10 wt %, or 8-15 wt %. In some embodiments, the charcoal added is about 30-50 wt %, 40-60 wt %, or 50-70 wt %. In some embodiments, the charcoal added is about 40 wt %, 50 wt %, or 60 wt %.

Following charcoal treatment of the NAG-Z solution, the charcoal is filtered and NAG-Z is crystallized by adding an antisolvent. In some embodiments, the antisolvent is MTBE, water, toluene, IPAc, or IPA. In some embodiments, the antisolvent is MTBE, water, or toluene. In some embodiments, the antisolvent is MTBE. In some embodiments, the antisolvent is added over a period of about 20-40 minutes or 30 minutes. In some embodiments, the antisolvent is added over a period of about 50-70 minutes or 60 minutes. A seed of NAG-Z is optionally added to the solution, which is optionally allowed to age overnight. In some embodiments, additional antisolvent is optionally added and crystallization proceeds for another a period of about 70-100 minutes or 90 minutes. The resulting wet cake is rinsed with additional solvent and antisolvent.

In some embodiments, a step 3b process for preparing NAG-Z is show in the below scheme:

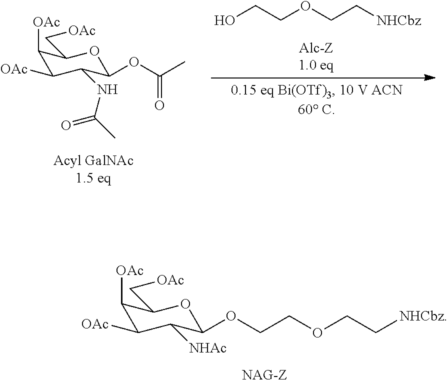 [SMILES: O=C(NCCOCCO)OCc1ccccc1 ; CC(=O)NC1C(OC(C)=O)OC(COC(C)=O)C(OC(C)=O)C1OC(C)=O ; CC(=O)NC1C(OCCOCCNC(=O)OCc2ccccc2)OC(COC(C)=O)C(OC(C)=O)C1OC(C)=O]

In some embodiments, a process of preparing NAG-Z is shown in the below scheme:

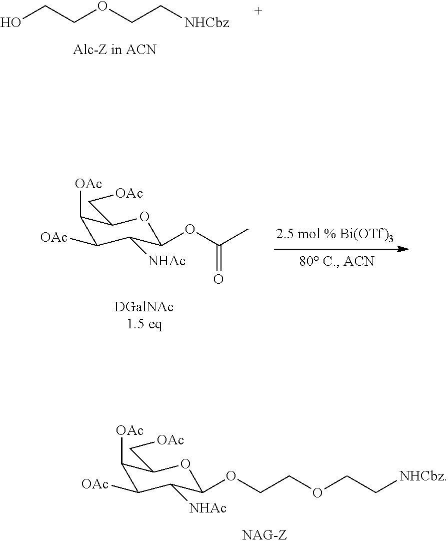 [SMILES: O=C(NCCOCCO)OCc1ccccc1 ; CC(=O)NC1C(OC(C)=O)OC(COC(C)=O)C(OC(C)=O)C1OC(C)=O ; CC(=O)NC1C(OCCOCCNC(=O)OCc2ccccc2)OC(COC(C)=O)C(OC(C)=O)C1OC(C)=O]

### Step 4a: NAG-H or Salt Thereof

Disclosed herein are processes to manufacture NAG-H, or salt thereof (step 4a processes). The processes comprise hydrogenation of NAG-Z in the presence of a Pd/C catalyst and a solvent (e.g., dimethylacetamide) to prepare NAG-H, or salt thereof:

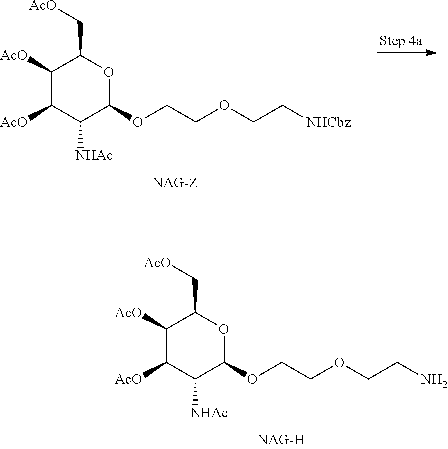 [SMILES: CC(=O)N[C@H]1[C@H](OCCOCCNC(=O)OCc2ccccc2)O[C@H](COC(C)=O)[C@H](OC(C)=O)[C@@H]1OC(C)=O ; CC(=O)N[C@H]1[C@H](OCCOCCN)O[C@H](COC(C)=O)[C@H](OC(C)=O)[C@@H]1OC(C)=O]

In some embodiments, NAG-H or salt thereof is optionally carried forward as a solution to the amide coupling reaction of Step 4b processes described herein. An advantage of this improved process avoids isolating NAG-H, or salt thereof, as a hygroscopic and difficult to handle solid. Additionally, the solvent volume used in this improved process has been reduced from a prior process.

In some embodiments, the NAG-H or salt thereof is NAG-H trifluoroacetic acid (TFA) salt, NAG-H oxalic acid salt, NAG-H tartaric acid salt, or NAG-H citric acid salt. In some embodiments, the NAG-H or salt thereof is NAG-H TFA (also called NAG-H TFA salt herein).

In some embodiments, a step 4a process comprises combining NAG-Z with Pd/C catalyst in solvent, adding acid, and pressurizing with hydrogen.

In some embodiments, Pd/C catalyst is 10, 9, 8, 7, 6, 5, 4, or 3% Palladium/Carbon. In some embodiments, the Pd/C catalyst is 5% Palladium/Carbon.

In some embodiments, the solvent is IPA, EtOH, DMAc, or DCM. In some embodiments, the solvent is DMAc. In some embodiments, the solvent is DCM. In some embodiments, solvent volume used is at about 3 volumes to about 6 volumes. In some embodiments, solvent volume used is at about 3 volumes to about 5 volumes. In some embodiments, DMAc is used at about 3 volumes to about 5 volumes. In some embodiments, DMAc is used at about 4 volumes. In some embodiments, DCM is used at about 3 volumes to about 6 volumes. In some embodiments, DCM is used at about 3-5 or 4-6 volumes.

In some embodiments, the acid is TFA, acetic acid (AcOH), Pivalic acid (PivOH). In some embodiments, the acid is TFA. In some embodiments, the acid is present at about 0.8-1.2 eq, relative to NAG-Z. In some embodiments, TFA is present at about 0.8-1.2 eq or 1.0 eq, relative to NAG-Z.

In some embodiments, the reaction is pressurized with hydrogen at about 30-60 psig or 40-50 psig. In some embodiments, the reaction is pressurized with hydrogen at about 35, 40, 45, 50, or 55 psig.

In some embodiments, the reaction is pressurized with hydrogen at about 45 psig.

In some embodiments, step 4a is performed at a temperature of about 20° C. to about 25° C. In some embodiments, the temperature is about 20° C. to about 24° C. In some embodiments, the temperature is about 20° C. to about 23° C. In some embodiments, the temperature is about 20° C. to about 22° C.

In some embodiments, reaction occurs for about 15-25 hours or 18-20 hours.

### Step 4b: TGZ

Described herein are processes of preparing TGZ (step 4b processes). TGZ is synthesized by coupling three NAG-H units to the Triacid via concurrent amide couplings:

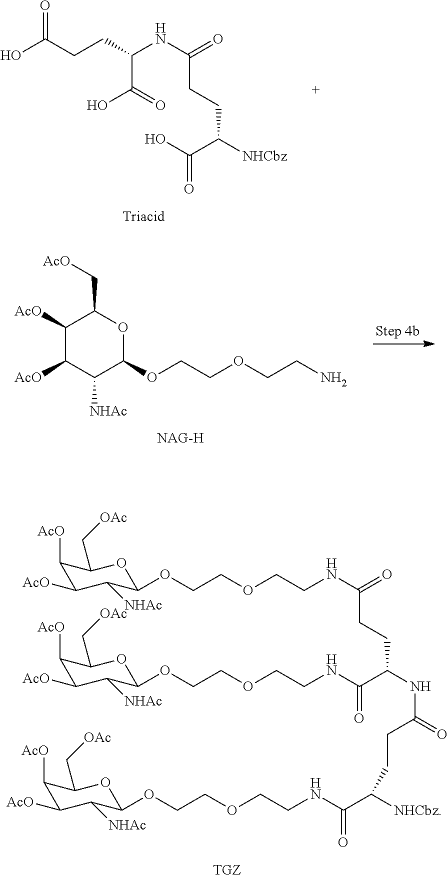 [SMILES: O=C(O)CC[C@H](NC(=O)CC[C@H](NC(=O)OCc1ccccc1)C(=O)O)C(=O)O ; CC(=O)N[C@H]1[C@H](OCCOCCN)O[C@H](COC(C)=O)[C@H](OC(C)=O)[C@@H]1OC(C)=O ; CC(=O)NC1C(OCCOCCNC(=O)CC[C@H](NC(=O)CC[C@H](NC(=O)OCc2ccccc2)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]

In a prior process for Step 4b, tri-GalNAc-Cbz (TGZ) had been isolated and purified via a solvent-intensive and low-yielding chromatography step. Disclosed herein are improved step 4b processes that comprises chromatography-free isolation and purification of the TGZ intermediate by precipitation from solvent. An advantage of the improved step 4b processes is elimination of chromatography resulting in increased TGZ yield, e.g., greater than about 85%, and saving time and money, e.g., by reducing solvent usage and/or avoiding specialized manufacturing equipment.

The improved TGZ purification process additionally provides more efficient amide coupling. The improved step 4b process comprises NAG-H coupling to the Triacid using a coupling reagent and in a given solvent (e.g., TBTU in dimethylacetamide) which allowed the equivalents of NAG-H used in the reaction to be lowered (e.g., from about 3.5 to about 3-3.3 or 3.2). Improved step 4b also includes mild buffer washes that minimizes formation of impurity side products (e.g., des-acyl side product). Improved step 4b uses a mild buffer (e.g., sodium or potassium phosphate buffer) instead of harsh acid/base aqueous washes. Finally, a single precipitation of TGZ from solvent system (e.g., DCM/MTBE) was implemented, providing a more efficient and higher yielding (e.g., greater than about 80-90% yield) isolation of TGZ as an amorphous solid with higher purity (e.g., greater than about 95% purity as measured by LC).

In some embodiments, a step 4b process comprises: combining Triacid with NAG-H or salt thereof, which can optionally be in a solvent, adding a base, and adding a coupling reagent. In some embodiments of step 4b, the process further comprises extraction/work-up, distillation, crystallization, precipitation, filtration, and/or drying. In some embodiments, the NAG-H or salt there of is NAG-H TFA.

In some embodiments, the coupling reagent is 2-(1H-Benzotriazole-1-yl)-1,1,3,3-tetramethylaminium tetrafluoroborate (TBTU), N-[(Dimethylamino)-1H-1,2,3-triazolo-[4,5-b]pyridin-1-ylmethylene]-N-methylmethanaminium hexafluorophosphate N-oxide (HATU), 0-(7-Azabenzotriazole-1-yl)-N,N,N′,N′-tetramethyluronium tetrafluoroborate (TATU), 0-(Benzotriazol-1-yl)-N,N,N′,N′-tetramethyluronium hexafluorophosphate (HBTU), Chloro-N,N,N′,N′-tetramethylformamidinium hexafluorophosphate (TCFH), (1-Cyano-2-ethoxy-2-oxoethylidenaminooxy)dimethylamino-morpholino-carbenium hexafluorophosphate (COMU), (3-Dimethylamino-propyl)-ethyl-carbodiimide (EDC), Ethyl cyano(hydroxvimino)acetate (Oxyma), 1-Hydroxybenzotriazole (HOBt), [Ethyl cyano(hydroxyimino)acetato-02]tri-1-pyrrolidinylphosphonium hexafluorophosphate (PyOxim), Propylphosphonic anhydride (T3P), Phosphoric acid bis(2-oxooxazolidide) chloride (BOPCl), 2-Chloro-4,6-dimethoxy-1,3,5-triazine (CDMT), Dicyclohexylcarbodiimide (DCC), Diisopropylcarbodiimide (DIC), 7-Azabenzotriazol-1-yloxy)tripyrrolidinophosphonium hexafluorophosphate (PyAOP), 0-[(Ethoxycarbonyl)cyanomethyleneamino]-N,N,N′N′-tetramethyluronium tetrafluoroborate (TOTU), and N,N,N′,N′-Tetramethyl-O—(N-succinimidyl)uronium tetrafluoroborate (TSTU). In some embodiments, the coupling reagent is TBTU or HATU. In some embodiments, the coupling reagent is TCFH. In some embodiments, the coupling reagent is HATU. In some embodiments, the coupling reagent is TBTU.

In some embodiments, the base is Triethylamine, Diisopropylethylamine (DIPEA), N-methylimidazole (NMI), N-methylmorpholine (NMM), or 2,2,6,6-tetramthylpiperidine (TMP). In some embodiments, the base is DIPEA, NMI, NMM. In some embodiments, the base is NMI.

In some embodiments, the coupling reagent is TBTU and the base is NMI. In some embodiments, the coupling reagent is TCFH and the base is NMI.

In some embodiments, the solvent is Dichloromethane (DCM), Dimethylformamide (DMF), Acetonitrile (MeCN), Dimethylacetamide (DMAc), or combination thereof. In some embodiments, the solvent is DMF or DMAc. In some embodiments, the solvent is DCM or DMAc. In some embodiments, the solvent is DCM. In some embodiments, the solvent is DMF. In some embodiments, the solvent is DMAc.

In some embodiments, the coupling reagent is TBTU and solvent is DMF. In some embodiments, the coupling reagent is TBTU and solvent is DMAc. In some embodiments, the coupling reagent is TBTU and solvent is DCM.

In some embodiments, the NAG-H or salt thereof is about 3-4 equivalents. In some embodiments, the NAG-H or salt thereof is about 3-3.7 equivalents. In some embodiments, the NAG-H or salt thereof is about 3.1-3.3 equivalents. In some embodiments, the NAG-H or salt thereof is NAG-H TFA. The foregoing equivalents are relative to Triacid.

In some embodiments, the base equivalence is about 6-15 eq, 8-12 eq, or 10-12 eq. In some embodiments, the base equivalence is about 10-12 eq. In some embodiments, the base equivalence is about 11.5-12.5 eq. In some embodiments, the base equivalence is about 11-12 eq. In some embodiments, the base equivalence is about 12 eq. In some embodiments of the foregoing embodiments of base, the base is NMI. In some embodiments of the foregoing embodiments of base, the base is 12 equivalents of NMI. The foregoing equivalents are relative to Triacid.

In some embodiments, a step 4b process for preparing TGZ comprises reacting the Triacid with 3.2 equivalents of NAG-H or salt thereof, 3.5 equivalents of TBTU, and 12.0 equivalents of NMI. The foregoing equivalents are relative to Triacid.

In some embodiments, a step 4b process for preparing the TGZ is shown in the below scheme:

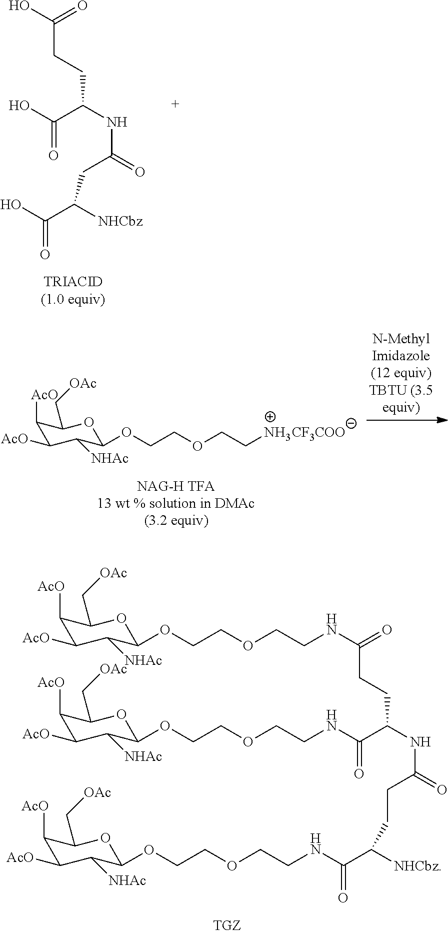

In some embodiments, the step 4b processes described herein produces high purity of TGZ with a low percentage of des-acyl impurity. In some embodiments, the TGZ has a purity of greater than about 93, 94, 95%. In some embodiments, the resulting TGZ comprises less than about 1, 0.5, or 0.2% of des-acyl impurity. In some embodiments, the resulting TGZ comprises less than about 1% of des-acyl impurity.

In some embodiments, the step 4b process further comprises at least one, two, or three washes with acid followed by at least one, two, or three washes with base. In some embodiments, the acid is HCl and the base is NH₄OH. In some embodiments, HCl washes and NH₄OH washes are used when the coupling agent is TCFH, base is NMI, and solvent is DCM.

In some embodiments, the step 4b process further comprises at least one, two, or three washes with a buffer. In some embodiments, the buffer is a phosphate buffer. In some embodiments, the buffer has a pH of about 4 to 8. In some embodiments, the buffer has a pH of about 5-7. In some embodiments, the buffer has a pH of about 6. In some embodiments, the pH 6 phosphate buffer wash is performed at least 2 or 3 times. In some embodiments, the pH 6 phosphate buffer wash is performed 3 times. In some embodiments, the buffer is a sodium phosphate or potassium phosphate. In some embodiments, the buffer is potassium phosphate. In some embodiments, the buffer is about 20 to 550 mM potassium phosphate. In some embodiments, the buffer is about 25 to 500 mM potassium phosphate. In some embodiments, the buffer is about 40 to 60 mM potassium phosphate. In some embodiments, the buffer is about 50 mM potassium phosphate. In some embodiments, the buffer is about 50 mM potassium phosphate at about pH 5-7. In some embodiments, the buffer is about 50 mM potassium phosphate at about pH 6.

In some embodiments, step 4b process comprises azeotropic distillation of the organic phase with a solvent. In some embodiments, the solvent for distillation is DCM.

In some embodiments, the step 4b process further comprises precipitation of TGZ from the solvent using an anti-solvent. In some embodiments, the solvent is DCM and the anti-solvent is an ethereal solvent. In some embodiments, the ethereal solvent is Dimethoxyethane (DME), 2-Methyltetrahydrofuran (MeTHF or 2-MeTHF), or Methyl tert-butyl ether (MTBE). In some embodiments, the solvent is DCM and the anti-solvent is MTBE. In some embodiments, the solvent is DCM and the anti-solvent is isopropyl acetate (IPAc) or ethyl acetate (EtOAc). In some embodiments, the solvent is DCM and the anti-solvent is DME. In some embodiments, the solvent is DMSO or dimethylacetamide (DMAc) and the anti-solvent is 2-MeTHF. In some embodiments, the solvent is DMF and the anti-solvent is Isopropyl acetate (IPAc). In some embodiments, TGZ is precipitated using a final solvent ratio of about 1:1 ratio of solvent:antisolvent. In some embodiments, TGZ is precipitated using a final solvent ratio about 1:1 ratio of DCM:MTBE. In some embodiments, antisolvent (e.g., MTBE) addition occurred over about 1.5-2.5, 2-3, 2.5-3.5, or 3-4 hours or about 2, 3, or 4 hours.

In some embodiments, TGZ is obtained in at least about 88%, 89%, 90% yield. In some embodiments, TGZ is obtained in at least about 95% purity as measured by LC (95 LCAP). In some embodiments, TGZ is obtained in at least about 96%, 97%, 98% purity as measured by LC.

In some embodiments, a step 4b process for preparing the TGZ comprises triacid (1.0 eq), NAG-H TFA (3.5 eq), DMAc, NMI (12 eq), TBTU (4.0 eq). In some embodiments, the step 4b process further comprises 50 mM pH 6 phosphate buffer washes and DCM washes. In some embodiments, step 4b further comprises precipitating TGZ upon addition of MTBE to the solution comprising DCM.

The improved step 4b processes result in reduced, minimal or undetected amounts of side products of the process. The side products of the process include mono-NAG, DI-NAG, DI-NAG-AZLACTONE, Des-Acyl (also referred to as des-acetate), Di-NAG-OH, Di-NAG-OAc, NAG-H guanidine, and NAG-H trifluoroacetamide, each of which are shown below.

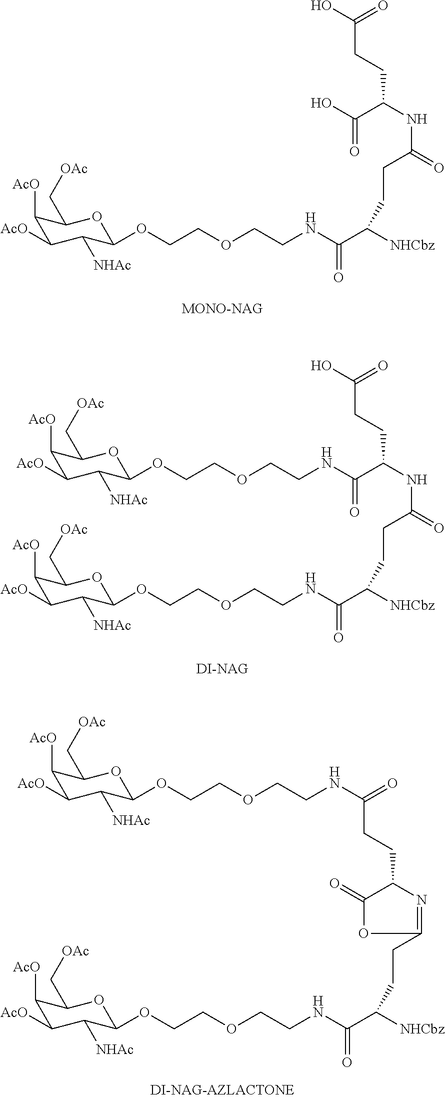 [SMILES: CC(=O)NC1C(OCCOCCNC(=O)[C@H](CCC(=O)N[C@@H](CCC(=O)O)C(=O)O)NC(=O)OCc2ccccc2)OC(COC(C)=O)C(OC(C)=O)C1OC(C)=O ; CC(=O)NC1C(OCCOCCNC(=O)[C@H](CCC(=O)O)NC(=O)CC[C@H](NC(=O)OCc2ccccc2)C(=O)NCCOCCOC2OC(COC(C)=O)C(OC(C)=O)C(OC(C)=O)C2NC(C)=O)OC(COC(C)=O)C(OC(C)=O)C1OC(C)=O ; CC(=O)NC1C(OCCOCCNC(=O)CC[C@@H]2N=C(CC[C@H](NC(=O)OCc3ccccc3)C(=O)NCCOCCOC3OC(COC(C)=O)C(OC(C)=O)C(OC(C)=O)C3NC(C)=O)OC2=O)OC(COC(C)=O)C(OC(C)=O)C1OC(C)=O]

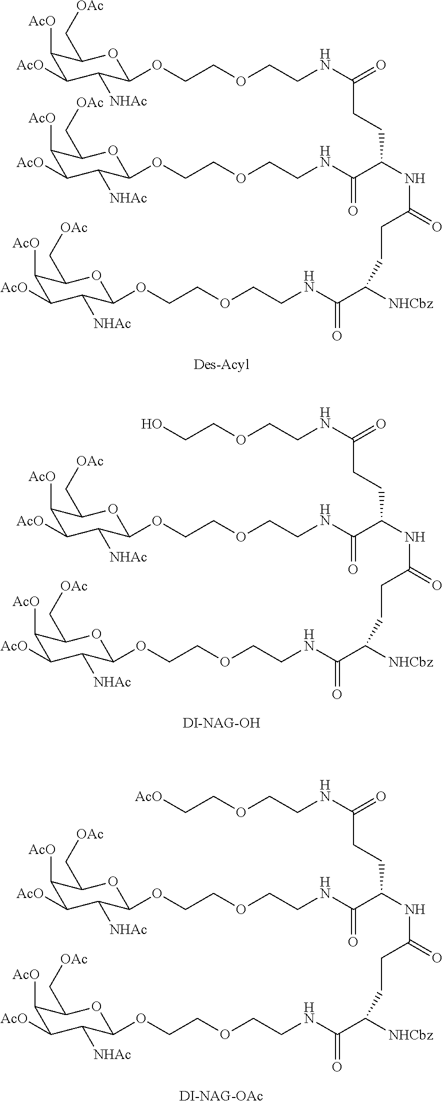

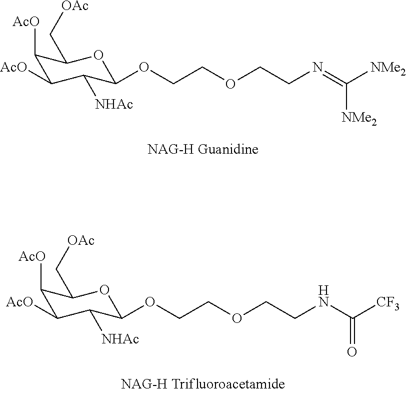 [SMILES: CC(=O)NC1C(OCCOCCN=C(N(C)C)N(C)C)OC(COC(C)=O)C(OC(C)=O)C1OC(C)=O ; CC(=O)NC1C(OCCOCCNC(=O)C(F)(F)F)OC(COC(C)=O)C(OC(C)=O)C1OC(C)=O]

In some embodiments, the step 4b processes results in TGZ with less than about 5%, 3%, 2%, 1, or 0.5% as measured by LC of Mono-NAG, Di-NAG, and/or Di-NAG-azlactone. In some embodiments, the step 4b process results in TGZ with less than about 2% of NAG-H guanidine, less than about 2% of Di-NAG-OH, less than about 1% of Di-NAG, about less than 1% Des-acyl and less than about 1% of of Di-NAG-OAc. Disclosed are compositions comprising TGZ with any of the impurities described that is less than or equal to 5% (e.g., 5, 4, 3, 2, 1%) as measured by LC.

In some embodiments, the processes results in a final TGZ yield of greater than about 85, 86, 87, 88, 89, or 90% yield. In some embodiments, the processes results in a final TGZ yield of greater than about 90% yield.

### Alternative Processes to TGZ: Step A-1, Step A-2, Step A-3

Step A-1: Step A-1 produces a Methyl Core (chemical name: dimethyl ((S)-4-(((benzyloxy)carbonyl)amino)-5-methoxy-5-oxopentanoyl)-L-glutamate) shown below and comprises an amide coupling between Z-L-GluOMe (chemical name: (S)-4-(((benzyloxy)carbonyl)amino)-5-methoxy-5-oxopentanoic acid) and di(OMe)Glu (chemical name: dimethyl L-glutamate hydrochloride):

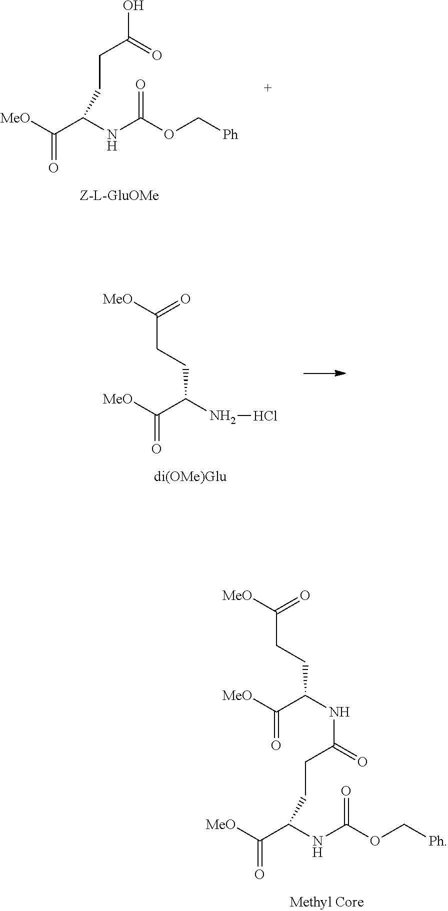 [SMILES: COC(=O)[C@H](CCC(=O)O)NC(=O)OCc1ccccc1 ; COC(=O)CC[C@H](NCl)C(=O)OC ; COC(=O)CC[C@H](NC(=O)CC[C@H](NC(=O)OCc1ccccc1)C(=O)OC)C(=O)OC]

In some embodiments, the reaction is performed in the presence of a base and coupling reagent. In some embodiments, the base is added to a z-L-GluOMe solution prior to addition of the coupling reagent and di(OMe)Glu to the reaction. In some embodiments, the base is N-Methylmorpholine (NMM) or di-isopropylethylamine. In some embodiments, the base is NMM.

In some embodiments, the reaction further comprises a solvent. In some embodiments, the solvent is an ethereal solvent. In some embodiments, the solvent is THF or MeTHF. In some embodiments, the solvent is THF. In some embodiments, the solvent is added to the reaction under positive pressure of nitrogen.

In some embodiments, the coupling reagent is isobutyl chloroformate (IBCF), TBTU, HATU, EDC, or DCC. In some embodiments, the coupling reagent is IBCF.

In some embodiments, the reaction temperature prior to and during the reaction with the coupling reagent is about −20° C. to about −10° C. In some embodiments, that reaction temperature is about −18° C. to about −13° C. In some embodiments, that reaction temperature is about −15° C.

In some embodiments, after that reaction temperature is reached, coupling reagent is added to the solution of z-L-Glu-OMe and base prior to addition of di(OMe)Glu to the reaction.

In some embodiments, the reaction time after di(OMe)Glu is about 50-70 minutes. In some embodiments, the reaction time is about 60 minutes.

In some embodiments, the di(OMe)Glu is added to the reation. In some embodiments, the amount of di(OMe)Glu is added in 4-6 equal portions. In some embodiments, the amount of di(OMe)Glu is added in 5 equal portions.

In some embodiments, the reaction temperature after addition of di(OMe)Glu is increased to about 5° C. to about 15° C. In some embodiments, the reaction temperature is increased to about 7° C. to about 12° C. In some embodiments, the reaction temperature is increased over a period of 2 hours.

In some embodiments, the reaction time is about 12-24 hours. In some embodiments, the reaction time is about 14-18 hours.

In some embodiments, the reaction further comprises an additional charge of coupling reagent, base, and/or z-L-Glu-OMe. In some embodiments, the reaction further comprises an additional charge of IBCF, NMM and Z-L-Glu-OMe. In some embodiments, the additional charges are about 8-12 mol %. In some embodiments, the additional charges are about 10 mol %.

In some embodiments, relative to z-L-GluOMe, the equivalents (eq.) of di(OMe)Glu is about 0.8 eq to about 1.3 eq, NMM, is about 2.3 eq to about 2.7 eq, and IBCF is about 0.8 eq to about 1.3 eq. In some embodiments, relative to z-L-GluOMe, the equivalents of di(OMe)Glu is about 1 eq., NMM is about 2.5 eq, and IBCF is about 1 eq.

After completion, the reaction is diluted with an organic solvent then washed sequentially with aqueous acid then aqueous base. In some embodiments, the organic solvent is an ester acetate solvent. In some embodiments, the organic solvent is ethyl acetate (EtOAc). In some embodiments, the aqueous acid is HCl. In some embodiments, the aqueous base is NaOH. In some embodiments, the aqueous washes are performed about 2 to about 4 times. Residual water is then removed from the organic solution before crystallization.

Anti-solvent addition crystallization is performed with a slow cooling ramp to room temperature. In some embodiments, the anti-solvent is heptane. In some embodiments, the solvent/anti-solvent is EtOAc/Heptane. In some embodiments, the solvent/anti-solvent is 1:1, 1:2, or 1:3 EtOAc/Heptane. In some embodiments, the solvent/anti-solvent is 1:1 EtOAc/Heptane The resulting slurry was filtered to isolate the Methyl Core solids. The solids were washed with organic solvent then dried under Nitrogen.

In some embodiments, a step A-1 process for preparing the methyl core is shown in the below scheme:

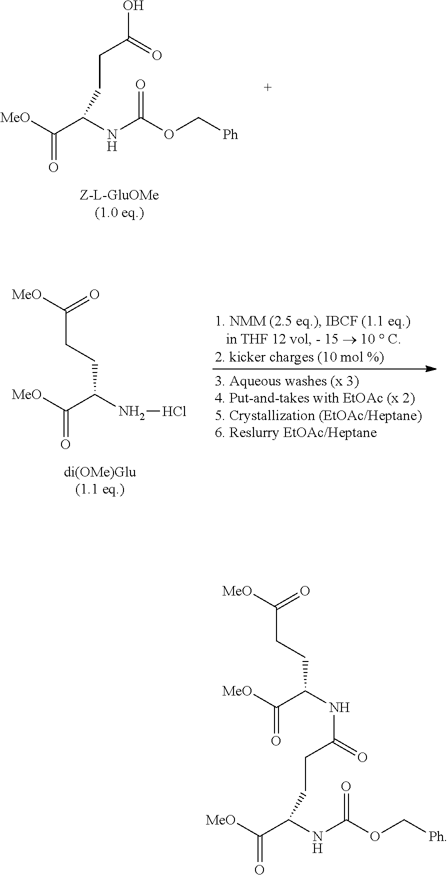 [SMILES: COC(=O)[C@H](CCC(=O)O)NC(=O)OCc1ccccc1 ; COC(=O)CC[C@H](NCl)C(=O)OC ; COC(=O)CC[C@H](NC(=O)CC[C@H](NC(=O)OCc1ccccc1)C(=O)OC)C(=O)OC]

Step A-2: Disclosed herein is a Triol compound (chemical name: benzyl ((10S,15S)-1,22-dihydroxy-10-((2-(2-hydroxyethoxy)ethyl)carbamoyl)-7,12,16-trioxo-3,20-dioxa-6,11,17-triazadocosan-15-yl)carbamate) and a process for preparing the Triol. Also disclosed herein is the unprotected Triol that does not have a CBz protecting group on the —NH₂. Step A-2 produces the Triol shown below and comprises aminolysis between the Methyl Core (dimethyl ((S)-4-(((benzyloxy)carbonyl)amino)-5-methoxy-5-oxopentanoyl)-L-glutamate) and an amino alcohol (chemical name: 2-(2-aminoethoxy)ethanol or 2-(2-aminoethoxy)ethan-1-ol):

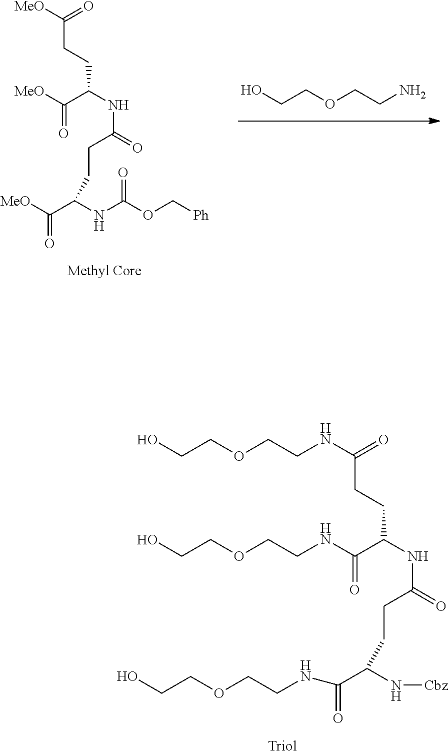

In some embodiments, the 2-(2-aminoethoxy)ethanol is neat and the reaction does not use a solvent. In some embodiments, solvent additives are used.

In some embodiments, relative to the Methyl Core, the 2-(2-aminoethoxy)ethanol is about 25-30 equivalents.

In some embodiments, the reaction temperature is about 25° C. to about 35° C. In some embodiments, the reaction temperature is about 30° C. In some embodiments, the reaction time is about 22-26 hours. In some embodiments, the reaction time is about 24 hours.

In some embodiments, Step A-2 is not enzyme-mediated. In some embodiments, Step A-2 is enzyme-mediated. In some embodiments, the enzyme is Novozym 5103 (liquid), Lipozyme CALB L (liquid), Resinase HT (liquid), Lipozyme TL (liquid), Novozym 435 (solid supp.), Novozym 40086 (solid supp.), Lipozyme TL (solid supp.), Papain (lyophilized), Trypsin (lyophilized), Amano PS (powder), Novozym 5103 (liquid), Lipozyme CALB L (liquid), Reinase HT (liquid), Aldolase (lyophilized), Papain (lyophilized), Trypsin (lyophilized), or Palatase (liquid). In some embodiments, the enzyme is Lipozyme CALB L (liquid), Lipozyme TL (liquid), Novozym 435 (solid supp.), Lipozyme TL (solid supp.). In some embodiments, the enzyme is Lipozyme CALB L (liquid). In some embodiments, the enzyme is Lipozyme TL (liquid). In some embodiments, the enzyme is Novozym 435 (solid supp.). In some embodiments, the enzyme is Lipozyme TL (solid supp.).

In some embodiments, an anti-solvent is added to precipitate the Triol. In some embodiments, the anti-solvent is ethyl acetate, methyl-tert-butyl ether, or isopropylacetate. In some embodiments, the anti-solvent is ethyl acetate.

In some embodiments, Triol seed crystal is optionally added to the reaction. In some embodiments, about 1-3 weight percent (wt %) of seed crystal is added. In some embodiments, about 2 weight percent (wt %) of seed crystal is added.

The reaction temperature is decreased to about room temperature. In some embodiments, the reaction temperature is decreased to about 20° C. to about 25° C. The slurry of Triol is allowed to age for about 16-20 hours or about 18 hours.

The slurry is filtered then the crude product solids is washed with organic solvent then redissolved in organic solvent for a re-slurry at temperature. The slurry is filtered and the product solids is washed with organic solvent then dried under nitrogen. In some embodiments, the organic solvent is ethyl acetate.

In some embodiments, a step A-2 process for preparing the Triol is shown in the below scheme:

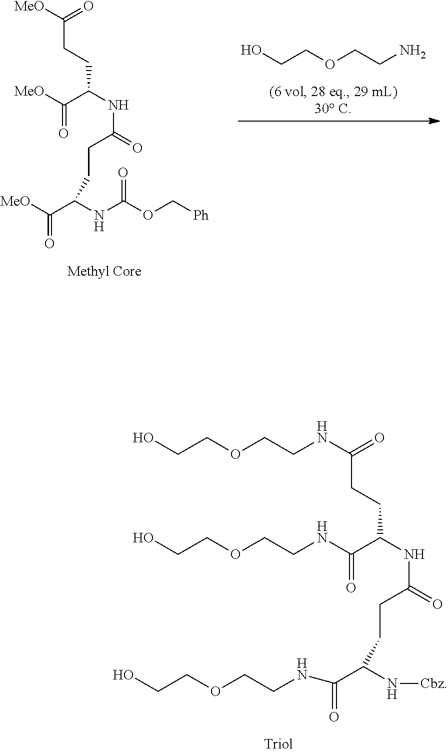

Step A-3: Step A-3 produces TGZ and comprises glycosylation between the Triol and B-D-galactosamine pentaacetate:

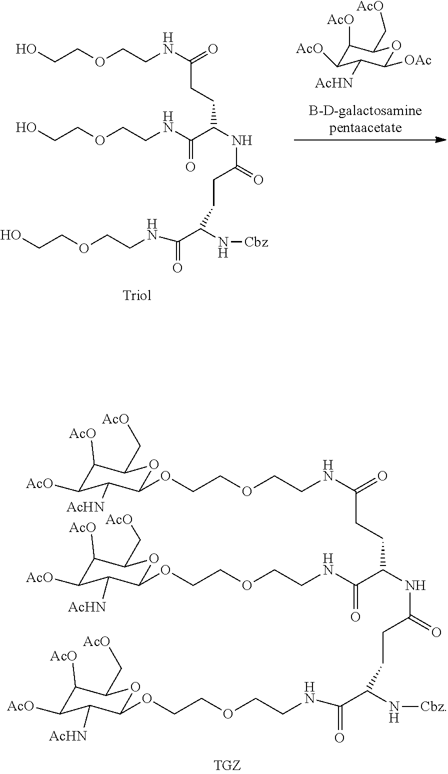

B-D-galactosamine pentaacetate is converted into an oxazoline solution (i.e., oxazoline derivative of B-D-galactosamine pentaacetate) shown below.

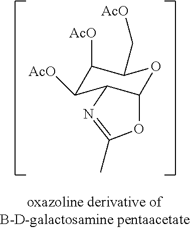

In some embodiments, the oxazoline derivative of B-D-galactosamine pentaacetate is prepared in the same reaction vessel as the Triol, instead of being separately prepared.

In some embodiments, B-D-galactosamine pentaacetate is first converted into an oxazoline solution (i.e., oxazoline derivative of B-D-galactosamine pentaacetate) prior to adding to a solution of Triol.

The oxazoline solution is produced by reacting B-D-galactosamine pentaacetate with Trimethylsilyl trifluoromethanesulfonate (TMSOTf). In some embodiments, TMSOTf is about 1 to about 1.5 eq. In some embodiments, TMSOTf is used in about 1.2 eq. The equivalents are relative to B-D-galactosamine pentaacetate. In some embodiments, the reaction temperature is about 35° C. to about 45° C.

In some embodiments the reaction temperature is about 40° C. In some embodiments, the reaction time is about 70 to about 110 minutes. In some embodiments, the reaction time is about 90 minutes.

In some embodiments, the reaction to prepare the oxazoline solution further comprises a solvent. In some embodiments, the solvent is dichloroethane (DCE) or dichloromethane (DCM). In some embodiments, the solvent is DCE.

In some embodiments, reaction temperature of the oxazoline solution is decreased to about 20° C. to about 28° C. In some embodiments, such reaction temperature is about 23° C.

In some embodiments, the Triol is mixed with sodium bicarbonate (NaHCO₃) to produce a slurry mixture of Triol and NaHCO₃ prior to addition of Beta-D-Galactosamine pentaacetate or its oxazoline solution. In some embodiments, the slurry mixture of Triol and NaHCO₃ further comprises a solvent. In some embodiments, the solvent for the slurry mixture is dichloromethane (DCM), dichloroethane, or acetonitrile. In some embodiments, the solvent for the slurry mixture is dichloromethane (DCM).

In some embodiments, the oxazoline solution is added to the slurry mixture of triol and NaHCO3. In some embodiments, the oxazoline solution is added to the mixture of triol and NaHCO3 at a rate of about 0.8 to about 1.2 equivalents of the oxazoline solution/hour up to about 4-5 equivalents/hour.

In some embodiments, the oxazoline solution is added to the mixture oftriol and and NaHCO₃ at a rate of about 1.0 equivalent of the oxazoline solution/hour up to about 4.5 equivalents/hour.

Afterwards, the reaction proceeds for about 18 to about 30 hours or overnight. In some embodiments, the reaction proceeds for about 24 hours. In some embodiments, the reaction temperature is about 20° C. to about 30° C. In some embodiments, the reaction temperature is about 25° C.

Additional charges of re-quantified oxazoline solution can be added to help with reaction conversion. The reaction is quenched with an organic base, followed by an aqueous base. In some embodiments, the organic base is Et₃N. In some embodiments, the aqueous base is NaHCO₃.

The organic phase is washed with a mild acid aqueous wash. In some embodiments, the acid is NH₄Cl. The organic solution was dried to remove residual water.

The resulting TGZ product is precipitated from a solvent/antisolvent system. In some embodiments, the solvent is DCM and the anti-solvent is dimethoxyethane (DME). The TGZ slurry was filtered, the product solids is washed with organic solvent and dried under Nitrogen. In some embodiments, the organic solvent is DCM/DME 1:1. To reach higher purity the solids can be reslurried in organic solvent at temperature, filtered, washed with organic solvent then dried under nitrogen.

In some embodiments, a step A-3 process for preparing the TGZ is shown in the below scheme:

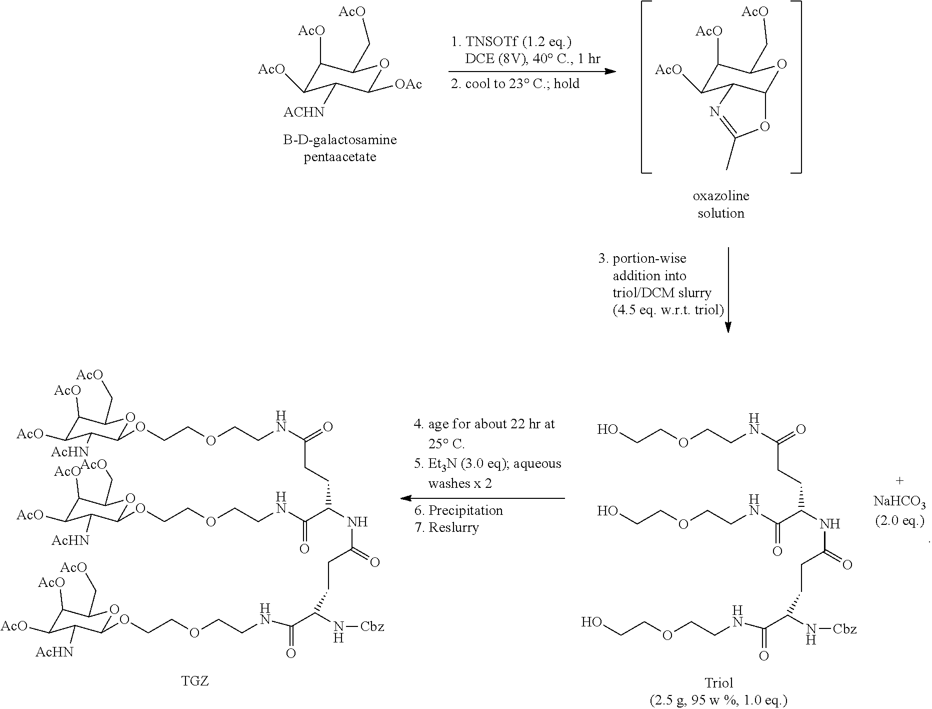

### Step 5a: TG Amine or Salt Thereof

Disclosed herein are processes for preparing TG Amine or a salt thereof (Step 5a processes). The processes comprise high pressure hydrogenolysis of TGZ, resulting in TG Amine or a salt thereof:

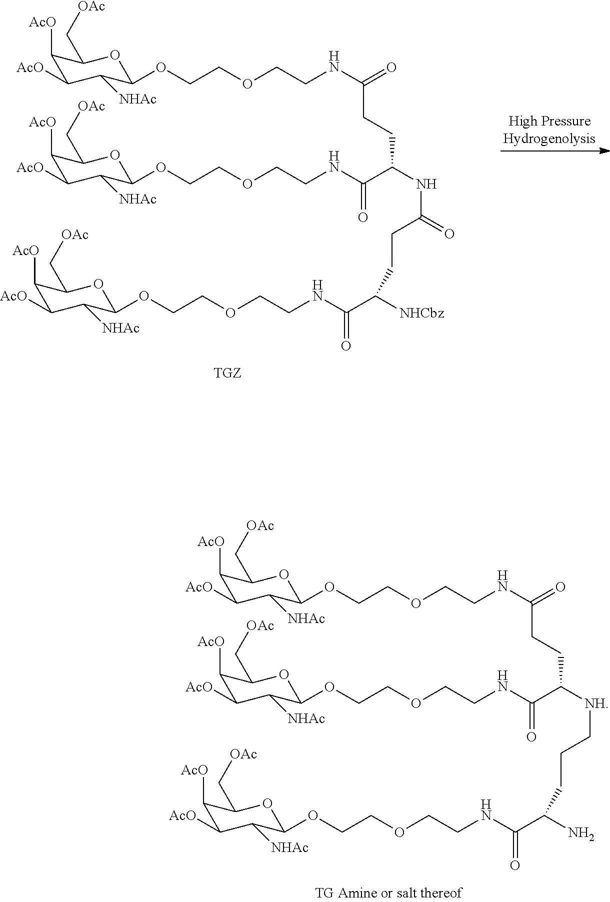 [SMILES: CC(=O)NC1C(OCCOCCNC(=O)CC[C@H](NC(=O)CC[C@H](NC(=O)OCc2ccccc2)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O ; CC(=O)NC1C(OCCOCCNC(=O)CC[C@H](NCCC[C@H](N)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]

In a prior step 5a process, the hydrogenolysis of TGZ to TG Amine, or salt thereof, had some undesirable factors such as high catalyst loading (e.g., 10%) of an expensive Pd-source for catalyst or flammable reaction solvent MeOH (when exposed to hydrogen).

Disclosed herein is are improved processes comprising a high pressure hydrogenolysis for a carboxybenzyl deprotection of TGZ to form a TG Amine or a salt thereof. The improvements include use of DCM as a reaction solvent to eliminate an isolation and reaction kinetic analysis to determine optimal catalyst loading and stir speed. The TG Amine or salt thereof can be telescoped (without isolation) into the next Step 5b processes described herein.

In some embodiments, a step 5a process comprises: combining TGZ with Pd/C catalyst and pressurizing with hydrogen. In some embodiments, the process further comprises acid. In some embodiments, the process comprises the step of combining TGZ with solvent, adding Pd/C catalyst, adding acid, and pressurizing with hydrogen. In some embodiments, a step 5a process comprises: combining TGZ with Pd/C catalyst, adding acid and solvent, and pressurizing with hydrogen. Following reaction completion, the catalyst can be removed through filtration. In some embodiments of step 5a, the process further comprises filtration.

A palladium source is used for hydrogenolysis. In some embodiments, the palladium source is a Pd/C catalyst. In some embodiments, the palladium on carbon (Pd/C) catalyst used is less than about 10%, 9%, 8%, 7%, 6% Pd/C. In some embodiments, the Pd/C catalyst is about 4-6% Pd/C. In some embodiments, the Pd/C catalyst is 5% Pd/C.

In some embodiments, the process comprises the use of an acid. In some embodiments, the acid is TFA, oxalic acid, HCl, AcOH, H₃PO₄, citric. In some embodiments, the acid is TFA or oxalic acid. In some embodiments, the acid is TFA. In some embodiments, the acid is oxalic acid. In some embodiments, the acid is added at am amount of about 0.8-1.1 or 1.0, eq, or at an amount that is not greater than about 1.0 or 1.1 eq. Over addition of acid in step 5a processes generally results in higher levels of the TGAmine-TFA acetamide impurity in Step 5b processes.

In some embodiments, the solvent is DCM, IPAc, or MeOH. In some embodiments, the solvent is DCM or IPAc. In some embodiments, the solvent is DCM. In some embodiments, the solvent is IPAc.

In some embodiments, the reaction is maintained at a temperature of about 20-25° C. In some embodiments, the reaction is agitated for at least about 15, 16, 17, or 18 hours.

In some embodiments, the reaction reduces formation of one or more impurities such as TG Amine DesAc, di-NAG-OH TG Amine, TG Amine guanidyl, TG Amine Ac, TG Acetamide, and/or NAG-H guanidine, each of which are shown below.

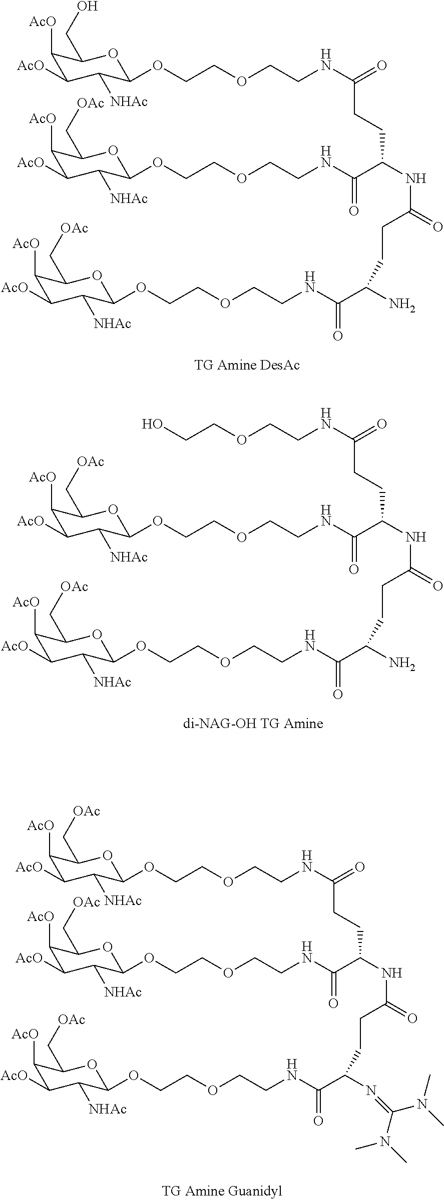

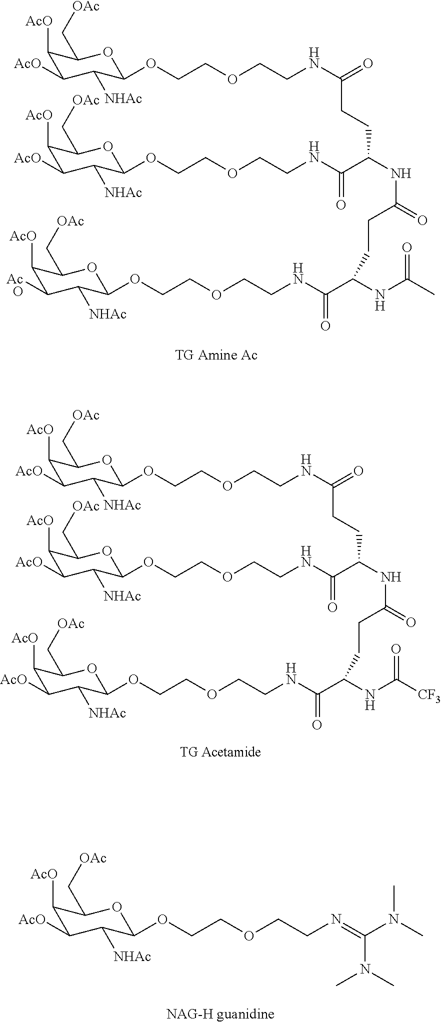 [SMILES: CC(=O)NC1C(OCCOCCNC(=O)CC[C@H](NC(=O)CC[C@H](NC(C)=O)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O ; CC(=O)NC1C(OCCOCCNC(=O)CC[C@H](NC(=O)CC[C@H](NC(=O)C(F)(F)F)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O ; CC(=O)NC1C(OCCOCCN=C(N(C)C)N(C)C)OC(COC(C)=O)C(OC(C)=O)C1OC(C)=O]

In some embodiments, the NAG-H guanidine impurities is present at least than or equal to 2 or 1% as measured by LC. In some embodiments, the TG Amine di-NAG-OH impurities is present at least than or equal to 2 or 1% as measured by LC. In some embodiments, the reaction reduces formation of TG Amine des-acyl impurities. In some embodiments, the TG Amine des-acyl impurities is present at least than or equal to 5, 4, 3, 2, 1% as measured by LC.

In some embodiments, the TG Amine or salt thereof is a TG Amine acid salt. In some embodiments, the TG Amine acid salt is a phosphate, formate, acetate, trifluoroacetate, or oxalate salt. In some embodiments, the TG Amine acid salt is trifluoroacetate or oxalate salt. In some embodiments, the TG Amine acid salt is trifluoroacetate salt.

The improved step 5a process leads to high purity of the TG PEG product of Step 5b while reducing the amount of a TG acetamide side product. In some embodiments, the TG Amine or salt thereof comprises less than about 10%, 9%, 8%, 7%, 6%, 5%, 4%, 3%, or 2% acetamide side product. In some embodiments, the TG Amine or salt thereof comprises less than about 5% acetamide side product. In some embodiments, the TG Amine or salt thereof comprises less than about 2% acetamide side product.

In some embodiments, the TG Amine or salt thereof produced from the improved step 5a process has greater than or equal to about 97%, 98%, or 99% purity as measured by LC. In some embodiments, the TG Amine or salt thereof produced from the improved step 5a process has greater than or equal to about 97, 98, or 99% yield.

In some embodiments, a step 5a process for preparing the TG Amine TFA salt is shown in the below scheme:

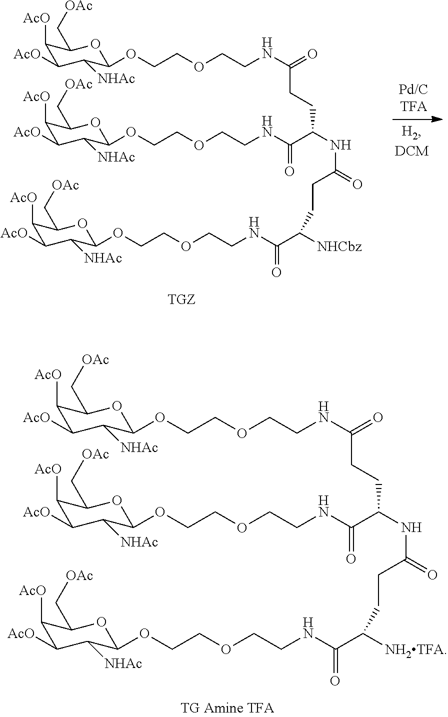

In some embodiments, a step 5a process for preparing the TG Amine TFA salt is shown in the below scheme:

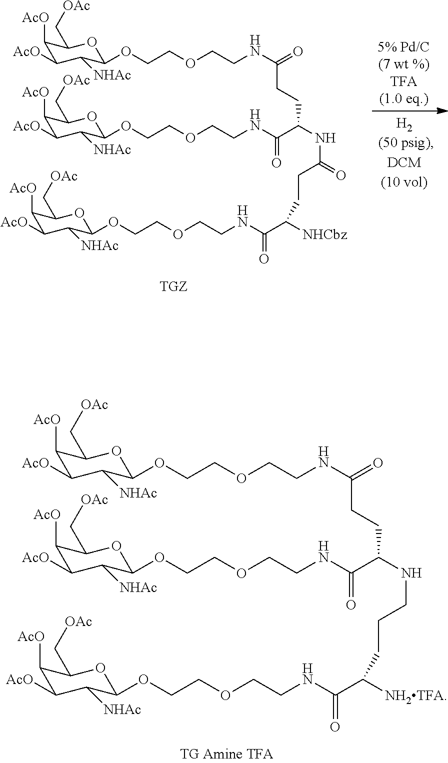

Following reaction and filtration, the resulting TG Amine or salt thereof in solvent can be directly telescoped to the step 5b processes described herein. The advantages include elimination of unit operations involved with distillation and precipitation. In some embodiments, the TG Amine or salt thereof is telescoped into Step 5b without isolation of the TG Amine or salt thereof. In some embodiments, the TG Amine or salt thereof is a salt that is TG Amine TFA.

### Step 5b: TG PEG

Disclosed herein are proceses for preparing TG PEG (step 5b processes). TG Amine or salt thereof is coupled with PEG acid to prepare TG PEG:

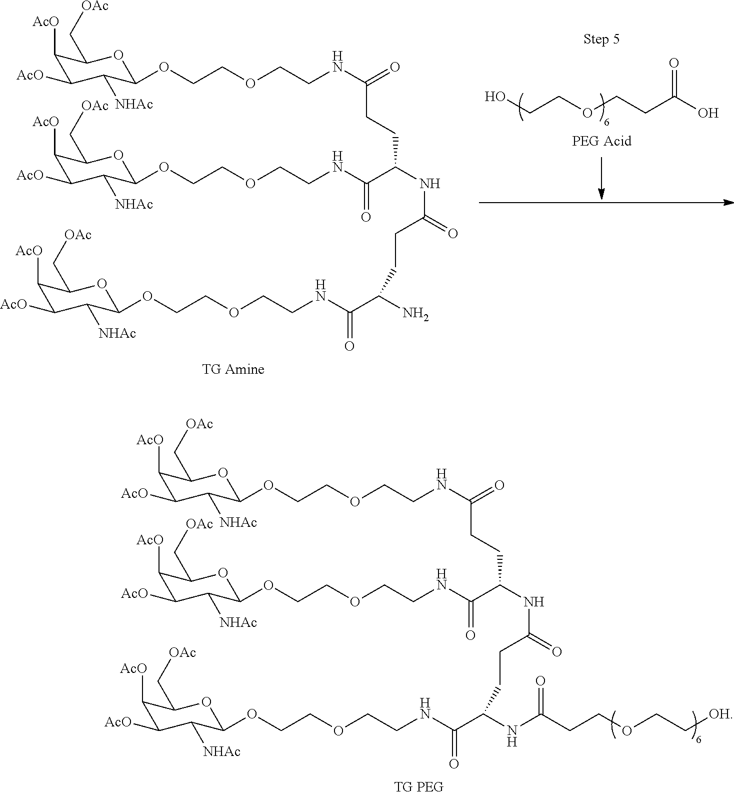

The improvements reduce impurities during the amide coupling process, eliminates column chromatography, and reduces solvent volumes. The improved process comprises controlled addition of coupling reagent (e.g., TBTU). By controlling the rate of coupling reagent (e.g., TBTU) addition to the PEG acid, the improved process reduces formation of a PEG-dimer impurity called TG PEG dimer. Additionally, improved step 5b comprises an extraction of crude TG PEG product into an aqueous water solution with subsequent back extraction for an orthogonal impurity rejection.

The improved step 5b processes comprising reacting TG Amine, or salt thereof, with PEG acid in the presence of a coupling reagent. In some embodiments, the process further comprises a base. In some embodiments, the process further comprises solvent. In some embodiments, the coupling reagent that is TBTU or HATU. In some embodiments, the coupling reagent is TBTU. In some embodiments, the solvent is DCM or DMF. In some embodiments, the solvent is DCM. In some embodiments, the coupling reagent is TBTU and the solvent is DCM. In some embodiments, the coupling reagent is HATU and the solvent is DMF.

In some embodiments, the step 5b process comprises adding TBTU to a solution comprising TG Amine or a salt thereof and PEG acid.

In some embodiments, step 5b comprises:
a) combining TG Amine or salt thereof with PEG acid;
b) adding a base; and
c) adding TBTU over a period of time. In some embodiments, the TG amine or salt thereof is directly added from the end of Step 5a processes. In some embodiments of step 5b, the process further comprises extraction/work-up, distillation, precipitation, filtration, and/or drying.

TBTU can be added portion-wise or using a slurry transfer. In some embodiments, the TBTU addition to a solution of PEG acid and the TG Amine or salt thereof is over a time period of about 80-100 or 90 minutes. In some embodiments, the TBTU addition to a solution of PEG acid and the TG Amine or salt thereof is over a time period of about 50-70 or 60 minutes.

In some embodiments, the step 5b process further comprises the use of a base. In some embodiments, the base is N,N-Diisopropylethylamine (DIPEA), triethylamine, or N-methylmorpholine (NMM). In some embodiments, the base is N,N-Diisopropylethylamine (DIPEA).

The improved process of preparing TG PEG reduces at least one impurities such as the TG PEG dimer impurity. During NAG-25 conjugation to an oligonucleotide, this TG PEG dimer impurity is reactive in a phosphitylation step and may conjugate to the oligonucleotide. Examples of impurities are shown below:

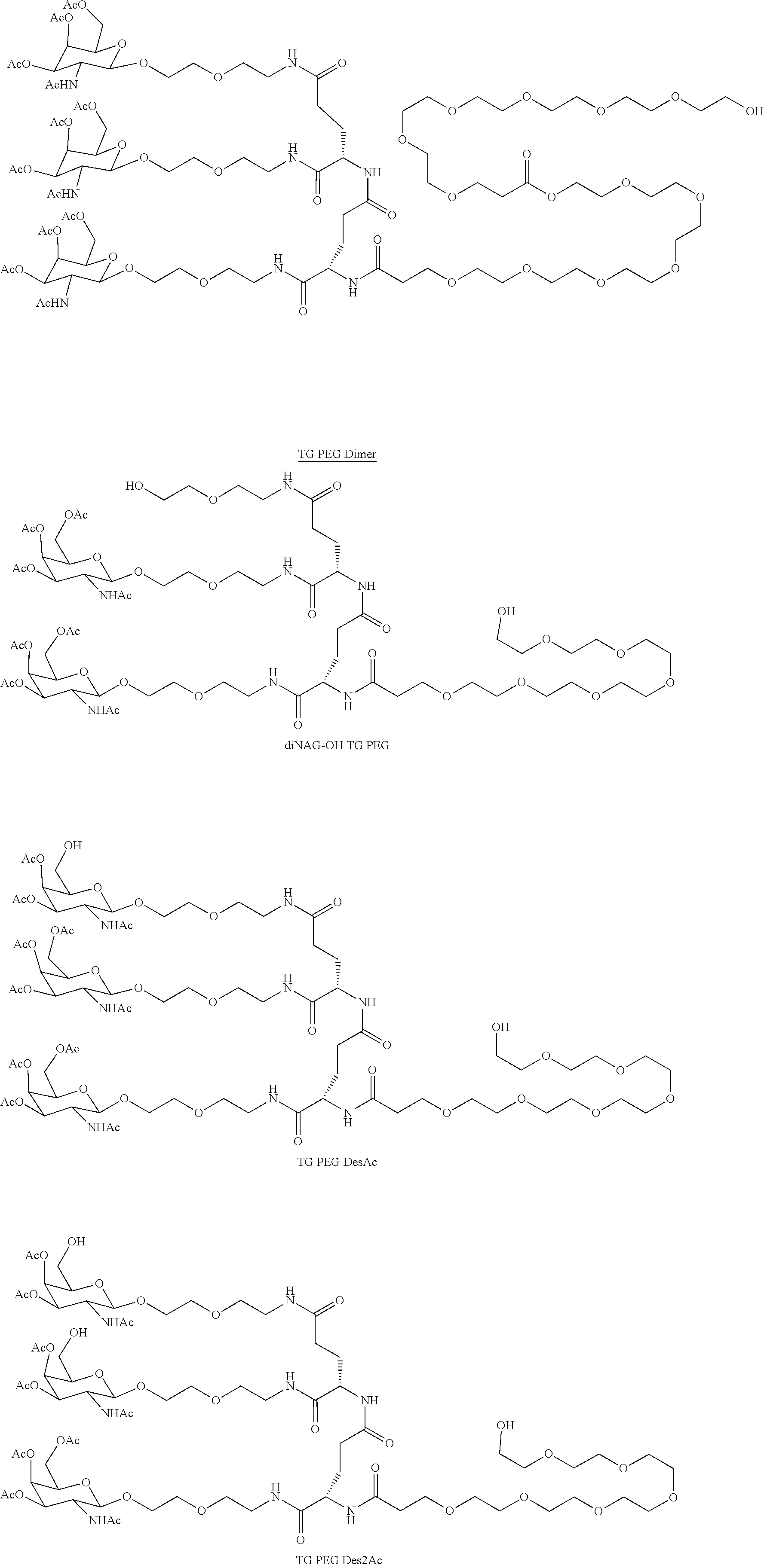

In some embodiments, the step 5b process produces TG PEG comprising less than 10, 9, 8, 7, 6, 5, 4, 3, 2, or 1% of TG PEG dimer. Disclosed are compositions comprising TG PEG with TG PEG dimer that is less than or equal to 10% (e.g., 10, 9, 8, 7, 6, 5, 4, 3, 2, 1%) as measured by LC.

In some embodiments, the TG Amine or salt thereof is TG Amine TFA salt. In some embodiments, a step 5b process of preparing TG PEG comprises reacting TG Amine TFA salt with PEG acid, TBTU, and DIPEA in a solvent. In some embodiments, the solvent is DCM, DMF, DMAc, and MeCN. In some embodiments, the solvent is DCM. In some embodiments, the solvent is DMF. In some embodiments, the step 5b process starts at about −10° C. to about 5° C. and is increased to about 17-27° C. over the course of the reaction. In some embodiments, the step 5b process starts at about −10° C. to about −4° C. and increased to about 17-27° C. over the course of the reaction. In some embodiments, the step 5b process starts at about −8° C. and is increased to about 17-27° C. over the course of the reaction. In some embodiments, the step 5b process starts at about −8° C. and is increased to room temperature over the course of the reaction.

In some embodiments, a step 5b process for preparing TG PEG is shown in the below scheme:

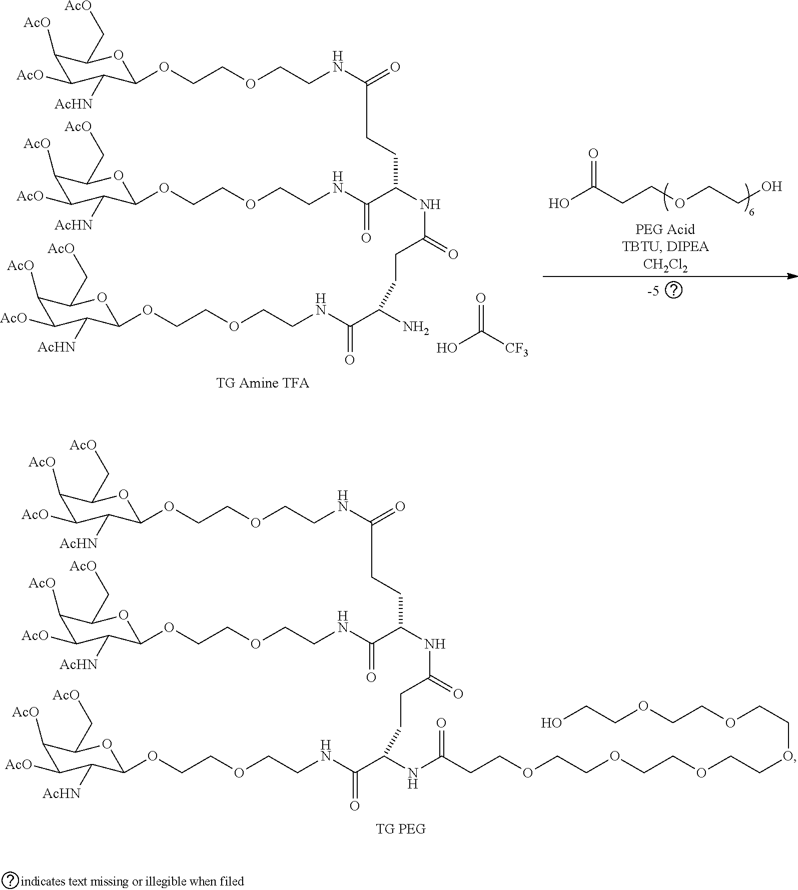

wherein TG Amine is a TG Amine TFA salt.

In some embodiments, a step 5b process for preparing TG PEG comprises combining a TG Amine with PEG Acid. In some embodiments, the process comprises DCM as solvent. In some embodiments, the temperature is maintained at about 20-25° C. In some embodiments, the temperature is cooled to −5 to 5° C., DIPEA is added, and then TBTU is added. In some embodiments, TBTU is added portion-wise or as a slurry over a period of time of about 60-90 minutes. In some embodiments, the reaction temperature is then increased slowly to about 0-5° C. over the period of about 1 hour. In some embodiments, the reaction is further maintained at 0-5° C. for about 1-5 hours. In the foregoing embodiments, PEG acid is added at about 0.9-1.3 eq, TBTU is added at about 1-1.5 eq, DIPEA is added at about 2.5-3.5 eq, each of which are relative to TG Amine in solution (from Step 5a). In the foregoing embodiments, PEG acid is added at about 1.1 eq, TBTU is added at about 1.3 eq, DIPEA is added at about 3.0 eq, each of which are relative to TG Amine in solution (from Step 5a). Additional amounts of PEG Acid and TBTU can optionally be added to increase reaction conversion.

Following reaction completion, crude TG PEG product is extracted into an aqueous water solution with subsequent back extraction to remove one or more impurities. In some embodiments of step 5b, the process further comprises extraction/work-up, distillation, precipitation, filtration, and/or drying.

In some embodiments, the step 5b process comprises an aqueous extraction wash sequence for rejecting the TG PEG dimer impurity. In some embodiments, the aqueous extraction wash sequence comprises water (e.g., DI water) extraction, ammonium sulfate salt out (e.g., 13 wt % ammonium sulfate), back extraction into solvent (e.g., DCM back extraction), acidic buffer wash (e.g., pH 4-6), and basic buffer wash (e.g., pH 7.5-9).

In some embodiments, the acidic buffer wash comprises a mixture of 0.5M sodium monophosphate pH 6.0 and saturated brine. In some embodiments, the ratio of NaH₂PO₄:brine is about 4.5-5 volumes: 3-3.5 volumes. In some embodiments, the ratio is about 4.8 vol:3.2 vol.

In some embodiments, the basic buffer wash comprises a mixture of saturated sodium bicarbonate and saturated brine solution. In some embodiments, the ratio of NaHCO₃:brine is about 3.5-4.5 volumes:3.5-4.5 volumes. In some embodiments, the ratio is 4.0 vol:4.0 vol.

Following the aqueous extraction wash sequence, the TG PEG solution comprises TG PEG that is at least 90% pure and one or more impurities (e.g., diNAG-OH TG PEG, TG PEG DesAc, TG PEG Dimer, TG PEG Des2Ac, TG Amine Ac, TG acetamide, and/or TG Amine guanidyl) at less than 1% for each impurity.

In some embodiments, step 5b process comprises azeotropic distillation with a solvent. In some embodiments, the solvent for distillation is DCM.

Following washes and distillation, the process additionally comprises precipitation by adding antisolvent to the mixture. In some embodiments, the antisolvent is MTBE or IPAc. In some embodiments, the antisolvent is MTBE. In some embodiments, the precipitation occurs at a temperature of about −5-5° C.

In some embodiments, the step 5b process produces TG PEG in greater than or equal to about 90% or 95% purity as measured by LC. In some embodiments, the step 5b process produces TG PEG in greater than or equal to about 80, 81, 82, 83, 84, 85, or 90% yield.

### Step 6: NAG-25

Disclosed herein is a process of preparing NAG-25 (step 6 processes). The processes comprise phosphorus amide formation from TG PEG to prepare NAG-25:

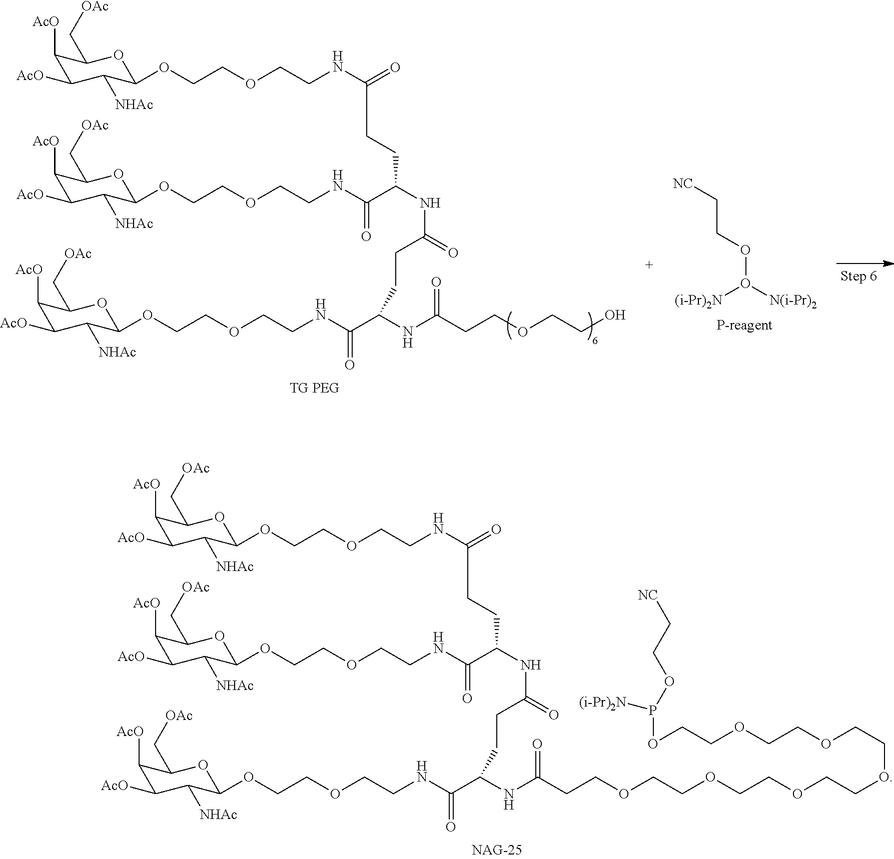

Improvements comprise changing precipitation solvent, which greatly reduced solvent volumes from a prior process (e.g., from 130-170 volumes to about 10-20, 20-30, 25-35, 30-50, or 40-60 volumes), increased NAG-25's stability, and/or reduced overall impurities (e.g., from 90-93 or 92% purity to about 95-98 or 97% purity).

In some embodiments the process of preparing NAG-25 comprises combining TG PEG, activator, and P-reagent. In some embodiments, the phosphitylating reagent (“P-reagent”) is 2-cyanoethyl-N, N, N′, N′-tetraisopropylphosphorodiamidite or 2-Cyanoethyl N,N-diisopropylchlorophosphoramidite. In some embodiments the phosphitylating reagent is 2-cyanoethyl-N, N, N′, N′-tetraisopropylphosphorodiamidite.

In some embodiments, the activator is tetrazole, 4,5-dicyanoimidazole (DCI), 5-Ethylthio-1H-Tetrazole (ETT), or Benzothiotetrazole (BTT). In some embodiments the activator is tetrazole, DCI, or ETT. In some embodiments the activator is tetrazole or DCI.

In some embodiments, the activator is tetrazole. In some embodiments the tetrazole is added in about 0.2-1.2 eq. In some embodiments the tetrazole is added in about 0.4-0.8 eq. In some embodiments the tetrazole is added in about 0.6 eq. The equivalents of tetrazole in the foregoing embodiments are relative to TG PEG.

In some embodiments the activator is DCI. In some embodiments the DCI is added in about 0.01-0.05 eq. In some embodiments the DCI is added in about 0.02-0.04 eq. In some embodiments the DCI is added in about 0.02-0.03 eq. The equivalents of DCI in the foregoing embodiments are relative to TG PEG.

In some embodiments step 6 reaction further comprises an additive. In some embodiments, step 6 optionally comprises adding an additive to TG PEG. In some embodiments, the additive is N-methylimidazole (NMI). In some embodiments step 6 reaction not comprise an additive.

In some embodiments, the P-reagent is added in about 0.75-2 eq. In some embodiments, the P-reagent is added in about 1-1.5 eq. The equivalents in the foregoing embodiments are relative to TG PEG.

Improvements to the process includes order of addition of the reagents. In some embodiments, a solution comprising P-reagent and activator is added to a solution of TG PEG that optionally comprises additive. The P-reagent with activator solution is added at about 0-5° C. After addition is complete, the reaction mixture is warmed to room temperature (e.g., 20-25° C.) and allowed to proceed for about 2-4 hours.

In some embodiments, a solution of TG PEG in solvent (a TG PEG solution) is added to another solution of P-reagent with activator in solvent. The TG PEG solution can be added over a period of time.

In some embodiments, the period of time is about 2-10, 3-7, 3-5, 4-5, 4-6, or 5-6 hours or about 3, 4, 5, 6, 7 hours. In some embodiments, the reaction proceeds at a temperature of about 35-45° C. or about 38, 39, 40, 41, 42, 43, 44, or 45° C. In some embodiments, the reaction proceeds for about 8-12, 9-11, or 10-14 hours or at least 8, 9, 10, 11, 12, 13 or 14 hours. In some embodiments, the P-reagent is 2-cyanoethyl-N, N, N′, N′-tetraisopropylphosphorodiamidite. In some embodiments, the activator is DCI. In some embodiments, the solvent is DCM. In some embodiments, the solvent is anhydrous DCM. The amount of equivalents P-reagent, activator have been described in prior embodiments. In some embodiments, a solution of TG PEG in DCM is added to another solution of 2-cyanoethyl-N, N, N′, N′-tetraisopropylphosphorodiamidite with DCI in DCM (e.g., anhydrous DCM) over a 4.5-5.5 hour (e.g., about 5 hours) time period and then allowed to react for at least 10 hours at 35-45° C. (e.g., about 40° C.). In some embodiments, the process comprises 1-1.5 eq of 2-cyanoethyl-N, N, N′, N′-tetraisopropylphosphorodiamidite, 0.02-0.03 eq of DCI, each relative to TG PEG.

The improved step 6 process further comprises the ability to identify, control, and/or reduce reaction impurities compared to other NAG-25 processes. The improved step 6 processes also comprises rejecting reactive phosphorus related impurities. Examples of impurities from Step 6 include NAG-25 dimer:

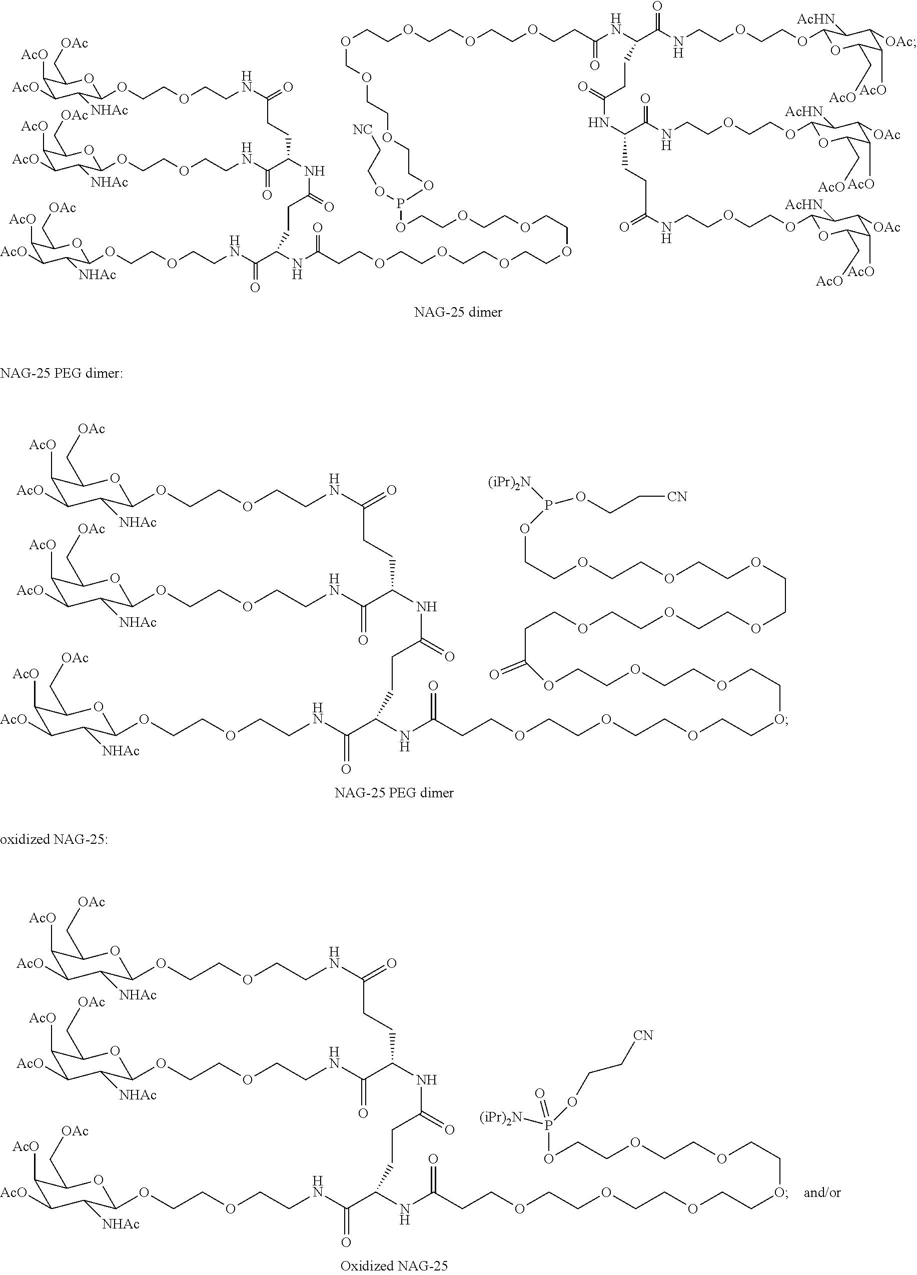

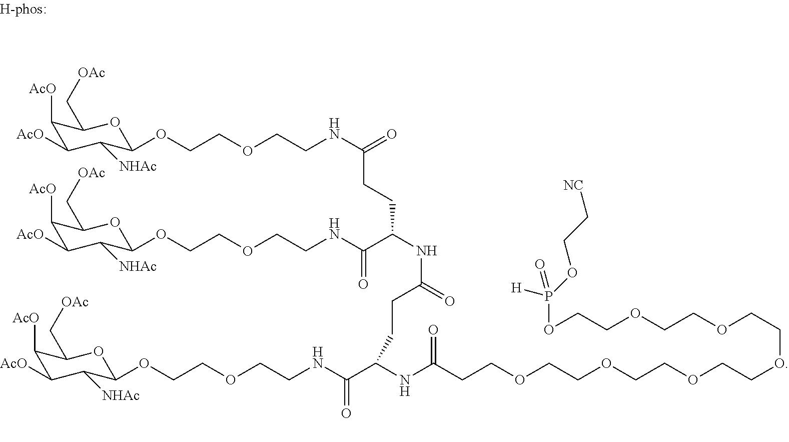 [SMILES: CC(=O)NC1C(OCCOCCNC(=O)CC[C@H](NC(=O)CC[C@H](NC(=O)CCOCCOCCOCCOCCOCCOCCO[PH](=O)OCCC#N)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]

In some embodiments, the impurities are H-phos, oxidized NAG-25, NAG-25 dimer, and/or NAG-25 PEG dimer. In some embodiments, the H-phos, oxidized NAG-25, NAG-25 dimer, and/or NAG-25 PEG dimer that is present at the end of reaction at an amount of less than or equal to about 15, 14, 13 12, 11, 10, 9, 8, 7, 6, 5, 4, 3, 2, or 1% as measured by LC. In some embodiments, the H-phos, oxidized NAG-25, NAG-25 dimer, and/or NAG-25 PEG dimer that is present at the end of reaction at an amount of less than or equal to about 8, 7, 6, 5, 4, 3, 2, or 1% as measured by LC. In some embodiments, the NAG-25 dimer that is present at the end of reaction at an amount of less than or equal to about 10, 9, 8, 7, 6, 5, 4, 3, 2, or 1% as measured by LC. In some embodiments, the NAG-25 dimer that is present at the end of reaction at an amount of less than or equal to about 5, 4, 3, 2, or 1% as measured by LC. In some embodiments, the NAG-25 dimer that is present at the end of reaction at an amount of less than or equal to about 4 or 3% as measured by LC. In some embodiments, the NAG-25 dimer that is present at the end of reaction at an amount of less than or equal to about 2 or 1% as measured by LC.

Disclosed are compositions comprising NAG-25 with any of the impurities described that is less than or equal to 10% (e.g., 10, 9, 8, 7, 6, 5, 4, 3, 2, 1%) as measured by LC. Disclosed are compositions comprising NAG-25 with NAG-25 dimer that is less than or equal to about 10, 9, 8, 7, 6, 5, 4, 3, 2, or 1% as measured by LC. In some embodiments, the composition comprises NAG-25 with NAG-25 dimer that is less than or equal to 5, 4, 3, 2, or 1% as measured by LC.

In some embodiments Step 6 process further comprises an extraction (e.g., water extractions), distillation, precipitation, a filtration step, and/or a drying step.

In some embodiments, NAG-25 product solution is dried. In some embodiments, the drying step uses molecular sieves. In some embodiments, the drying step uses azeotropic distillation.

In some embodiments, precipitation step uses normal precipitation, wherein antisolvent is added to the NAG-25 solution. In some embodiments, precipitation step uses inverse precipitation, wherein the NAG-25 solution is added to antisolvent.

In some embodiments, the antisolvent used for precipitation is MTBE or heptane. In some embodiments, the antisolvent used for precipitation is heptane.

In some embodiments, NAG-25 is precipitated from DCM/heptane as the solvent/antisolvent.

In some embodiments, the step 6 process comprises reacting TG PEG with 2-cyanoethyl-N, N, N′, N′-tetraisopropylphosphorodiamidite in the presence of tetrazole and NMI in dichloromethane.

In some embodiments, the step 6 process is shown in the scheme below:

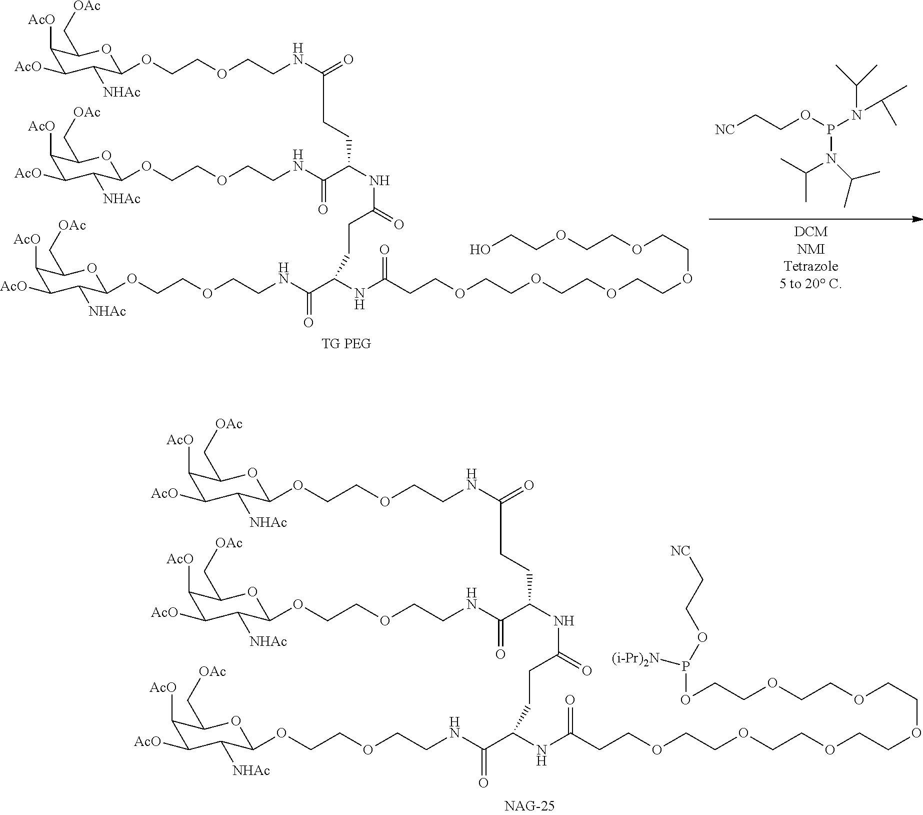

In some embodiments of the above scheme, DCM is about 15V, NMI is about 0.2 eq, tetrazole is about 0.6 eq, and the P-reagent is about 1.25 eq. In some embodiments, the TG PEG solution is added to the P-reagent with activator solution in a period of time that is about 2-10 hours and temperature that is about 35-45° C.

In some embodiments of step 6, the process further comprises extraction/work-up, crystallization, precipitation, filtration, and/or drying. In some embodiments of step 6, NAG-25 extraction with water washes are optional.

In some embodiments, the step 6 process comprises a solution of TG PEG in solvent that is added to a solution of P-reagent with activator in solvent. In some embodiments, the P-reagent is 2-cyanoethyl-N, N, N′, N′-tetraisopropylphosphorodiamidite. In some embodiments the process does not comprise an additive. In some embodiments, the solvent is DCM. In some embodiments, the activator is DCI. The TG PEG solution is added over a period of time to the P-reagent with activator solution. In some embodiments, the period of time is about 4-6 hours, 4.75-5.25 hours, or 4.5-5.5 hours. Following the addition of the TG PEG solution, the two solutions are allowed to react for at least 8-12 hours. In some embodiments, the time to react is about 9-11 hours. In some embodiments, the reaction is carried out at about 35-45° C. In some embodiments, the reaction is carried out at about 38-42° C. After the reaction was completed, aqueous extractions with water can optionally be performed and NAG-25 is azeotropically dried (e.g., at 25-35° C. under vacuum), followed with filtration. In some embodiments, NAG-25 is precipitated without aqueous extraction. For precipitation, the NAG-25 solution can be added to anhydrous antisolvent (e.g., heptane) over 90 minutes. The solids are filtered, washed twice with antisolvent:solvent (e.g., anhydrous heptane:DCM). In some embodiments, the antisolvent:solvent ratios are about 4:1, 3:1, 2:1, or 1:1. In some embodiments, the heptane:DCM ratios are about 4:1, 3:1, 2:1, or 1:1. In some embodiments, the heptane:DCM ratios are about 4:1 or 3:1. In some embodiments, the volume percent of solvent in the antisolvent:solvent solution is about 20-50, 20-30, 30-40, or 40-50%, with the remaining percentage being made up the antisolvent volume percent. In some embodiments, the percentage of DCM is about 20-50% or about 20, 30, 40, 50% of the volume of the antisolvent/solvent solution.

### Linking to Oligomeric Compounds

NAG-25 and NAG-25-containing compounds are useful as targeting ligands by linking to therapeutic compounds, such as an oligomeric compound (e.g. RNA). NAG-25 facilitates targeted delivery of the therapeutic compounds to hepatocytes for endocytosis of the NAG-25-conjugated therapeutic compound, wherein the therapeutic compound can modulate expression of a target nucleic acid resulting in altered translation of a target nucleic acid. For example, the therapeutic compound can modulate expression of a target gene to inhibit protein translation or expression.

In some embodiments, the targeting ligand is linked to the therapeutic compound via an additional linker and/or a cleavable moiety, which is then linked to the therapeutic compound. In some embodiments, targeting ligands are ligated to the therapeutic compound itself.

In some embodiments, the therapeutic compound is an expression-inhibiting oligomeric compound. In some embodiments, the expression-inhibiting oligomeric compound is an RNAi construct. In some embodiments, the expression-inhibiting oligomeric compound is a double-stranded RNAi construct. In some embodiments the expression-inhibiting oligomeric compound is a single-stranded oligonucleotide. The expression-inhibiting oligomeric compounds may be synthesized using methods commonly used in the art.

In some embodiments, the targeting ligand is linked directly or indirectly to the 5′ end of the sense strand of a double-stranded RNAi construct. In some embodiments, the targeting ligand is linked directly or indirectly to the 3′ end of the sense strand of a double-stranded RNAi construct. In some embodiments, the targeting ligand is linked directly or indirectly to the 5′ end or the 3′ end of the antisense strand of a double-stranded RNAi construct. In some embodiments, the targeting ligand is linked directly or indirectly to the 5′ end or the 3′ end of a single-stranded RNAi construct.

In some embodiments, a targeting ligand is linked to a double-stranded RNAi construct via a phosphate, phosphonate, phosphorothioate, or other internucleoside linking group, at the 5′ end of the terminal nucleoside of the sense strand of the double-stranded RNAi construct.

In some embodiments, a targeting ligand disclosed herein includes a cleavable moiety. In some embodiments, a cleavable moiety includes or consists of a phosphate or other internucleoside linking group that may be cleaved. In some embodiments, the targeting ligand is linked to a therapeutic compound via a cleavable moiety.

In some embodiments, a targeting ligand disclosed herein is linked to an additional group or groups that includes a cleavable moiety. In some embodiments, the targeting ligand is linked to a cleavable moiety, which is then linked to an expression-inhibiting oligomeric compound.

In some embodiments, the targeting ligand is a phosphoramidite compound (also referred to herein as a “phosphoramidite-containing compound”). A phosphoramidite compound including a targeting ligand described herein may be useful to readily attach the targeting ligand to the therapeutic compound or to other groups, using methods generally known in the art for phosphoramidite synthesis. In some embodiments, the phosphoramidite compound including the targeting ligand is linked to an expression-inhibiting oligomeric compound using methods generally known in the art. In some embodiments, the targeting ligand-containing phosphoramidite is linked to the 5′ end of the sense strand of a double-stranded RNAi construct.

In some embodiments, an expression-inhibiting oligomeric compound linked to a targeting ligand includes a single-stranded oligonucleotide. In some embodiments, the single-stranded oligonucleotide is a single-stranded antisense oligonucleotide. In some embodiments, the targeting ligand is linked directly to a single-stranded antisense oligonucleotide. In some embodiments, additional groups are inserted between a targeting ligand and a single-stranded oligonucleotide.

In some embodiments, an expression-inhibiting oligomeric compound linked to any of the targeting ligands disclosed herein includes an RNAi construct. In some embodiments, a targeting ligand disclosed herein is linked, either directly or indirectly, to an RNAi construct.

In some embodiments, a targeting ligand disclosed herein is linked directly to an RNAi construct. In some embodiments, a targeting ligand disclosed herein is linked indirectly to an RNAi construct, as additional group(s) are inserted between the RNAi construct and the linker of the targeting ligand. In some embodiments, a second linker is included between the linker and the therapeutic compound (e.g., RNAi construct).

### OTHER EMBODIMENTS

A listing of exemplary embodiments includes:

1. A process for preparing t-Butyl Core

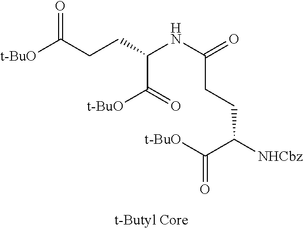 [SMILES: CC(C)(C)OC(=O)CC[C@H](NC(=O)CC[C@H](NC(=O)OCc1ccccc1)C(=O)OC(C)(C)C)C(=O)OC(C)(C)C]

said process comprising reacting GluZ:

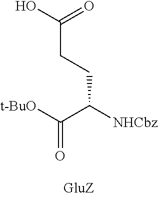 [SMILES: CC(C)(C)OC(=O)[C@H](CCC(=O)O)NC(=O)OCc1ccccc1]

with GluOtBu or salt thereof:

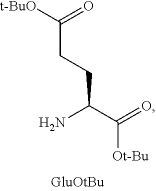 [SMILES: CC(C)(C)OC(=O)CC[C@H](N)C(=O)OC(C)(C)C]

to produce t-Butyl Core.
2. The process of embodiment 1 wherein GluOtBu or salt thereof is a GluOtBu hydrochloride salt:

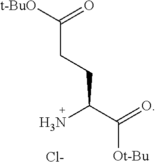 [SMILES: CC(C)(C)OC(=O)CC[C@H](N)C(=O)OC(C)(C)C ; [Cl-]]

3. The process of embodiment 1 or 2, wherein said reacting is performed in the presence of a coupling reagent, a base, and a solvent.
4. The process of any one of embodiments 1-3 wherein the coupling reagent is EDC/Oxyma, TFFH, PyOxim, CDI, PivCl, T3P, or COMU.
5. The process of any one of embodiments 1-4 wherein the coupling reagent is T3P or PivCl.
6. The process of any one of embodiments 1-5 wherein the couple reagent is PiVCl.
7. The process of any one of embodiments 1-6 wherein the base is N-methylmorpholine (NMM).
8. The process of any one of embodiments 1-7 wherein the solvent is IPAc, MeTHF, MIBK, or MTBE.
9. The process of any one of embodiments 1-8 wherein the solvent is MTBE.
10. The process of any one of embodiments 1-9 wherein the antisolvent is heptane.
11. The process of any one of embodiments 1-10 wherein the solvent is MTBE and the antisolvent is heptane.
12. The process of any one of embodiments 1-11 wherein a first solution comprising GluZ and NMM in a solvent is added to a second solution comprising the solvent and PivCl.
13. The process of any one of embodiments 1-12, PivCl is in excess.
14. The process of any one of embodiments 1-13, wherein GluOtBu is in excess.
15. The process of any one of embodiments 1-14, wherein conversion to t-Butyl Core is greater than about 90%, based on the amount of GluZ.
16. A process of preparing Triacid:

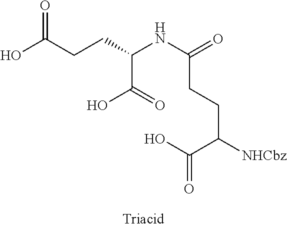 [SMILES: O=C(O)CC[C@H](NC(=O)CCC(NC(=O)OCc1ccccc1)C(=O)O)C(=O)O]

said process comprising reacting t-Butyl core with an acid.
17. The process of embodiment 16 wherein the acid is phosphoric acid (H₃PO₄), TFA, HCl, benzenesulfonic acid, or p-toluenesulfonic acid.
18. The process of embodiment 16 wherein the acid is phosphoric acid (H₃PO₄).
19. The process of any one of embodiments 16-18 wherein the reaction is conducted in a solvent selected from 2-MeTHF, acetonitrile, THF, DMA, sulfolane, and DME.
20. The process of embodiment 19 wherein the solvent is 2-MeTHF, THF, or acetonitrile.
21. The process of embodiment 19 wherein the solvent is 2-MeTHF or THF.
22. The process of embodiment 19 wherein the solvent is 2-MeTHF.
23. The process of any one of embodiments 16-22 wherein the Triacid is isolated as a crystalline solid.
24. The process of any one of embodiments 16-23 wherein the Triacid has greater than or equal to 95% purity as measured by LC.
25. The process of any one of embodiments 16-24 wherein the solvent further comprises water.
26. The process of any one of embodiments 16-25 wherein the solvent is a mixture of 2-MeTHF and water.
27. The process of any one of embodiments 16-26 wherein about 3.0-4.0 vol of 2-MeTHF and about 0.5 2 vol water is used.
28. The process of any one of embodiments 16-27 wherein reaction is conducted at a temperature of about 40-60° C.
29. The process of any one of embodiments 16-28 wherein reaction is conducted at a temperature of about 45-55° C.
30. The process of any one of embodiments 16-29 wherein reaction is conducted at a temperature of about 50° C.
31. The process of any one of embodiments 16-30 further comprising removing phosphoric acid with at least one organic/aqueous wash.
32. The process of any one of embodiments 16-31 wherein the organic/aqueous wash comprises isopropylacetate (iPAC) as organic layer and ammonium sulfate as aqueous layer.
33. The process of any one of embodiments 16-32 wherein about 8-18 vol of iPAC and about 3-7 vol 20 wt % of ammonium sulfate are used.
34. The process of any one of embodiments 16-33 wherein about 10 vol of iPAC and about 5 vol 20 wt % of ammonium sulfate are used.
35. The process of any one of embodiments 16-34 further comprising crystallization of Triacid using acetone/toluene, 2-MeTHF/IPAc, 2-MeTHF/CPME, acetone/heptane, or MeTHF/acetonitrile.
36. The process of any one of embodiments 16-35 further comprising crystallization of Triacid using acetone/toluene.
37. The process of any one of embodiments 16-36 wherein the acetone/toluene is in ratio of 2:3, 1:1, 3:2, or 1:2.
38. The process of any one of embodiments 16-37 wherein the acetone/toluene is in ratio of 2:3, 1:1, or 1:2.
39. The process of any one of embodiments 16-38, wherein the Triacid is obtained in a crystalline form characterized by an X-ray powder diffractogram having at least a signal at three two-theta values chosen from 7.4±0.2, 9.2±0.2, 21.0±0.2, 14.6±0.2, 18.3±0.2, and 19.3±0.2.
40. A crystalline Form I of Triacid, characterized by an X-ray powder diffractogram having at least a signal at three two-theta values selected from 7.4±0.2, 9.2±0.2, 21.0±0.2, 14.6±0.2, 18.3±0.2, and 19.3±0.2.
41. A crystalline Form I of Triacid, characterized by an X-ray powder diffractogram having a signal at two-theta values of 7.4±0.2, 9.2±0.2, and 21.0±0.2.
42. A crystalline Form I of Triacid, characterized by an X-ray powder diffractogram substantially similar to that in FIG. 2.
43. A crystalline Form II of Triacid, characterized by an X-ray powder diffractogram substantially similar to that in FIG. 3.
44. A process of preparing TGZ

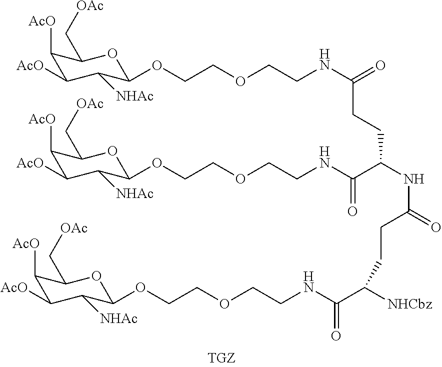 [SMILES: CC(=O)NC1C(OCCOCCNC(=O)CC[C@H](NC(=O)CC[C@H](NC(=O)OCc2ccccc2)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]

said process comprising reacting Triacid with NAG-H:

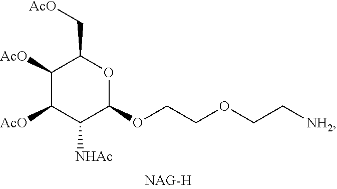 [SMILES: CC(=O)N[C@H]1[C@H](OCCOCCN)O[C@H](COC(C)=O)[C@H](OC(C)=O)[C@@H]1OC(C)=O]

in the presence of a coupling reagent and a base.
45. The process of embodiment 44 wherein the coupling reagent is TBTU, HATU or TCFH.
46. The process of embodiment 45 wherein the coupling reagent is TBTU.
47. The process of any one of embodiments 44-46 wherein the base is DIPEA, NMI, NMM, or TMP.
48. The process of embodiment 47 wherein the base is NMI.
49. The process of any one of embodiments 44-48 further comprising at least one buffer wash.
50. The process of embodiment 49 wherein the buffer wash is a phosphate buffer.
51. The process of embodiments 49 or 50 wherein the buffer wash is about pH 5-7.
52. The process of embodiment 51 wherein the buffer wash is about pH 6.
53. The process of any one of embodiments 44-52, wherein said reacting is conducted in a solvent selected from DCM, DMF, MeCN, and DMAc, or combination thereof.
54. The process of embodiment 53 wherein the solvent is DMAc.
55. The process of embodiments 53 or 54 further comprising adding an anti-solvent to a solution comprising TGZ and the solvent.
56. The process of embodiment 55 wherein the anti-solvent is an ethereal solvent.
57. The process of embodiment 56 wherein the ethereal solvent is DME, 2-MeTHF, or MTBE.
58. The process of any one of embodiments 53-57 wherein the solvent is DCM and the anti-solvent is MTBE.
59. The process of any one of embodiments 44-58 wherein TGZ is prepared with reduced impurities.
60. The process of embodiment 59 wherein the impurity is des-acyl.
61. The process of any one of embodiments 44-60 further comprises precipitation of TGZ from solvent/antisolvent system.
62. The process of embodiment 61 wherein the solvent/antisolvent system is DCM/MTBE.
63. The process of any one of embodiments 44-62, wherein TGZ is obtained in at least 95% purity without use of column chromatography.
64. The process of any one of embodiments 44-63 wherein the NAG-H, or salt thereof, was carried forward as a solution from a prior reaction to react with Triacid without isolation of the NAG-H, or salt thereof, from the prior reaction.
65. The process of embodiment 64 wherein NAG-H, or salt thereof, is prepared in a prior reaction comprising comprising hydrogenation of NAG-Z the presence of a Pd/C catalyst and in dimethylacetamide (DMAc).
66. The process of any one of embodiments embodiment 44-65 wherein the DMAc is about 3 volumes to about 5 volumes.
67. The process of any one of embodiments embodiment 44-65 wherein the DMAc is about 4 volumes.
68. The process of any one of embodiments embodiment 44-67 wherein the NAG-H or salt thereof is NAG-H TFA.
69. The process of any one of embodiments 44-66 wherein the Pd/C catalyst is 5% Pd/C.
70. The process of any one of embodiments 44-69 wherein NAG-H TFA is prepared in a prior reaction comprising the reagents in the below scheme:

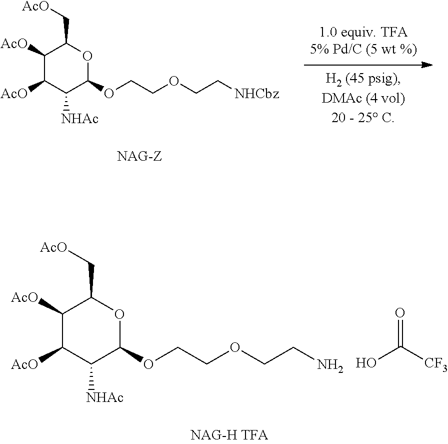 [SMILES: CC(=O)N[C@H]1[C@H](OCCOCCNC(=O)OCc2ccccc2)O[C@H](COC(C)=O)[C@H](OC(C)=O)[C@@H]1OC(C)=O ; CC(=O)N[C@H]1[C@H](OCCOCCN)O[C@H](COC(C)=O)[C@H](OC(C)=O)[C@@H]1OC(C)=O ; O=C(O)C(F)(F)F]

71. A process for preparing NAG-Z:

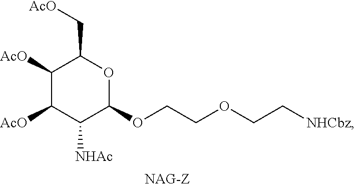 [SMILES: CC(=O)N[C@H]1[C@H](OCCOCCNC(=O)OCc2ccccc2)O[C@H](COC(C)=O)[C@H](OC(C)=O)[C@@H]1OC(C)=O]

said process comprising reacting Acyl GalNAc:

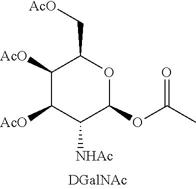 [SMILES: CC(=O)N[C@H]1[C@H](OC(C)=O)O[C@H](COC(C)=O)[C@H](OC(C)=O)[C@@H]1OC(C)=O]

with Alcohol-Z

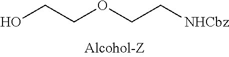 [SMILES: O=C(NCCOCCO)OCc1ccccc1]

to produce NAG-Z.
72. The process of embodiment 71, wherein the reaction is performed in the presence of acid.
73. The process of embodiment 72, wherein the acid is bismuth trifluoromethanesulfonate (Bi(OTf)₃), boron trifluoride etherate (BF₃OEt₂), tert-butyldimethylsilyl trifluoromethanesulfonate (TBSOTf), triisopropylsilyl trifluoromethanesulfonate (TIPSOTf), indium triflate (In(OTf)₃), or copper triflate (Cu(OTf)₂).
74. The process of embodiment 73, wherein the acid is bismuth trifluoromethanesulfonate (Bi(OTf)₃), boron trifluoride etherate (BF₃OEt₂), indium triflate (In(OTf)₃), or copper triflate (Cu(OTf)₂).
75. The process of any one of embodiments 72-74, wherein the acid is present at 0.1-0.2 equivalents, relative to Alcohol-Z.
76. The process of any one of embodiments 72-74, wherein the acid is present at 0.13-0.17 equivalents, relative to Alcohol-Z.
77. The process of any one of embodiments 71-76, wherein the reaction is performed in the presence of a solvent selected from acetonitrile, dichloromethane, and dichloroethane.
78. The process of embodiment 77, wherein the solvent is present at 8-12 volumes in total.
79. The process of any one of embodiments 71-78, wherein the Acyl GalNAc is present at 1.0-2.0 equivalents, relative to Alcohol-Z.
80. The process of embodiment 79, wherein the Acyl GalNAc is present at 1.3-1.7 equivalents, relative to Alcohol-Z.
81. The process of any one of embodiments 71-80, wherein the reaction temperature is heated to about 55-65° C. or 58-62° C.
82. The process of any one of embodiments 71-81, wherein the process comprises the reagents and conditions in the below scheme:

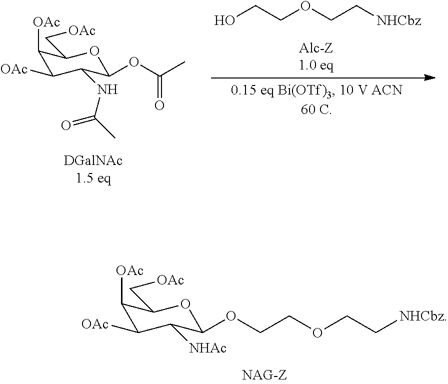 [SMILES: O=C(NCCOCCO)OCc1ccccc1 ; CC(=O)NC1C(OC(C)=O)OC(COC(C)=O)C(OC(C)=O)C1OC(C)=O ; CC(=O)NC1C(OCCOCCNC(=O)OCc2ccccc2)OC(COC(C)=O)C(OC(C)=O)C1OC(C)=O]

83. The process of any one of embodiments 71-82, wherein the Alcohol-Z is prepared by reacting benzyl chloroformate with aminoalcohol in presence of a solvent and a base.
84. The process of embodiment 83, wherein the solvent is dichloromethane (DCM).
85. The process of embodiment 83 or 84, wherein the base is triethylamine (TEA).
86. The process of any one of embodiments 83-85, wherein the aminoalcohol is present at 1.1 equivalents, relative to benzyl chloroformate.
87. The process of any one of embodiments 73-86, wherein the reaction temperature is about 30-40° C.
88. The process of embodiment 86, wherein the reaction temperature is about 33-37° C.
89. The process of any one of embodiments 77-87, wherein the solvent is acetonitrile and additional water is added to crystallize the NAG-Z.
90. A process of preparing TG Amine

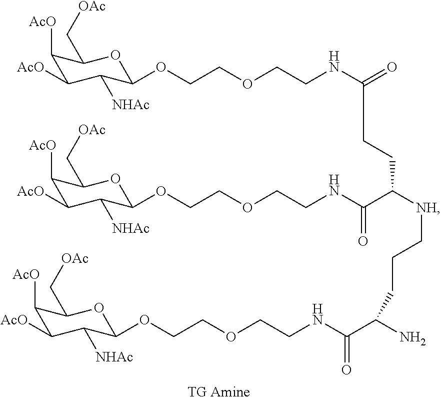 [SMILES: CC(=O)NC1C(OCCOCCNC(=O)CC[C@H](NCCC[C@H](N)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]

or a salt thereof,
said process comprising a high pressure hydrogenolysis for a carboxybenzyl deprotection of TGZ to form the TG Amine or a salt thereof.
91. The process of embodiment 90 comprising a palladium source for hydrogenolysis.
92. The process of embodiment 91 wherein the palladium source is palladium on carbon (Pd/C) catalyst.
93. The process of embodiment 92 wherein the palladium source is 5% Pd/C.4d. The process of any one of embodiments 4a-4c wherein the catalyst loading is less than about 10%, 9%, 8%, 7%, 6% Pd/C. In some embodiments, the catalyst used is 5% Pd/C.
94. The process of any one of embodiments 90-93, wherein said hydrogenolysis is performed in the presence of an acid.
95. The process of embodiment 94 wherein the acid is TFA, oxalic acid, HCl, AcOH, H₃PO₄, citric.
96. The process of embodiment 94 wherein the acid is TFA or oxalic acid.
97. The process of embodiment 94 wherein the acid is TFA.
98. The process of any one of embodiments 90-97 said hydrogenolysis is performed in a solvent, and the solvent is DCM, IPAc, or MeOH.
99. The process of embodiment 98 wherein the solvent is DCM.
100. The process of any one of embodiments 90-99 wherein the process reduces formation of des-acyl impurities.
101. The process of any one of embodiments 90-100 wherein the TG Amine or salt thereof is a TG Amine acid salt.
102. The process of any one of embodiments 90-101 wherein the TG Amine acid salt is a phosphate, formate, acetate, trifluoroacetate, or oxalate salt.
103. The process of any one of embodiments 90-102 wherein the TG Amine acid salt is trifluoroacetate or oxalate salt.
104. The process of embodiment 103 wherein the TG Amine acid salt is trifluoroacetate salt.
105. The process of any one of embodiments 90-104 wherein the process results in high purity of the TG PEG product of Step 5b while reducing the amount of a TG Amine acetamide side product.
106. The process of any one of embodiments 90-105 wherein the TG Amine or salt thereof comprises less than about 10%, 9%, 8%, 7%, 6%, 5%, 4%, 3%, or 2% acetamide side product.
107. The process of any one of embodiments 90-106 wherein the TG Amine or salt thereof produced from the improved step 5a process has greater than or equal to about 97%, 98%, or 99% purity as measured by LC.
108. The process of any one of embodiments 90-107 wherein the TG Amine or salt thereof is telescoped into Step 5b without isolation of the TG Amine or salt thereof.
109. The process of any one of embodiments 90-108 wherein the process comprises the reagents in the below scheme:

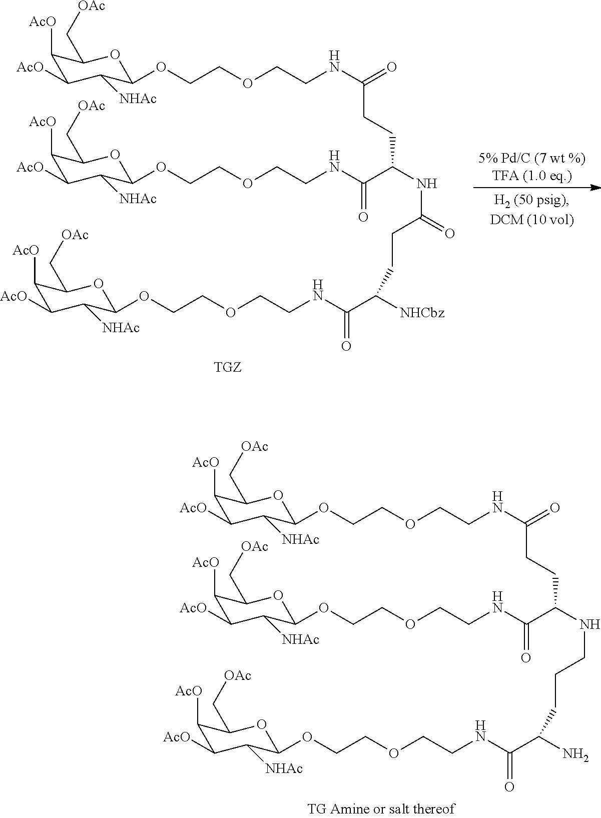 [SMILES: CC(=O)NC1C(OCCOCCNC(=O)CC[C@H](NC(=O)CC[C@H](NC(=O)OCc2ccccc2)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O ; CC(=O)NC1C(OCCOCCNC(=O)CC[C@H](NCCC[C@H](N)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]

110. A process for preparing TG PEG

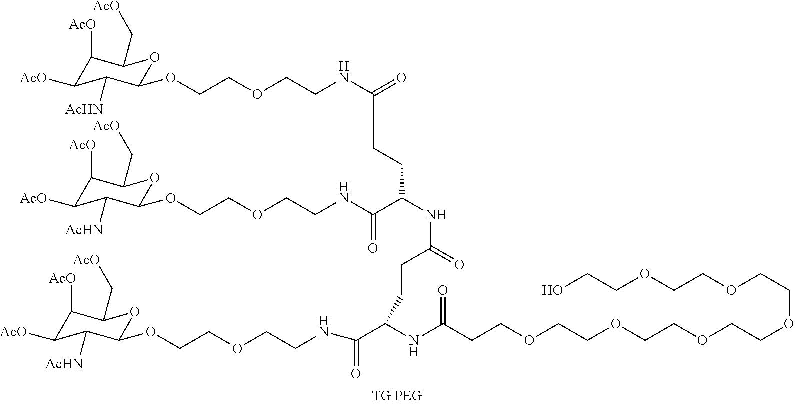

said process comprising treating a solution of TG Amine and PEG acid with TBTU.
111. The process of embodiment 110, wherein the solution further comprises abase.
112. The process of embodiment 111 wherein the base is N,N-Diisopropylethylamine (DIPEA).
113. The process of any one of embodiments 110-112, wherein the TBTU is added over a period of time of about 30 min to about 1.5 hours.
114. The process of any one of embodiments 110-112, wherein the TBTU is added over a period of time of about 1-1.5 hour.
115. The process of any one of embodiments 110-114, wherein the TG PEG is produced in greater than or equal to about 90% purity as measured by LC.
116. The process of any one of embodiments 110-115, wherein TG PEG dimer impurity is present at the end of reaction at an amount of less than 10% as measured by LC.
117. The process of any one of embodiments 110-116 wherein the process comprises the reagents in the below scheme:

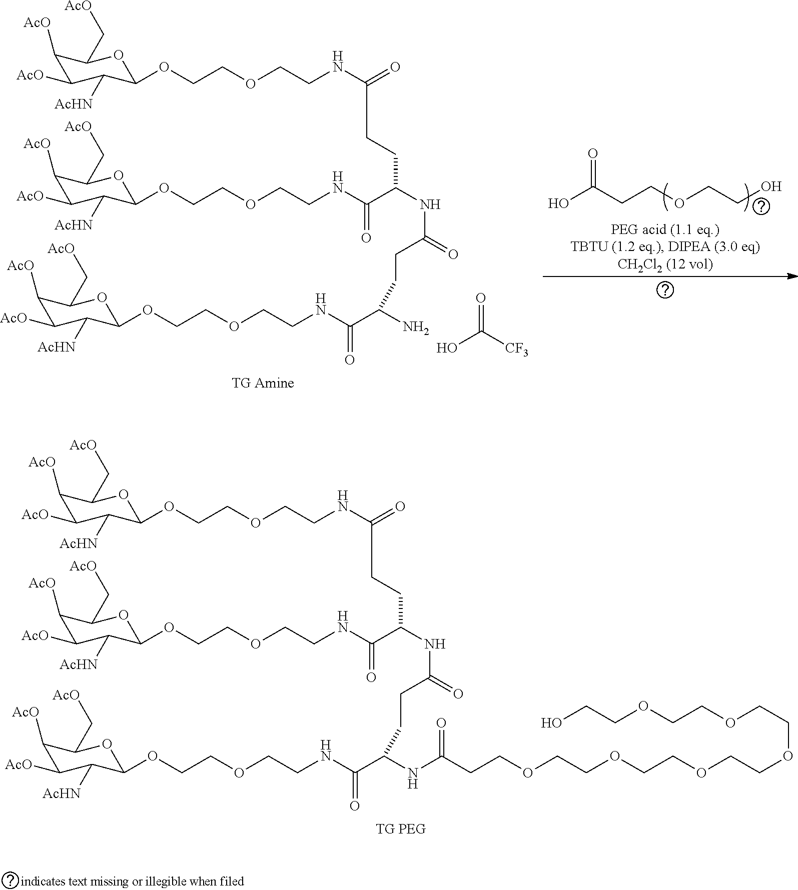

118. A process of preparing NAG-25 comprising treating TG PEG with an activator and a phosphitylating reagent.
119. The process of embodiment 118 wherein the activator is tetrazole, DCI, ETT, or Benzothiotetrazole (BTT).
120. The process of embodiment 118 wherein the activator is tetrazole, DCI, or ETT.
121. The process of embodiment 118 wherein the activator is tetrazole.
122. The process of embodiment 121 wherein the tetrazole is added in about 0.2 eq to 1.2 eq.
123. The process of any one of embodiments 118-122, wherein said process is performed in the presence of a base.
124. The process of any one of embodiment 118-123 wherein the base is NMI.
125. The process of any one of embodiments 118-124 wherein the phosphitylating reagent is 2-cyanoethyl-N, N, N′, N′-tetraisopropylphosphorodiamidite or 2-Cyanoethyl N,N-diisopropylchlorophosphoramidite.
126. The process of any one of embodiment 118-125 further comprising extraction, filtration, precipitation, and drying.
127. The process of embodiment 126, where precipitation comprises precipitation of NAG-25 from DCM/heptane.
128. A process for preparing a NAG-25

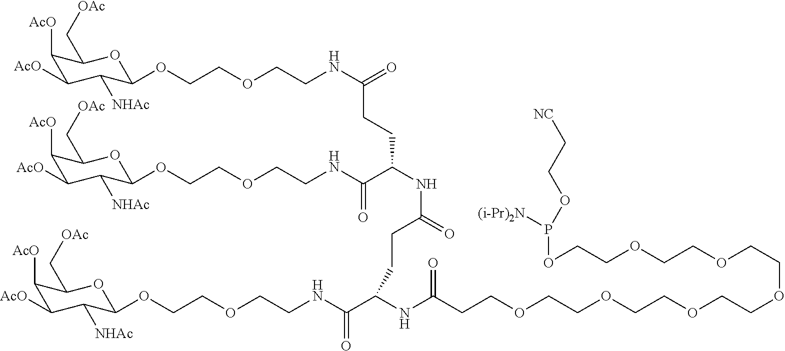

said process comprising any one of the embodiments for steps 1, 2, 3, 4a, 3b, 3a, 4, 5, or 6.
129. The process of embodiment 128, wherein the process comprises preparing t-Butyl Core by the process of step 1 and transforming the t-Butyl Core into NAG-25.
130. The process of embodiment 128, wherein the process comprises preparing Triacid by the process of step 2 and transforming the Triacid into NAG-25.
131. The process of embodiment 128, wherein the process comprises preparing TGZ by the process of step 4b and transforming the TGZ into NAG-25.
132. The process of embodiment 128, wherein the process comprises preparing NAG-Z by the process of step 3b and transforming the NAG-Z into NAG-25.
133. The process of embodiment 128, wherein the process comprises preparing TG Amine by the process of step 5a and transforming the TG Amine into NAG-25.
134. The process of embodiment 128, wherein the process comprises preparing TG PEG by the process of step 5b and transforming the TG PEG into NAG-25.
135. A process for preparing NAG-25:

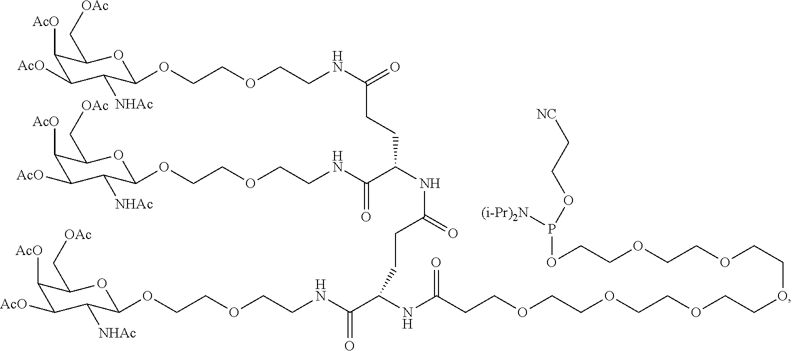

comprising treating a mixture of TG Amine and PEG acid with TBTU to afford TG PEG

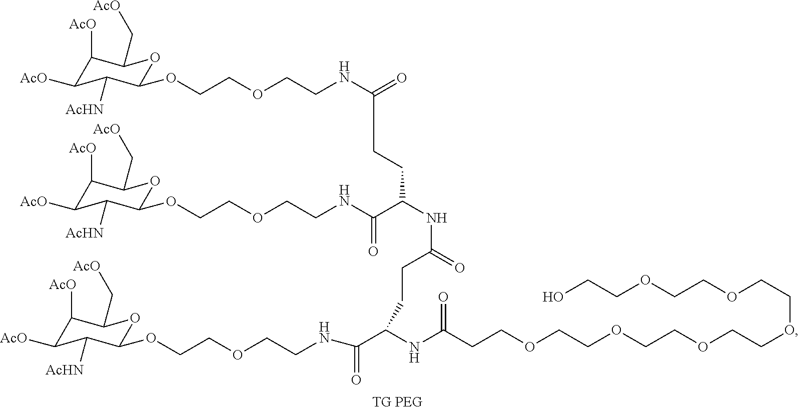

transforming TG PEG into NAG-25.
136. A process for preparing NAG-25, comprising a high pressure hydrogenolysis for a benzyl deprotection of TGZ to form the TG Amine.

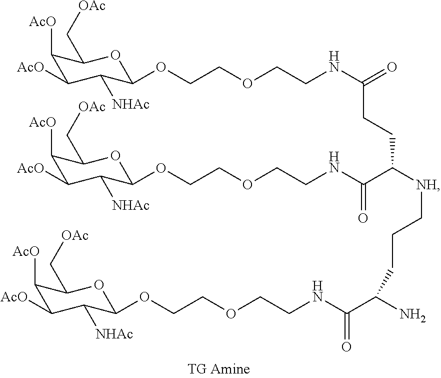 [SMILES: CC(=O)NC1C(OCCOCCNC(=O)CC[C@H](NCCC[C@H](N)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]

or a salt thereof,
and transforming TG Amine, or a salt thereof, into NAG-25.
137. A process for preparing NAG-25, comprising reacting Triacid

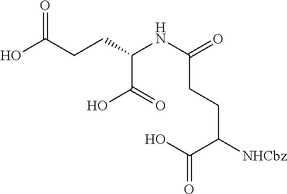 [SMILES: O=C(O)CC[C@H](NC(=O)CCC(NC(=O)OCc1ccccc1)C(=O)O)C(=O)O]

with NAG-H or salt thereof in the presence of a coupling reagent and a base to form TGZ, and transforning TGZ into NAG-25.
138. The process of embodiment 137, wherein TGZ is further precipitated from a solvent/antisolvent system.
139. A process for preparing NAG-25, comprising reacting t-Butyl core with an acid to form the Triacid, and transforming the Triacid into NAG-25.
140. The process of embodiment 139, wherein the Triacid is crystalline.
141. The process of embodiment 140, wherein the Triacid is crystalline Fonn I.
142. A process for preparing NAG-25, comprising reacting GluZ with GluOtBu or salt thereof to form the t-Butyl core, and transforming the t-Butyl Core into NAG-25.
143. A process for preparing a NAG-25, said process comprising a precipitation of TGZ from solvent/antisolvent system.
144. A process for preparing a NAG-25, said process comprising a Triacid intermediate that is crystalline.
145. The process of embodiment 144, wherein the crystalline Triacid is crystalline Form I of Triacid, characterized by an X-ray powder diffractogram having at least a signal at three two-theta values chosen from 7.4±0.2, 9.2±0.2, 21.0±0.2, 14.6±0.2, 18.3±0.2, and 19.3±0.2.
146. The process of embodiment 144, wherein the crystalline Triacid is crystalline Form I of Triacid, characterized by an X-ray powder diffractogram having a signal at two-theta values of 7.4±0.2, 9.2±0.2, and 21.0±0.2.
147. The process of any one of embodiments 144-166, wherein the crystalline Triacid is crystalline Form I of Triacid, characterized by an X-ray powder diffractogram substantially similar to that in FIG. 2.
148. The process of any one of embodiments 144-147, wherein the crystalline Triacid is crystalline Form II of Triacid, characterized by an X-ray powder diffractogram substantially similar to that in FIG. 3.
149. A process for preparing Methyl Core:

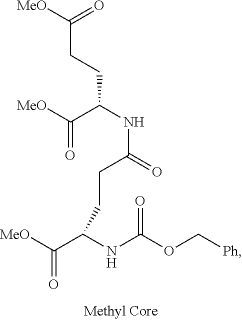 [SMILES: COC(=O)CC[C@H](NC(=O)CC[C@H](NC(=O)OCc1ccccc1)C(=O)OC)C(=O)OC]

said process comprising reacting z-L-Glu-OMe:

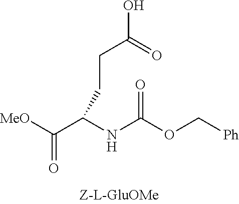 [SMILES: COC(=O)[C@H](CCC(=O)O)NC(=O)OCc1ccccc1]

with L-Glutamic acid dimethyl ester (di(OMe)Glu):

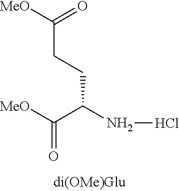 [SMILES: COC(=O)CC[C@H](NCl)C(=O)OC]

to produce Methyl Core.
150. The process of embodiment 149, wherein the reaction is performed in the presence of a base and a coupling reagent.
151. The process of embodiment 150, wherein the base is added to a z-L-GluOMe solution prior to addition of the coupling reagent and di(OMe)Glu to the reaction.
152. The process of embodiment 151, wherein the coupling reagent is added to the solution of z-L-Glu-OMe and base prior to addition of di(OMe)Glu to the reaction.
153. The process of any one of embodiments 149-152, further comprising a solvent.
154. The process of any one of embodiments 150-153, wherein the base is N-Methylmorpholine (NMM) or di-isopropylethylamine.
155. The process of any one of embodiments 150-154, wherein the coupling reagent is isobutyl chloroformate (IBCF), TBTU, HATU, EDC, or DCC.
156. The process of any one of embodiments 153-155, wherein the solvent is THF or MeTHF.
157. The process of any one of embodiments 149-156, wherein the reaction temperature prior to and during the reaction with coupling reagent is about −20° C. to about −10° C.
158. The process of embodiment 157, wherein the reaction temperature is about −18° C. to about −13° C.
159. The process of embodiment 157 or 158, wherein the reaction temperature after addition of di(OMe)Glu is increased to about 5° C. to about 15° C.
160. The process of embodiment 159, wherein the reaction temperature is increased to about 7° C. to about 12° C.
161. The process of any one of embodiments 149-160, further comprising an additional charge of coupling reagent, base, and/or z-L-Glu-OMe.
162. The process of any one of embodiments 149-161, further comprising washing the reaction with aqueous acid followed by washing the reaction with aqueous base.
163. The process of any one of embodiments 149-162, further comprising crystallization by adding anti-solvent.
164. The process of any one of embodiments 149-163, wherein the process comprises the reagents and conditions in the below scheme:

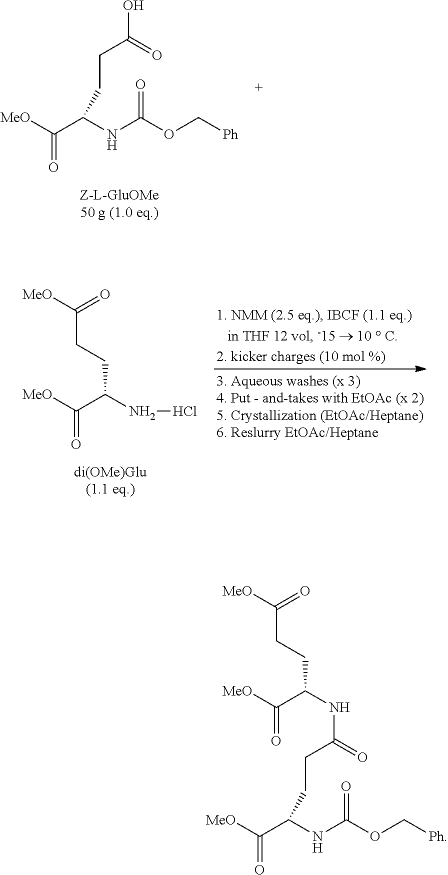 [SMILES: COC(=O)[C@H](CCC(=O)O)NC(=O)OCc1ccccc1 ; COC(=O)CC[C@H](NCl)C(=O)OC ; COC(=O)CC[C@H](NC(=O)CC[C@H](NC(=O)OCc1ccccc1)C(=O)OC)C(=O)OC]

165. A process for preparing Triol:

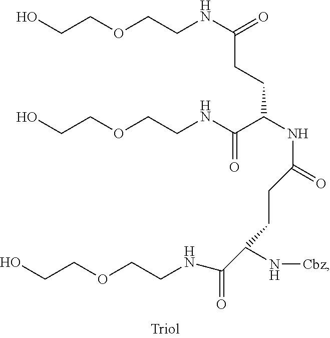

the process comprising reacting Methyl Core with 2-(2-aminoethoxy)ethanol:

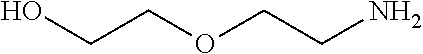

to produce Triol.
166. The process of embodiment 165, wherein the process is performed with neat 2-(2-aminoethoxy)ethanol.
167. The process of embodiment 165 or 166, wherein the reaction temperature is at about 25° C. to 35° C.
168. The process of embodiment 167, wherein the reaction temperature is about 30° C.
169. The process of any one of embodiments 165-168, further comprising adding an anti-solvent to result in precipitation of the Triol.
170. The process of embodiment 169, wherein the anti-solvent is ethyl acetate, methyl-tert-butyl ether, or iso-propylacetate.
171. The process of any one of embodiments 165-170, further comprising adding a Triol seed crystal.
172. The process of any one of embodiments 165-171, wherein the process comprises the reagents and conditions in the below scheme:

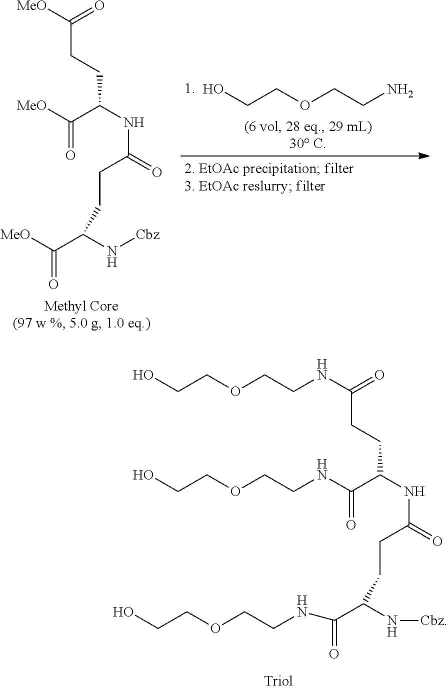

173. A process for preparing TGZ, the process comprising reacting Triol:

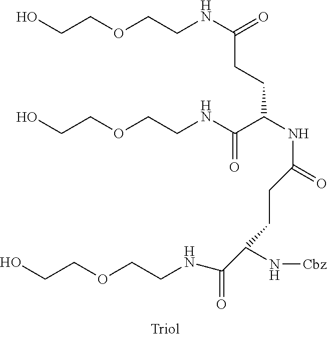

with Beta-D-Galactosamine pentaacetate:

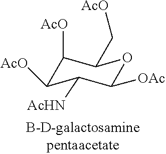 [SMILES: CC(=O)NC1C(OC(C)=O)OC(COC(C)=O)C(OC(C)=O)C1OC(C)=O]

pentaacetate to produce TGZ.
174. The process of embodiment 173, wherein Beta-D-Galactosamine pentaacetate is first reacted with a silyl-triflate to produce an oxazoline solution.
175. The process of embodiment 174, wherein the silyl triflate is Trimethylsilyl trifluoromethanesulfonate (TMSOTf) or Triisopropylsilyl Trifluoromethanesulfonate (TIPSOTf).
176. The process of embodiment 175, further comprising solvent.
177. The process of embodiment 176, wherein the solvent is dichloroethane (DCE) or dichloromethane (DCM).
178. The process of any one of embodiments 173-177, wherein the reaction temperature is about 35° C. to about 45° C.
179. The process of embodiment 178, wherein the reaction temperature is about 40° C.
180. The process of embodiment 178 or 179, wherein the reaction temperature is decreased to about 20° C. to about 28° C.
181. The process of embodiment 180, wherein the reaction temperature is about 23° C.
182. The process of embodiment 173, wherein the Triol is mixed with sodium bicarbonate (NaHCO₃) to produce a slurry mixture of Triol and NaHCO₃ prior to addition of Beta-D-Galactosamine pentaacetate or its oxazoline solution.
183. The process of embodiment 182, wherein the slurry mixture of Triol and NaHCO₃ further comprises a solvent.
184. The process of embodiment 183, wherein the solvent is dichloromethane (DCM), dichloroethane, or acetonitrile.
185. The process of any one of embodiments 173-184, wherein the oxazoline solution is added to the slurry mixture of triol and NaHCO₃. 186. The process of embodiment 173 or 185, wherein the reaction temperature is about 20° C. to about 30° C.
187. The process of embodiment 186, wherein the reaction temperature is about 25° C.
188. The process of any one of embodiments 173-187, wherein TGZ is further precipitated from a solvent/antisolvent system.
189. The process of any one of embodiments 173-188, wherein an anti-solvent is added to a solution comprising TGZ and the solvent.
190. The process of any one of embodiments 188 or 189, wherein the anti-solvent is dimethoxyethane.
191. The process of any one of embodiments 173-190, further comprising any one of embodiments 129-144 or 145-152 or embodiments 149-164 or 165-172.
192. The process of any one of embodiments 173-191, wherein the process comprises the reagents and conditions in the below scheme:

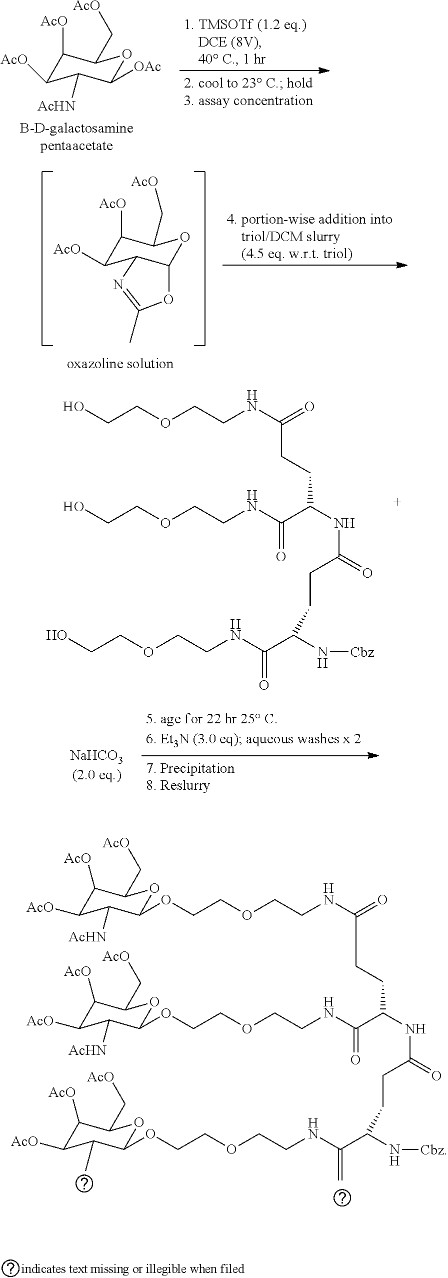

193. A process for preparing a NAG-25, said process comprising any one of embodiments 149-164 for step A-1, any one of embodiments 165-172 for step A-2, any one of embodiments 173-192 for step A-3, or any one of embodiments 71-89 for step 3b.
194. The process of embodiment 193 further comprising any one of embodiments 90-109 for step 5a, embodiments 110-117 for step 5b, or embodiments 118-127 for step 6.
195. A process for preparing a NAG-25, said process comprising reacting z-L-Glu-OMe and L-Glutamic acid dimethyl ester to form the Methyl Core, and transforming the Methyl Core into NAG-25.
196. A process for preparing a NAG-25, said process comprising reacting Methyl Core with 2-(2-aminoethoxy)ethanol to form the Triol, and transforming the Triol into NAG-25.
197. A process for preparing a NAG-25, said process comprising reacting Triol with Beta-D-Galactosamine pentaacetate to form TGZ, and transforming the TGZ into NAG-25.
198. The process of embodiment 197, wherein TGZ is further precipitated from a solvent/antisolvent system.
199. A process for preparing a NAG-25, said process comprising a Triol intermediate.
200. A compound

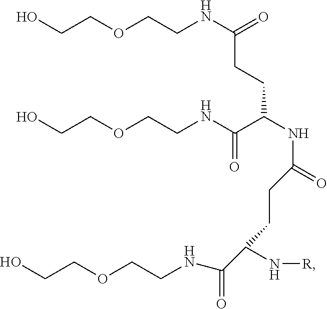

wherein R is H or a Cbz protecting group.

It is to be understood that while the disclosure is read in conjunction with the detailed description thereof, the foregoing description is intended to illustrate and not limit the scope of the disclosure, which is defined by the scope of the appended claims. Other aspects, advantages, and modifications are within the scope of the following claims. For example, as shown in the following Examples, which are provided for illustrative purposes only and are not to be construed as limiting the scope of the appended claims.

### EXAMPLES

### Example 1: Step 1

An example of Step 1 is shown below.

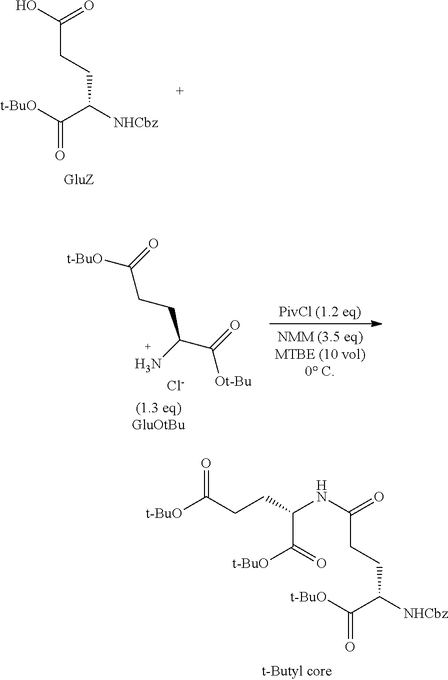

A GluZ solution is prepared as follows: 1.0 eq of GluZ, 3.5 eq of NMM, and 5 vol of MTBE is charged into a clean inert reactor 1. Into a separate clean inert reactor 2, 1.2 eq of PivCl and 5 vol of MTBE are charged at 0° C. Using inverse addition, the GluZ solution in reactor 1 is added to reactor 2 over 1 hour. A white slurry is formed and stirred for at least 30 mins. 1.3 eq of GluOtBu hydrochloride is then charged portion wise every 15 min (exothermic). Afterwards the slurry is held for at least 30 min at 0° C. If conversion is <97% then charge 0.5 eq of NMM, 0.4 eq of PivCl, and 0.45 eq of GluOtBu HCl. The reaction mixture can be held for 24 h at 0° C. if needed. 12.5 vol of 0.5N HCl is slowly added to the reaction mixture (exothermic) at 0° C. and agitated for 30 min. The phases are left to settle and then are separated. The organic layer is washed with 10 vol of 1N sodium carbonate. The phases are left to settle and are then separated. The organic layer is washed with water (10 vol). The phases are left to settle and then are separated. The organic layer can be held at 0° C. overnight if needed. The organic layer is concentrated down to 6 vol at 35° C., and heptane (10 vol) is charged all at once at 35° C. Jacket temperature (Tj) of the reactor is set to 50° C. and agitated until fully dissolved. Tj is then set to 40° C. and 0.5 wt % seed is charged. A t-Butyl core that may be used as a seed can be prepared as described in U.S. Pat. No. 10,246,709 or by methods known to one of ordinary skill in the art. The slurry is agitated for at least 30 min. Then, 15 vol of heptane is charged over 3 hours at 40° C. Once heptane addition is complete, Tj is slowly cooled to 20° C. and held overnight. The slurry is stirred at 20° C. overnight. The slurry is filtered, and the filter cake is washed twice with Heptane (3 vol). The solids of t-Butyl core are dried under nitrogen at 20° C. overnight.

¹H NMR (500 MHz, DMSO-d6) δ ppm 1.40 (d, J=1.30 Hz, 27H) 1.72-1.85 (m, 2H) 1.89-2.02 (m, 2H) 2.21-2.32 (m, 4H) 4.14-4.19 (m, 1H) 4.97-5.13 (m, 2H) 7.29-7.43 (m, 8H) 7.48 (d, J=7.27 Hz, 2H) 7.53 (t, J=7.60 Hz, 2H) 7.59-7.68 (m, 2H) 8.01 (d, J=7.59 Hz, 2H)

Improved reaction conversion also resulted when paired with 2.4 eq of NMM in place of 3.5 eq.

### PivCl and GluOtBu HCl Screen

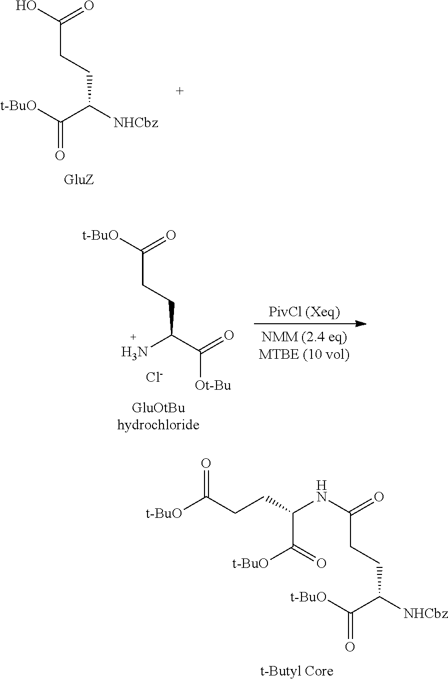

An equivalents screen was conducted for PivCl and GluOtBu HCl to further increase the reaction conversion and yield. The conversion increased with higher equivalents of PivCl and GluOtBu HCl. However, the levels of the impurities increased as well. The goal was to find the amount of PivCl that resulted in increased conversion to t-Butyl Core while not producing higher levels of impurities. The highest conversion was achieved with 1.2 eq of PivCl and 1.3 eq GluOtBu HCl.

The table below provides the LCAP of t-Butyl Core from PivCl equivalent screen

| | | | LCAP | LCAP | | |
|---|---|---|---|---|---|---|
| | PivCl | GluOtBu | starting | t-butyl | conversion | LCAP |
| Reaction | eq | HCl eq | material | core | to product | Impurities |
| 1 | 1.1 | 1.1 | 7.3 | 91.2 | 92.8 | 1.6 |
| 2 | 1.1 | 1.2 | 6.4 | 91.7 | 93.6 | 1.9 |
| 3 | 1.2 | 1.2 | 4.8 | 91.4 | 95.2 | 3.9 |
| 4 | 1.2 | 1.3 | 4.5 | 92.7 | 95.5 | 2.8 |
| 5 | 1.3 | 1.4 | 5.7 | 88.4 | 94.3 | 5.9 |

### Base Equivalent Screen

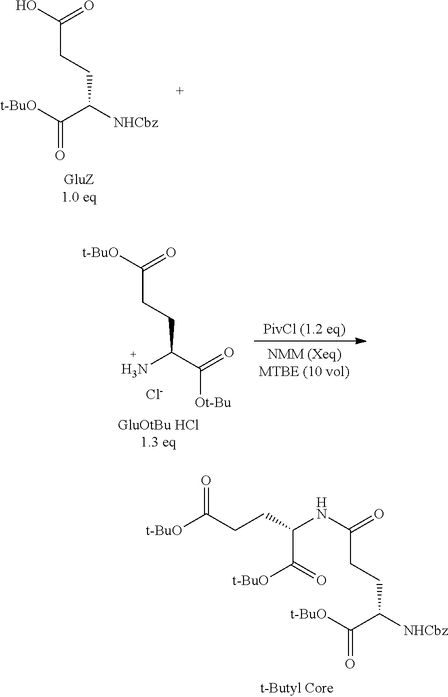

Using 1.2 eq PivCl and 1.3 eq GluOtBu HCl, the equivalents of NMM were examined. Experiments resulted in unreacted mixed anhydride still present at the end of reaction. Determination of the amount of NMM would free base the additional GluOtBu HCl and allow the reaction to progress. Having excess NMM (3-3.5 eq) resulted in no residual mixed anhydride. Higher equivalents (4-5 eq of NMM) were investigated, but did not result in further reaction improvement.

The table below shows LCAP of t-butyl core for NMM equivalent screen

| NMM | Mix | GluZ | t-butyl core |
|---|---|---|---|
| (eq) | anhydride | (LCAP) | (LCAP) |
| 3.0 | N.D. | 3.7 | 96.3 |
| 3.5 | N.D. | 2.2 | 97.1 |

### Comparison of Normal Addition vs Inverse Addition

Since it was hypothesized that normal addition had the potential for symmetrical anhydride formation, another study comparing normal vs inverse addition was performed. A comparison between both normal addition and inverse addition was conducted using GluOtBu (1.3 eq), NMM (3.5 eq), PivCl (1.2 eq). Under normal addition, a solution of PivCl in MTBE was added to the solution of GluZ, NMM, MTBE and the reaction was cooled to 0° C. and held for 30 minutes. Afterwards, GluOtBu hydrochloride was added in three portions every 15 minutes and then the reaction was held for 30 minutes and sampled for liquid chromatography (LC) measurements at various time points.

The table below shows a comparison of inverse and normal addition for Step 1 using the conditions shown below:

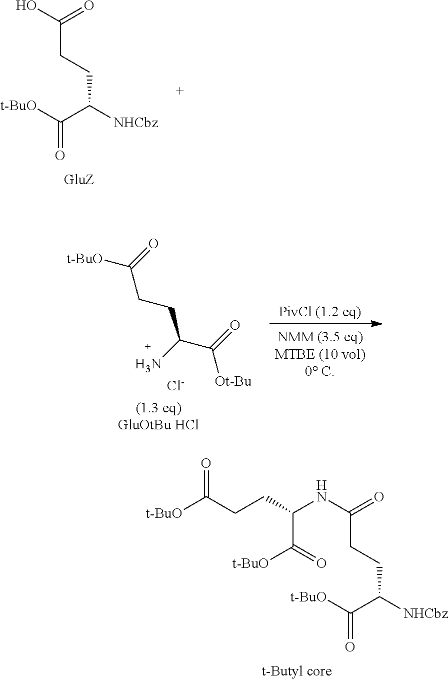

The table below shows that normal addition resulted in about 88.7% conversion of t-butyl core while the inverse addition demonstrated about 97.5% or 96.1% conversion.

| Method of addition | LCAP of GluZZ | LCAP of t-butyl core |
|---|---|---|
| Normal addition | 11.3 | 88.7% |
| Inverse addition | 2.5 | 97.5 |
| Inverse addition | 4.0 | 96.1 |

### Screen for Solvent/Antisolvent for Crystallization

A High Throughput Experimentation (HTE) solubility screen was conducted to determine the solvent/anti-solvent combination for the crystallization and align the reaction solvent with the crystallization solvent. Solubility studies of t-Butyl Core at 20° C. were conducted for various percentages of heptane. For 2-MeTHF, MIBK, and IPAc, the t-Butyl Core was found to have a steep solubility cliff, going from 50 mg/mL to 10 mg/mL between 80 to 90% heptane, which would not be optimal in designing a crystallization. The solubility screen pointed to MTBE/heptane being one example of a useful solvent mixture for crystallization because of its gradual solubility curve.

### Example 2: Step 2

An example of Step 2 is shown below.

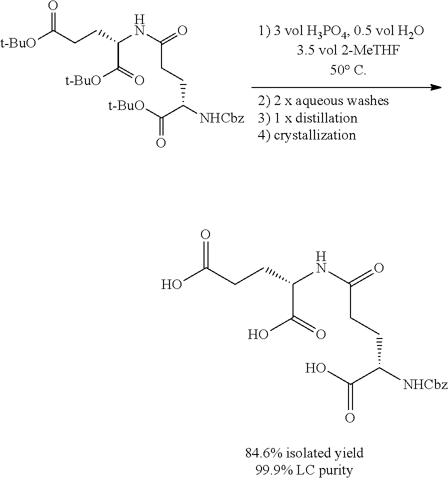 [SMILES: CC(C)(C)OC(=O)CC[C@H](NC(=O)CC[C@H](NC(=O)OCc1ccccc1)C(=O)OC(C)(C)C)C(=O)OC(C)(C)C ; O=C(O)CC[C@H](NC(=O)CC[C@H](NC(=O)OCc1ccccc1)C(=O)O)C(=O)O]

3 vol H₃PO₄ is added to a mixture of t-Butyl core (1 eq), 2-MeTHF (3.5 vol) and 0.5 vol water, and the mixture is heated to 50° C. for 4.5 hours. The phosphoric acid is effectively removed by aqueous extraction: the reaction mixture is diluted with isopropylacetate (iPAC) (10 vol) and washed with 20 wt % ammonium sulfate ((NH₄)₂SO₄) aqueous solution (5 vol). Then, the organic layer is washed with 1.5 vol water. Following an azeotropic distillation, acetone (4 vol) is charged and the mixture is polish filtered to remove residual salts. Acetone (2 vol) is rinsed through the filter into the reactor. The mixture is heated to 45° C. and toluene (6 vol) is charged to the reactor. 1 wt % Triacid seed is charged at 45° C. The slurry is agitated at 45° C. After holding for 6-18 h, toluene (6 vol) is charged over 2.5 hours and the solution is stirred until <6 mg/ml mother liquor concentration is reached. The reactor is cooled to 20° C. and stirred for no less than 0.5 hr. The solid product is filtered and washed once with pre-mixed 2:1 toluene:acetone (3 vol). The cake is vacuum dried under a stream of nitrogen at 20° C. For Triacid that was prepared in a similar procedure as described above, mass spectrometry from UPLC-MS is m/z (ESI, positive ion): 411.27 (M+H)⁺. The Triacid isolated from this preparation is crystalline Form I.

As an alternative to using IPAc, aqueous extraction was performed with MeTHF by washing the crude reaction mixture with 20 wt % ammonium sulfate (5 vol) and water (2 vol). After MeTHF (6-10 vol) was added, the mixture was distilled, and the MeTHF addition with distillation was repeated up to two more times. The next steps continued with acetone and polish filtering as described previously.

While investigating the deprotection step, it was found that adding water as a tert-butyl acceptor improved the rate and conversion of the deprotection reaction (Table 1) compared with MeTHF as the only solvent (Table 1; conditions=2 vol H₃PO₄, 60° C.). Additional experiments found that 3 vol H₃PO₄ with 0.5 vol water at 55 or 50° C. provided triacid that has 94-95% purity by liquid chromatography (LC) and with less impurities than the 2 vol H₃PO₄/60° C. system.

TABLE 1
Effect of water upon deprotection profile.

| | | 5 vol solvent, 2 vol H₃PO₄, 60 C. | | | | |
|---|---|---|---|---|---|---|
| | | | LCAP | | | |
| | | | tBu | LCAP | LCAP | LCAP |
| Reaction | Solvent | Time/h | core | monoacid | diacid | triacid |
| 1 | MeTHF 5 V | 5 | 0.66 | 7.27 | 32.92 | 54.46 |
| 2 | MeTHF 3.5 V + | 5 | 0 | 0 | 1.92 | 92.43 |
| | water 1.5 V | | | | | |

### Preparation of Triacid Crystalline Form I and XRPD Characterization

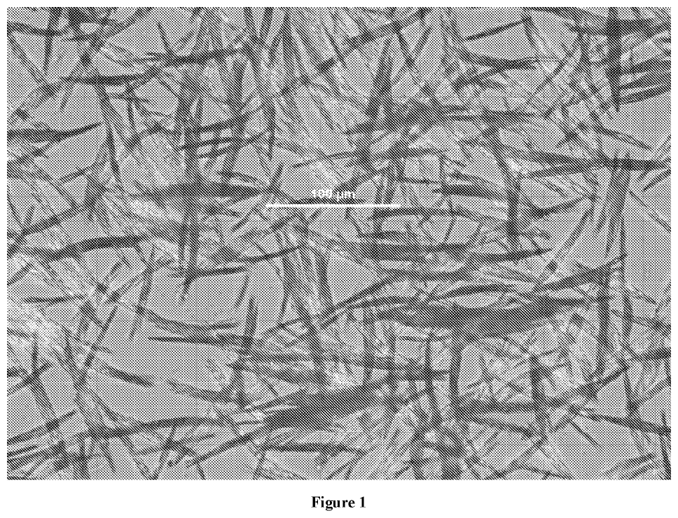

During process development, acetone/toluene was discovered as a crystallization system via high throughput screening. In additional experiments, Triacid crystalline Form I was generated by seeding a 49:51 toluene:acetone mixture at 45° C. and allowing desaturation at 45° C. resulted in formation of crystalline triacid. FIG. 1 is an example of crystalline Triacid Form I. The resulting solids are typically formed in >99% purity by LC.

Crystal Preparation: Triacid crystalline form I was formed by charging acetone (4.9 vol) to a distilled crude reaction stream (following extractive work-up, distillation and two put-takes with acetone). The mixture was heated to 48° C. and 4.7 vol toluene was charged. At 45° C., seed was charged. Agitation was continued for ˜20 h at 45° C., before charging 3.5 vol further toluene. The reactor was cooled to 20° C. over 1 h, the solids filtered and washed once with 3:2 toluene:acetone, then dried under vacuum.

X-Ray Powder Diffraction: X-ray powder diffraction data were obtained on a PANalytical X′Pert PRO X-ray diffraction system with RTMS detector. Samples were scanned in continuous mode from 5-45° (2θ) with step size of 0.0334° at 45 kV and 40 mA with CuKα radiation (1.54 Å). The incident beam path was equipped with a 0.02 rad soller slit, 15 mm mask, 4° fixed anti-scatter slit and a programmable divergence slit. The diffracted beam was equipped with a 0.02 rad soller slit, programmable anti-scatter slit and a 0.02 mm nickel filter. Samples were prepared on a low background sample holder and placed on a spinning stage with a rotation time of 2 s. The XRPD pattern of the toluene/acetone form of Triacid crystalline Form I is shown in FIG. 2 and the XRPD peaks are listed in Table 2 below.

TABLE 2
XRPD signals for Triacid crystal Form I

| Pos. [°2Th.] | d-spacing [Å] |
|---|---|
| 7.42 | 11.92 |
| 9.21 | 9.60 |
| 10.20 | 8.68 |
| 14.56 | 6.08 |
| 18.34 | 4.84 |
| 19.30 | 4.60 |
| 20.97 | 4.24 |
| 22.95 | 3.88 |

### Preparation of Triacid Crystal Form II

2 g of Triacid was charged to a 40 mL vial with stirrer bar. 1 V (2 mL) 2-MeTHF and 1.5 V (3 mL) iPAc were added. The mixture was heated to 50° C. with stirring to dissolve the triacid. 13.5 V iPAc (27 mL) was added to the mixture. On addition of iPAc, the solution clouded and some oil was observed. The temperature was increased until an internal temperature of 50° C. was reached. On heating and stirring, the oil turns into loose solid. The slurry was held for 15 mins at 50° C. A sample of the mother liquor concentration measured 58 mg/ml triacid. The mixture was cooled to 45° C., resulting in a very thick slurry, and then cooled to room temperature. The concentration of the mother liquor was measured as 5 mg/mL triacid. At this point, the slurry was transferred to a filter. 10 V iPAc (20 mL) were charged to loosen solid remaining in the vial and the mixture was also charged to the filter. Finally, the vial was rinsed with 2.5 V iPAc (5 mL) and the mixture added to the filter. The solids were dried under vacuum with nitrogen flow overnight. 1.55 g solid was isolated. Quantitative NMR (using a maleic acid internal standard, d6-DMSO solvent) indicated the material was 86 wt % triacid and 11 wt % iPAc. The XRPD pattern of the Triacid crystalline Form II is shown in FIG. 3. This resulting Triacid crystal Form II can be used as a Triacid seed in a series of crystallizations using the Step 2 example described above to produce Triacid crystal Form I.

### Example 3: Step 3a

Step 3a: An example of Step 3a is shown below.

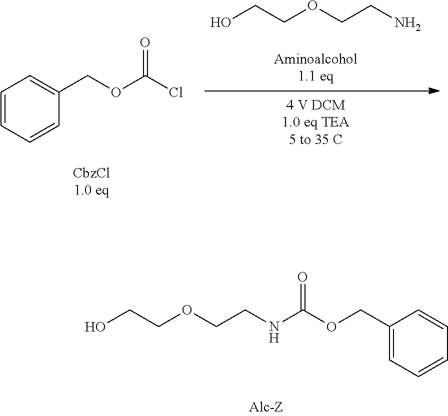 [SMILES: NCCOCCO ; O=C(Cl)OCc1ccccc1 ; O=C(NCCOCCO)OCc1ccccc1]

A solution of aminoalcohol (1.1 eq), TEA (1.0 eq), and DCM (4 vol) is cooled to 5° C. CbzCl (1.0 eq) is added over an hour, and once addition is complete, the solution is heated to 35° C. for 12 hours.

The solution is cooled to 20° C. and quenched by addition of water (2 vol). The aqueous layer is collected and back extracted with DCM (2 vol). The combined organic layers are washed two times with 10% NaCl (2 vol), and solvent swapped into ACN (5 vol). The Alcohol-Z (abbreviation: Alc-Z) product is then isolated as a clear oil or stored in ACN (5 vol) for use in the next step. ¹H NMR (400 MHz, CHLOROFORM-d) δ 3.41 (q, J=5.11 Hz, 2H) 3.53-3.60 (m, 4H) 3.69-3.76 (m, 2H), 5.11 (s, 2H), 5.35 (br s, 1H) 7.29-7.43 (m, 5H).

### Example 4: Step 3b

Step 3b: An example of Step 3b is shown below.

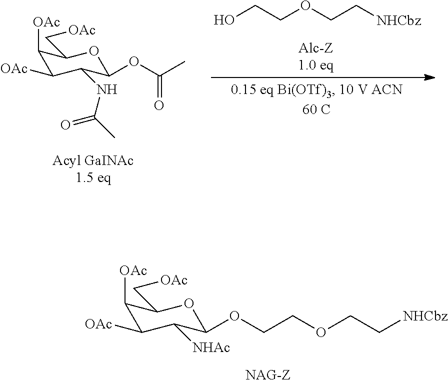 [SMILES: O=C(NCCOCCO)OCc1ccccc1 ; CC(=O)NC1C(OC(C)=O)OC(COC(C)=O)C(OC(C)=O)C1OC(C)=O ; CC(=O)NC1C(OCCOCCNC(=O)OCc2ccccc2)OC(COC(C)=O)C(OC(C)=O)C1OC(C)=O]

To an inert flask is added Acyl GalNAc (1.5 eq), Bi(OTf)₃ (0.15 eq), and ACN (5 vol). A solution of Alcohol-Z (1.0 eq) is azeotropically dried and added to the flask in ACN (5 vol). The solution is heated to 60° C. and stirred for 18 hours. After cooling to 20° C., charcoal (50 weight %) is added and stirred at room temperature for 12 hours. The charcoal is then filtered off and water (18.6 vol) is charged to the filtrate. The resulting solution is seeded with NAG-Z (0.5 wt %) and stirred for 30 minutes. If the seed does not hold, the seeding process is repeated until it does. If the seed does hold, water (38 vol) is added over an hour, and the resulting slurry is stirred for 12 hours. The resulting product is filtered, washed with two water washes (2 vol), and vacuum dried under a stream of nitrogen. The NAG-Z product is then obtained as a white solid (71.27%, potency adjusted). ¹H NMR (600 MHz, DMSO-d6) δ 7.78 (d, J=9.20 Hz, 1H), 7.35-7.37 (m, 2H), 7.32-7.35 (m, 2H), 7.30-7.32 (m, 1H), 7.24 (br t, J=5.60 Hz, 1H), 5.21 (d, J=3.40 Hz, 1H), 5.01 (s, 2H), 4.99 (dd, J=3.40, 11.20 Hz, 1H), 4.56 (d, J=8.40 Hz, 1H), 3.99-4.04 (m, 1H), 3.98-4.07 (m, 3H), 3.88 (ddd, J=8.40, 9.20, 11.10 Hz, 1H), 3.74-3.81 (m, 1H), 3.55-3.63 (m, 1H), 3.45-3.55 (m, 2H), 3.38-3.43 (m, 2H), 3.14 (q, J=5.70 Hz, 2H), 2.10 (s, 3H), 1.99 (s, 3H). 1.89 (s, 3H), 1.77 (s, 3H).

Lewis acid screen for Step 3b: Certain Lewis acids commonly used for glycosylation reactions were low yielding for the NAG-Z process. A Lewis acid screen was conducted under conditions described below:

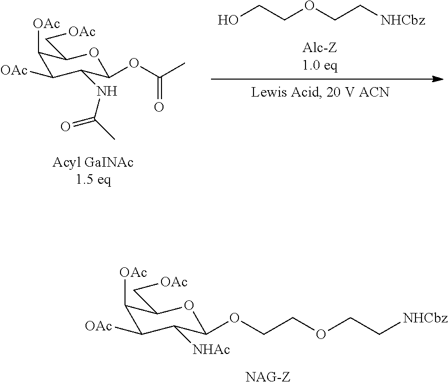 [SMILES: O=C(NCCOCCO)OCc1ccccc1 ; CC(=O)NC1C(OC(C)=O)OC(COC(C)=O)C(OC(C)=O)C1OC(C)=O ; CC(=O)NC1C(OCCOCCNC(=O)OCc2ccccc2)OC(COC(C)=O)C(OC(C)=O)C1OC(C)=O]

It was found that Lewis acids of medium strength were useful for NAG-Z formation. Some Lewis acids were too weak to achieve complete conversion of the Alcohol-Z, while others were too strong and decomposed the resulting NAG-Z into acylated and des-acylated products. The results of two relevant experiments are summarized in the table below. Column A:NAG-Z LCAP shows the LCAP of NAG-Z obtained after 24 hours at the given reaction conditions, and column B:NAG-Z degradation shows the degradation seen after subjecting NAG-Z to the corresponding Lewis acid for 24 hours.

| | | A: NAG-Z | B: NAG-Z |
|---|---|---|---|
| Lewis Acid | Conditions | LCAP | degradation |
| BiCl₃ | 0.15 eq, 60° C. | 11.37 | — |
| Fe(OTf)₂ | 0.15 eq, 60° C. | 17.54 | — |
| In(OTf)₃ | 0.15 eq, 60° C. | 66.66 | — |
| Cu(OTf)₂ | 0.15 eq, 60° C. | 74.91 | — |
| Bi(OTf)₃ | 0.15 eq, 60° C. | 78.85 | 1.79% |
| BF₃OEt₂ | 1 eq, room temperature | 46.57 | 5.43% |
| TBSOTf | 1 eq, room temperature | 66.98 | 8.75% |
| TIPSOTf | 1 eq, room temperature | 62.08 | 25.83% |
| TMSOTf | 1.1 eq, room temperature | 55.02 | 64.03% |

Acyl GalNAc equivalents screen for Step 3b: The equivalents of Acyl GalNAc were tested due to an underlying hydrolysis mechanism shown below.

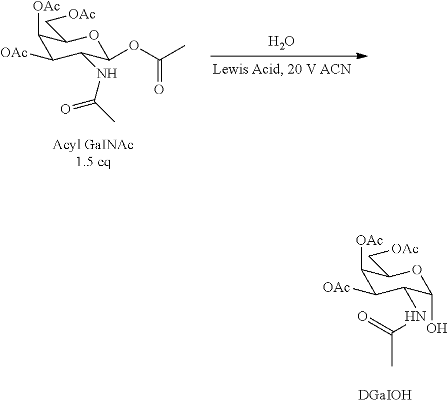 [SMILES: CC(=O)NC1C(OC(C)=O)OC(COC(C)=O)C(OC(C)=O)C1OC(C)=O ; O ; CC(=O)NC1C(O)OC(COC(C)=O)C(OC(C)=O)C1OC(C)=O ; "DGaIOH"]

Since the hydrolysis was difficult to avoid through drying due to the hydroscopic nature of the bismuth catalyst, Alcohol-Z was used as a limiting reagent to improve conversion and the hydrolysis impurity DGalOH could be purged during the NAG-Z crystallization. The conditions used for the equivalents screen are shown below (DGalNAc is same as Acyl GalNAc).

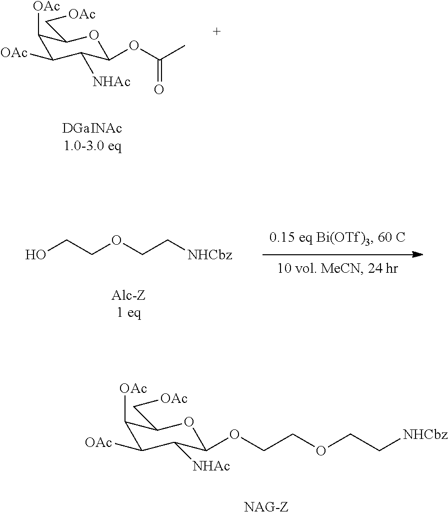 [SMILES: CC(=O)NC1C(OC(C)=O)OC(COC(C)=O)C(OC(C)=O)C1OC(C)=O ; O=C(NCCOCCO)OCc1ccccc1 ; CC(=O)NC1C(OCCOCCNC(=O)OCc2ccccc2)OC(COC(C)=O)C(OC(C)=O)C1OC(C)=O]

The results of the equivalents screen, and major impurities that were found (Acyl GalNAc, DGalOH, Alcohol-Z), are summarized below.

| Acyl GalNAc | DGalOH | Acyl GalNAc | Alcohol-Z | NAG-Z |
|---|---|---|---|---|
| eq | LCAP | LCAP | LCAP | LCAP |
| 1.0 | 12.58 | 1.01 | 20.34 | 49.38 |
| 1.1 | 9.4 | 0.1 | 11.1 | 67.5 |
| 1.2 | 11.76 | 0.27 | 5.48 | 68.94 |
| 1.3 | 13.03 | 2.42 | 3.84 | 63.30 |
| 1.4 | 12.46 | 2.70 | 4.08 | 60.73 |
| 1.5 | 6.53 | 3.18 | 2.32 | 67.87 |
| 2.0 | 15.2 | 20.29 | 0.10 | 60.77 |
| 3.0 | 11.18 | 35.18 | 1.2 | 49.0 |

Solvent volumes for Step 3b: Lower volumes of solvent generally lead to reduced volumes (better from green chemistry/sustainability perspective) and generally leads to lower losses of product to mother liquor. Therefore, several reaction volumes of acetonitrile were screened. The table below summarizes the LCAP for Alcohol-Z, NAG-Z, and Ac-Z impurity:

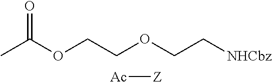

| ACN volumes | Alcohol-Z LCAP | Ac-Z LCAP | NAG-Z LCAP |
|---|---|---|---|
| 3.0 | 3.13 | 3.04 | 73.22 |
| 6.0 | 3.10 | 1.10 | 69.95 |
| 7.5 | 2.50 | 2.56 | 61.72 |
| 8.0 | 2.51 | 1.17 | 60.88 |
| 8.5 | 2.92 | 2.31 | 60.64 |
| 9.0 | 2.60 | 0.38 | 63.29 |
| 10.0 | 2.32 | 0.50 | 67.87 |
| 20.0 | 43.89 | 0 | 42.33 |

### Example 5: Step 4a

Step 4a: An example of Step 4a is shown below.

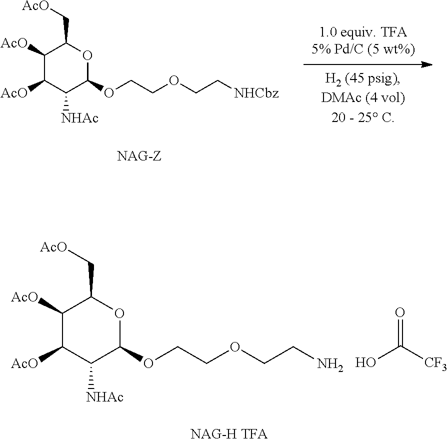 [SMILES: CC(=O)N[C@H]1[C@H](OCCOCCNC(=O)OCc2ccccc2)O[C@H](COC(C)=O)[C@H](OC(C)=O)[C@@H]1OC(C)=O ; CC(=O)N[C@H]1[C@H](OCCOCCN)O[C@H](COC(C)=O)[C@H](OC(C)=O)[C@@H]1OC(C)=O ; O=C(O)C(F)(F)F]

To a 5-L pressure vessel was charged NAG-Z (356.0 g; 97.5 wt %; 610.4 mmol) followed by Pd/C (18.5 g; 5 wt %), DMAc (1.4 L; 4V) and lastly TFA (47 mL; 610.4 mmol). The reactor was sealed and purged with nitrogen gas. The headspace was then purged with hydrogen gas and set to 46 psi (3.1 bar) hydrogen. The reaction was allowed to stir at 21.6 C for 19 h under these conditions. The catalyst was removed via filtration and washed with DMAc (350 ml; IV). The resulting clear solution was assayed to contain 256.9 g NAG-H (96.7% yield) at 97.1 LCAP.

### Example 6: Step 4b

Step 4b: An example of Step 4b is shown below.

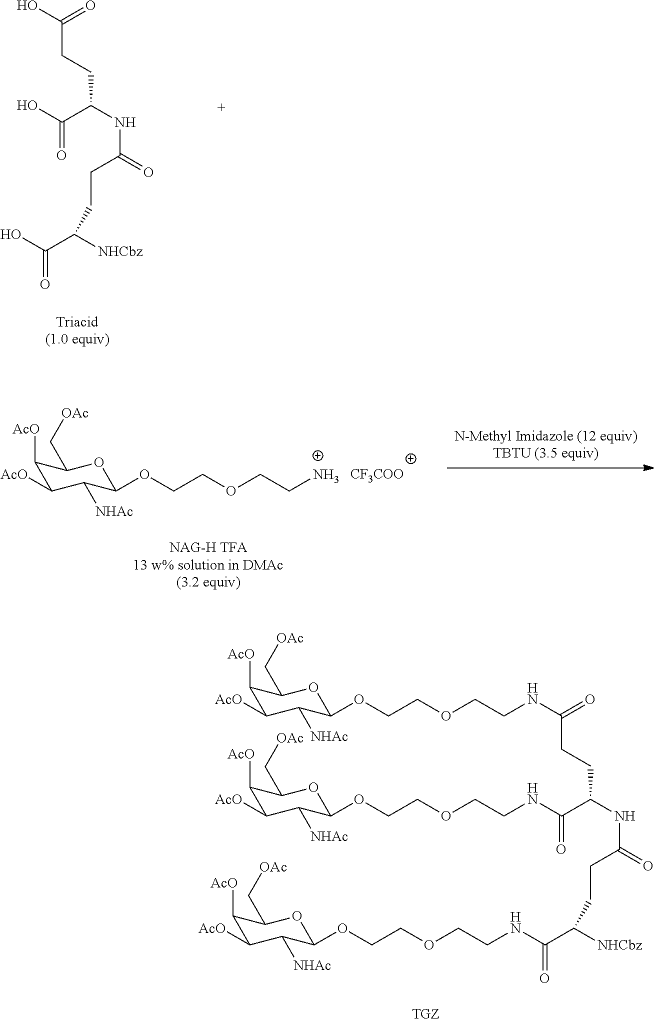

Note: all equivalents/volumes relative to Triacid

To a 5 L jacketed reactor equipped with nitrogen inlet and temperature probe was charged Triacid (55.0 g, 1.0 equiv) and NAG-H TFA solution (13.27 wt % in DMAc, 3.5 equiv). The mixture was stirred for 10 minutes to dissolve triacid, then cooled to Tj=10° C. To the cooled solution was added N-methylimidazole (115 mL, 12.0 equiv) via addition funnel, followed by solid TBTU (154 g, 4.0 equiv) in one portion. The jacket temperature was set to 20° C., and the reaction was allowed to warm over 1 hour. The reaction was then quenched by the slow addition of 50 mM potassium phosphate pH 6 buffer (1100 mL, 20 ml/g Triacid). DCM (1100 mL, 20 mL/g) was added, and the mixture was agitated for at least 10 minutes. The phases were separated, and the aqueous phase was extracted with additional DCM (1100 mL, 20 mL/g). The combined organic layers were washed with 50 mM pH 6 potassium phosphate buffer (2×1100 mL). The DCM solution was dried by azcotropic distillation to 20V, then replenished with fresh DCM (1100 mL, 20 mL/g). The reaction stream in 40 mL/g DCM is heated to Tj=30° C., then MTBE (2200 mL, 40 mL/g) is charged over 3 hours. The slurry is cooled to 25° C., aged no more than 4 hours, and filtered. The cake is washed with 1:1 DCM:MTBE (2×1100 mL, 5.0 mL/g TGZ), and dried under vacuum to provide TGZ as a white solid (88% yield). ¹H NMR (600 MHz, DMSO-d6) δ ppm 7.91 (m, 3H), 7.80 (d, J=9.2 Hz, 4H), 7.36 (m, 5H), 7.31 (dt, J=8.4, 2.7 Hz, 1H), 5.22 (d, J=3.4 Hz, 3H), 5.02 (s, 2H), 4.99 (m, 3H), 4.56 (d, J=8.7 Hz, 2H), 4.55 (d, J=8.4 Hz, 1H), 4.18 (td, J=8.2, 5.4 Hz, 1H), 4.03 (m, 9H), 3.94 (td, J=8.3, 5.7 Hz, 1H), 3.88 (m, 3H), 3.78 (m, 3H), 3.58 (m, 3H), 3.50 (m, 6H), 3.39 (m, 6H), 3.21 (m, 3H), 3.17 (m, 3H), 2.18 (m, 2H), 2.10 (s, 11H), 1.99 (m, 9H), 1.89 (s, 9H), 1.83 (m, 2H), 1.78 (s, 6H), 1.77 (s, 3H), 1.70 (m, 2H).

Screening experiments were performed to determine the various embodiments of reaction conditions described herein. Results of one example of a screen is shown in the below table. TBTU and HATU provided high conversion to TGZ product (>95 LCAP).

| | Coupling | | | Sum of Di-NAG + | TGZ |
|---|---|---|---|---|---|
| Entry | Reagent | Base | Mono-NAG | Des-Acyl | (LCAP) |
| 1 | TBTU | DIPEA | — | 0.37 | 96.1 |
| 2 | TBTU | NMM | — | 0.45 | 96.0 |
| 3 | TBTU | NMI | — | 0.15 | 96.4 |
| 4 | HATU | DIPEA | — | 0.33 | 95.4 |
| 5 | HATU | NMM | — | 0.45 | 95.1 |
| 6 | HATU | NMI | — | 0.19 | 95.8 |
| 7 | TCFH | DIPEA | 14.4 | 40.9 | 25.7 |
| 8 | TCFH | NMM | 3.15 | 34.5 | 39.2 |
| 9 | TCFH | NMI | — | 0.35 | 94.6 |

After two rounds of HTE screening and hit validation, TBTU in DMF provided full conversion of intermediates to TGZ with a clean reaction profile. While all bases included in the screening were effective, NMI provided the highest LCAP TGZ (96.36 LCAP) with 0.15 LCAP des-acyl. These conditions were subsequently validated on gram-scale, providing full conversion in 1 hour. The reaction was also demonstrated to perform equally well using dimethylacetamide (DMAc) as a solvent in place of DMF.

Additional Reagent equivalent screening experiments were also performed. Effects of reaction components on reaction rate are shown in the below table.

| | | | | | | | Sum di- | | |
|---|---|---|---|---|---|---|---|---|---|
| | | | | | NAG-H | di-NAG- | NAG/des- | | m/z |
| | NAG-H | TBTU | NMI | Time | guanidinium | OH | Ac | TGZ | 917 |
| Entry | eq. | eq. | eq. | (hr) | (LCAP) | (LCAP) | (LCAP) | (LCAP) | (LCAP) |
| 1 | 3.5 | 4 | 12 | 1 | 2.6 | 2.5 | 0.7 | 91.7 | 0.32 |
| 2 | 3.3 | 4 | 12 | 1 | 2.5 | 2.5 | 0.75 | 91.9 | 0.31 |
| 3 | 3.15 | 4 | 12 | 1 | 2.2 | 2.5 | 0.8 | 92.1 | 0.31 |
| 4 | 3 | 4 | 12 | 4 | 2.2 | 2.4 | 0.88 | 92.1 | 0.28 |
| 5 | 3.5 | 3.5 | 12 | 1 | 2.4 | 2.6 | 0.88 | 91.7 | 0.31 |
| 6 | 3.5 | 3 | 12 | 3 | 2.3 | 2.4 | 7.5 | 85.6 | 0.16 |
| 7 | 3.15 | 3.5 | 12 | 1 | 1.9 | 2.5 | 0.76 | 92.6 | 0.25 |
| 8 | 3.15 | 3.5 | 10 | 1 | 2.0 | 2.5 | 0.86 | 92.3 | 0.3 |
| 9 | 3.15 | 3.5 | 8 | 3 | 2.1 | 2.5 | 0.75 | 91.9 | 0.35 |
| 10 | 3.15 | 3.5 | 6 | 3 | 1.8 | 2.5 | 1.9 | 89.4 | 1.47 |

### Example 7: Step A-1

Step A-1: Amide coupling to prepare Methyl Core. An example of this step is shown below.

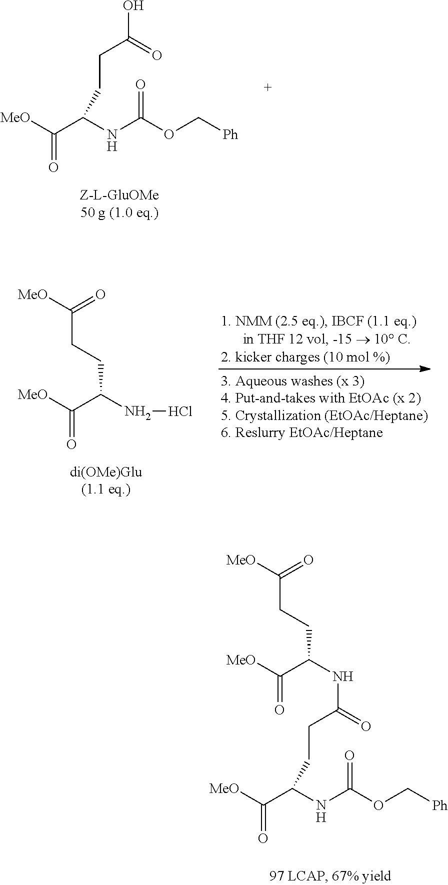 [SMILES: COC(=O)[C@H](CCC(=O)O)NC(=O)OCc1ccccc1 ; COC(=O)CC[C@H](NCl)C(=O)OC ; COC(=O)CC[C@H](NC(=O)CC[C@H](NC(=O)OCc1ccccc1)C(=O)OC)C(=O)OC]

Charged z-L-Glu-OMe (50 g, 1.0 equiv.) through a powder addition funnel into a 1 L reactor. Charged THF (10 vol) under a positive pressure of nitrogen, complete dissolution in <3 minutes. The reaction was cooled to −15° C. While the reaction was cooling down, N-Methylmorpholine (NMM) (41.8 mL, 2.5 equiv.) was charged to the reaction. Rinsed addition port with 0.5 vol THF, Once the reaction temperature reached −15° C., isobutyl chloroformate (IBCF) (22 mL, 1.1 equiv.) was charged, and white precipitate formed immediately. An internal temperature of <−10° C. was maintained during the addition. The reaction was aged for 1 h at −15° C. Charged L-Glutamic acid dimethyl ester (di(OMe)Glu) in 5 equal portions (36.2 g). Warmed reaction to 10° C. over 2 hours, aged here for 16 h. An additional 10 mol % charges of IBCF, NMM and Z-L-Glu-OMe can be added if needed. The reaction was reaction for an additional 3 h. Once reaction reached complete conversion, the reaction was carried through the work up.

Charged 6 vol of EtOAc followed by 8 vol of 0.5M HCl aqueous solution, phase cut, and drained aqueous. Charged 8 vol of 0.5M NaOH aqueous solution, phase cut, and drained aqueous. Charged 8 vol of sat. aqueous brine solution, phase cut, and drained aqueous. Drained hazy organic layer and performed a polish filtration. Added organic stream back to a clean 1 L reactor. Distilled organic solution under vacuum (45° C., 170 mmHg) down to 400 mL. Performed two sequential azeotropic distillations with EtOAc, charging an additional 300 mL (organic total volume ˜800 mL) and distilling down to −400 mL (45° C., 200 mmHg). Dropwise charged 4 vol of Heptane 40° C. Charged 2.0 wt % MethylCore seed and held at temperature for 30 mins before slowly cooling to 23° C. Charged an additional 6 volumes of heptane dropwise and let slurry age overnight.

Drained slurry into a shot bottle and filtered through a 800 mL medium porosity frit funnel. Washed cake with 2×5 vol EtOAc/Heptane (1:1). Dried cake under nitrogen/vacuum stream for 4 hr.

### Example 8: Step A-2

Step A-2: Aminolysis to prepare Triol. An example of this step is shown below.

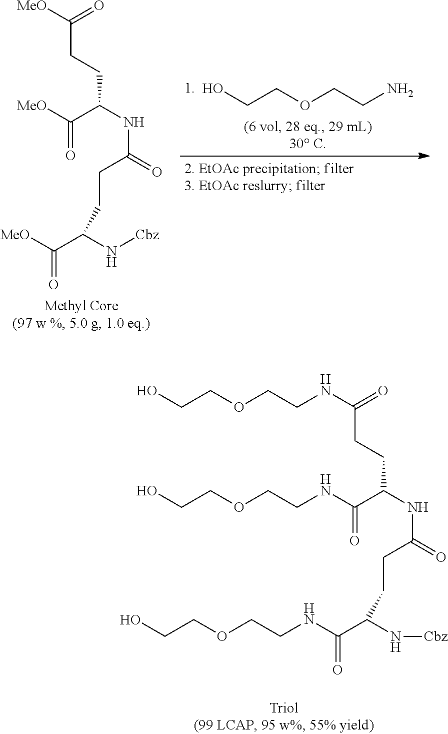

Charged Methyl Core (97 w %, 3.5 g, 1.0 equiv.) to a 100 mL reactor. Charged 2-(2-aminoethoxy)ethanol (6 vol, 21 mL) to the flask under a positive pressure of nitrogen. Set temperature to 30° C. and aged at this temperature for 24 h. Charged 20 volumes of Ethyl acetate (70 mL) to the reactor flask at 30° C. Charged 2 wt % triol seed, checked that seed holds at temperature. Cooled reaction to 23° C. and let slurry age for 18 h. Filtered slurry in a 300 mL filter funnel keeping the cake under a steady stream of nitrogen. Washed cake with 10 vol Ethyl acetate (EtOAc). Collected triol cake and added back to a clean and dried 100 mL reactor for a reslurry. Charged 15 volumes of EtOAc and warmed slurry up to 40° C.; held for 3 h; cooled back down to 23° C. and age for an additional 3 h. Filtered slurry through a 500 mLfilter funnel. Washed cake with 10 vol EtOAc and dried under nitrogen/vacuum bag for 24 h. Isolated Triol cake was 2.75 g, 55% yield.

Step A-2: Initial hydrolase screening: Preliminary enzyme screen based on the hydrolases was conducted using 10 different lipases, one peptidase, one protease, and one aldolase. The reactions were set up on a 50 mg scale with respect to Methyl Core as the limiting reagent. The amount of enzyme charged was based on the reagent physical state: liquid-based enzymes were charged in 5 L, lyophilized enzymes were charged in 5 mg and the solid-supported enzymes were charged in 50 mg, regardless of unit count. These enzymes were screened against three different solvent systems: MeCN and THF (at 20 V) and neat in the amino alcohol (8 eq.). Enzyme screen reaction conditions are shown below.

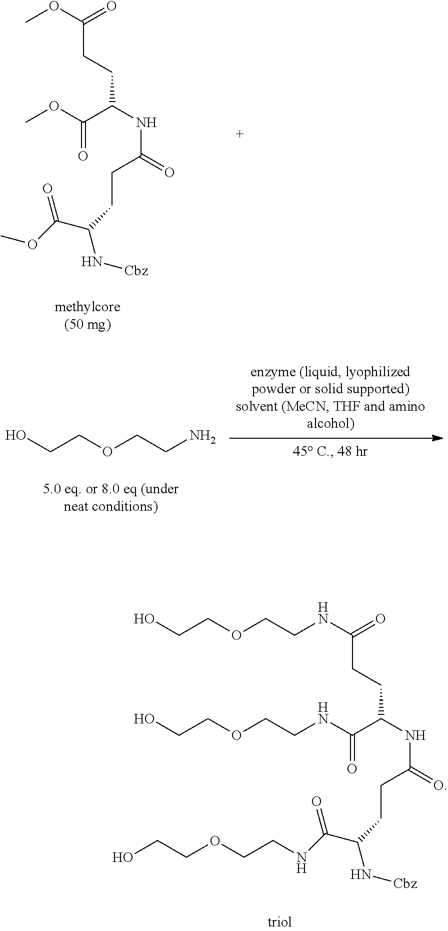

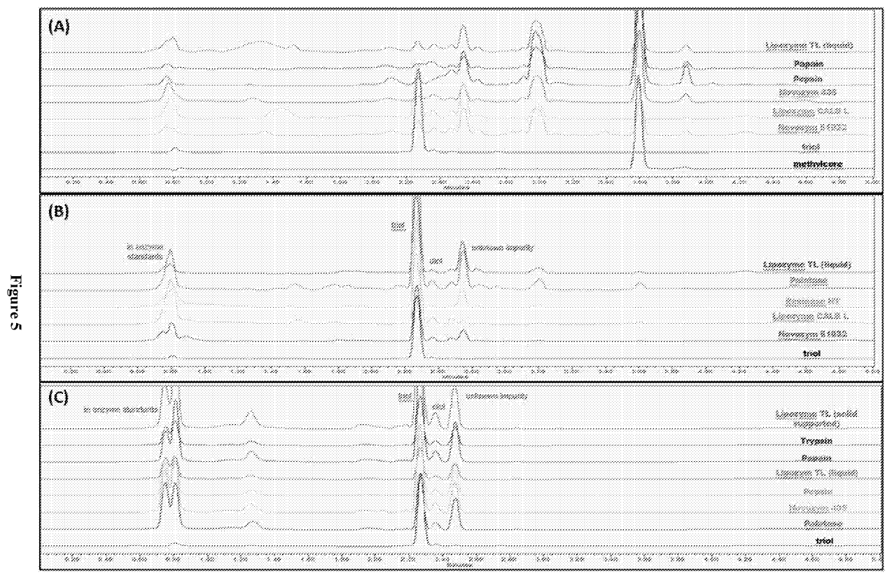

The reactions were set up in 2 mL vials and agitated on a shaker plate at 45° C. for 48 hr. The crude reaction mixtures were then analyzed by HPLC, no subsequent workups or isolations were performed. The enzyme reactivity was significantly influenced by the reaction medium, in general, the reaction outcome followed the trend: neat conditions >THF >MeCN. For all reactions conducted in the neat conditions, Triol was the major crude reaction component and Methyl Core and intermediate mono- and di-addition products were completely consumed, albeit this could be an inherent result from high reaction concentrations and higher equivalent loadings of the amino alcohol. Alternatively, MeCN was identified as a poor solvent for the biocatalytic aminolysis as most reactions resulted in a complex mixture along with unreacted starting material. The reactions in THF showed productive conversion of starting material and intermediates but the extent of conversion varied between enzymes (FIG. 5). It should be noted that some enzymes, specifically the liquid-based varients, performed better in THF, whereas the solid supported enzymes gave optimal results in the neat conditions (Table 3). An advantage to these immobilized enzymes is that they negate the need for an aqueous work up. The triol product is water soluble and if an aqueous wash is required to remove the enzyme residue from the organic product we risk losing the desired triol to the same aqueous phase. An enzyme immbolized on a solid support would allow the reagent to be filtered from the organic reaction solvent and subsequently recycled, providing minimal enzyme degradation. Additionally, once the enzyme reagent is removed from the reaction mixture, triol can be isolated by precipitation from most organic solvents including the neat reaction conditions. Of the most successful reactions between the THF and neat conditions, triol was obtained on the higher end of the 50-65% LCAP range with Novozym 51032, Lipozyme CALB L, Resinase HT, Novozym 435 and Lypozyme TL (both solid supported and liquid-based) emerging as proficient enzymes for the transformation (Table 3.) These results indicate that lipases may be the most suitable sub-class of hydrolases for the desired reaction. The diol impurity was formed in generally <10% liquid chromatography area percent (LCAP) for all reactions, supporting our hypothesis that lower reaction temperatures could deter diol formation. The main impurity formed in all reactions (15-30% LCAP) was not isolated or fully identified, by LCMS analysis of the unknown impurity possesses the same mass as the triol product and is postulated to be a stereoisomer of triol.

TABLE 3
General results from the THF- and neat-conditions enzyme screen:

| | LCAP of triol | | |
|---|---|---|---|
| Solvent | (55-65%) | LCAP of triol 45-55% | LCAP of triol <45% |
| THF | Novozym 5103 (liquid), | Novozym 435 (solid | Aldolase (lyophilized), |
| | Lipozyme CALB L | supp.), Novozym 40086 | Papain (lyophilized), |
| | (liquid), Resinase HT | (solid supp.), Pepsin | Amano PS (powder), |
| | (liquid), Lipozyme TL | (lyophilized), Lipozyme | Trypsin (lyophilized) |
| | (liquid) | TL (solid supp.), Palatase | |
| | | (liquid) | |
| Neat in | Novozym 435 (solid | Amano PS (powder), | Palatase (liquid), |
| amino | supp.), Novozym 40086 | Novozym 5103 (liquid), | aldolase (lyophilized) |
| alcohol | (solid supp.), Lipozyme | Lipozyme CALB L | |
| | TL (solid supp.), Papain | (liquid), Reinase HT | |
| | (lyophilized), Trypsin | (liquid), Lipozyme TL | |
| | (lyophilized) | (liquid) | |

These experiments demonstrated the feasibility for a biocatalyic aminolysis reaction to access Triol. Conditions were identified that allow for full conversion of starting material and intermediate mono and di-amides to give Triol as the major reaction product.

### Example 9: Step A-3

Step A-3: Triple glycosylation to prepare TGZ. An example of this step is shown below.

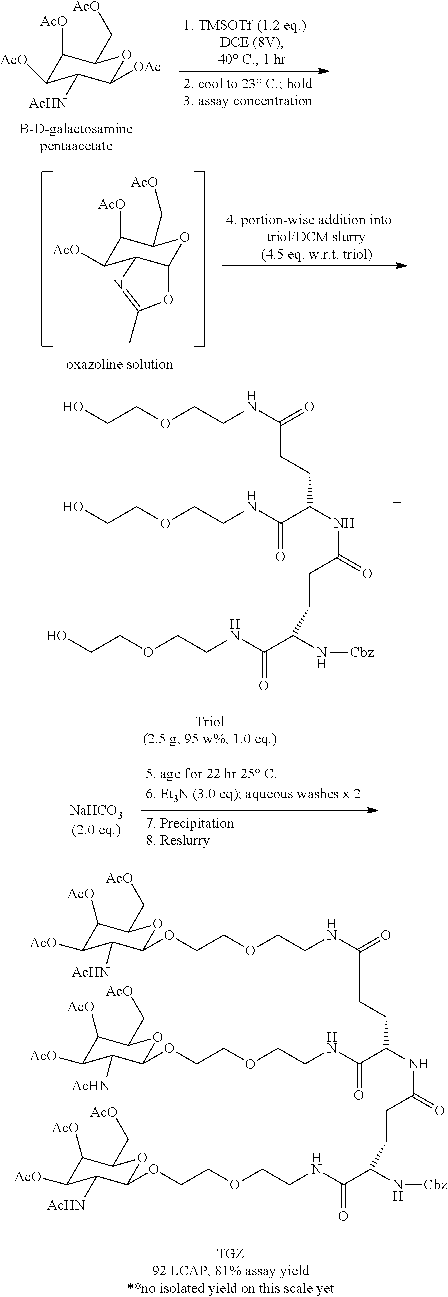

Charged Beta-D-Galactosamine pentaacetate (10.1 g) to a 100 mL reactor flask. Charged DCE (37 mL, 0.7M) to the flask under a positive pressure of nitrogen. Charged TMSOTf (6.0 mL) slowly at first for any initial vapor evolution. Warmed reaction to 40° C. for 1.5 h; then cooled back down to 23° C. In the meantime, set up reaction flask with Triol. Charged Triol (95 w %, 2.5 g, 1.0 equiv.) to a 50 mL reactor, followed by solid NaHCO₃ (0.2 equiv.). Sealed flask and inert head space with nitrogen flush. Charged DCM (6 vol) to the triol and NaHCO₃ mixture. Portion-wise charged the oxazoline solution to the triol/DCM slurry at 25° C. (1.0 eq of the oxazoline solution/hr up to 4.5 equivalents). Aged the reaction at 25° C. overnight (24 h). With high stirring, charged Et₃N (3.0 equiv.), and aged quenched reaction for 20 mins.

Charged saturated (sat.) aqueous (aq.) NaHCO₃ (10 vol); phase split, drain DCM/DCE layer, remove aqueous, recharge DCM/DCE solution; rinsed with 2 vol of DCM. Charged saturated aq. NH₄Cl (10 vol); phase split, drained DCM/DCE layer, and removed aqueous. The organic stream was dried over MgSO₄, filtered through a filter funnel; rinsed drying solids with 2 vol DCM. Charged the DCM/DCE organic solution to a clean 100 mL reactor (about 35 mL of the organic solution, 14 vol w.r.t starting material). Charged DME (17 vol, 42 mL) dropwise to organic solution over 1 hr to give a 1:1.2 ratio of solvent:anti-solvent. Aged precipitation overnight (20 h).

Filtered slurry through a medium porosity frit funnel; rinsed reactor and washed cake with 5 vol DCM/DME (1:1). Dried cake under a nitrogen/vacuum stream for 2 hours. Collected solids and added to a 100 mLreactor. Charged 20 vol (with respect to starting material) of a DCM/DME solution (1:1). Warmed slurry to 40° C.; held for 3 hr; then cooled to 25° C. and aged for an additional 2 hr. Filtered slurry through a filter funnel, washed cake with 5 vol (DCM/DME 1:1). Analyzed liquors in filtration mother liquors were 3.5 mg/mL in TGZ. TGZ cake was 92.6 LCAP. Dried cake under a nitrogen/vacuum bag to give TGZ as a solid.

### Example 10: Step 5a

An example of Step 5a is shown below.

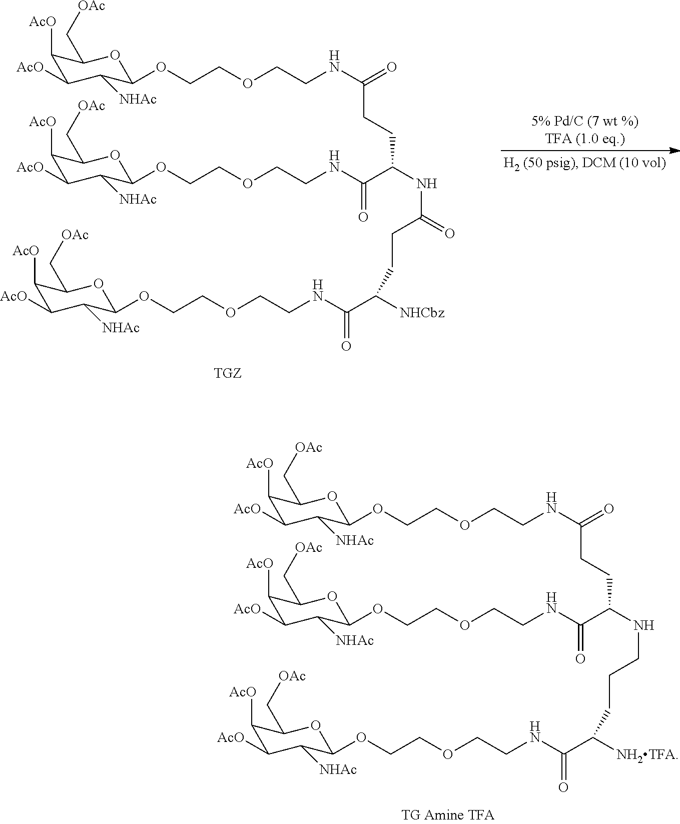

Charge TGZ (1.0 eq.) to high pressure reaction vessel, add 7% Pd/C catalyst (5 wt % loading). Charge DCM solvent (10 vol), followed by TFA (1.0 eq.). Seal reactor, conduct 3× reactor vessel purges with nitrogen gas, initiate stirring during the fill, stop stirring during the vent. Conduct 3× reactor vessel purges with H2 gas, no stirring, at 50 or 60 psi. Set reaction stir rate at 300 rpm, jacket temperature to 23° C., and H2 pressure to 45 psi. Let the reaction age overnight. Purge the headspace 3× with nitrogen gas before dismantling lid. Filter the heterogenous solution through a 0.45 micron PTE filter, rinse reactor with 3 vol DCM, add rinse to filter. Ensure Pd/C cake does not go dry. Assay the solution for potency of TG Amine, store the TG Amine-TFA solution in DCM at <10° C. TG Amine solution purity=97.2% Mass spec: Expected mass=1525.64147, Observed m/z: 1525.63930.

### Example 11: Step 5b

An example of Step 5b is shown below.

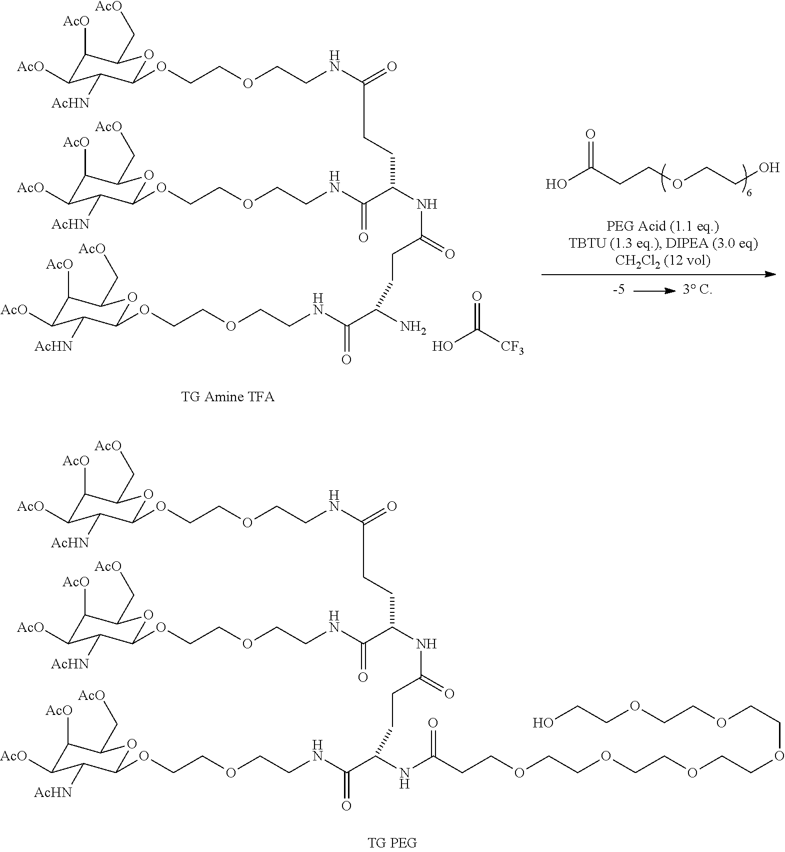

Adjust the TG Amine solution in DCM to 12 vol of DCM with respect to LC assay quantification of TG Amine (1.0 eq.). Purge reactor headspace with N2 and set reactor under N2 positive pressure, set jacket temperature to −5° C. and initiate stirring. Add PEG acid (1.1 eq.), measured by mass, followed by DIPEA (3.0 eq.). Add TBTU (1.3 eq.) solids portion-wise over 1 hour to reactor under N2 sweep. Once TBTU solids have fully dissolved (˜1 hr) set jacket temperature to ramp to 0° C. over 30 mins.

Charge 12 vol DI water to reaction and set internal temperature to 10° C. Let biphasic mixture stir for 30 mins, then let layers separate, drain DCM waste Allow reactor contents to warm to 23° C. Add 13 wt % (NH4)2SO4 (relative to water charge) to the aqueous TG PEG solution, followed by 10 vol of DCM. Mix layers over 30 mins and then allow layers to separate. Separate the layers, and wash the organic layer with a mixture of 0.5M sodium monophosphate pH 6.0 and saturated brine (4.8 vol: 3.2 vol). Allow layers to mix for 30 mins then separate layers. Wash the organic layer with a mixture of saturated sodium bicarbonate and saturated brine solution (4.0 vol: 4.0 vol). Allow layers to mix for 30 mins and then separate layers.

Charge the TG PEG/DCM solution to the reactor and distill to 5 vol. Add an additional 10 vol of fresh DCM and distill a second time to 5 vol.

Drain the concentration TG PEG/DCM solution, rinse the reactor with 0.5 vol DCM. Add 20 vol of MTBE to the reactor and set the internal temperature to 0° C. Add the TG PEG/DCM solution to the cold MTBE over 1 hour. Once the addition is finished filter the mixture. Wash the filter cake with 3 vol MTBE (×2). Then dry the solids over a nitrogen sweep for at least 24 hours.

TG PEG isolated yield=79.8% potency adjusted. HRMS: Exact mass=1861.81989, Observed m/z: 931.41 [M+2H]+

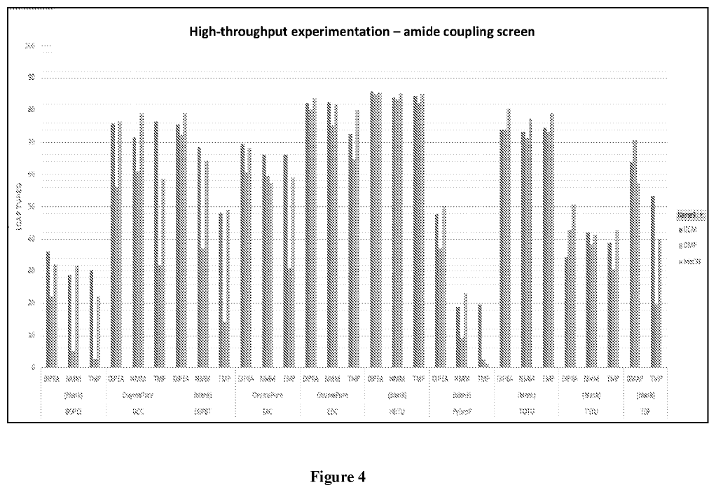

High-throughput experimentation for reaction conditions were performed using a 96-well plate. Example of conditions for screening were as follows: 1.0 equiv TG Amine TFA salt, 1.1 equiv PEG acid, 3.0 equiv base, and 1.1 equiv coupling reagent. Each combination was evaluated in DCM, DMF, and MeCN (see results in FIG. 4). The bases evaluated were DIPEA, NMI, NMM, and TMP. The results were evaluated based on LCAP of TG PEG. From this screen, HATU and TBTU provided TG PEG in >75 LCAP with full conversion of starting materials and intermediates. Order of addition experiments were also conducted wherein each of the components (DIPEA, PEG acid, TG Amine TFA solution, TBTU) were added last and the percent of impurities (e.g., des-acyl or TG PEG dimer) were measured by LC. Addition of TBTU provided the least amount of overall impurities (e.g., reduction in TG PEG dimer) and highest amount of TG PEG yield.

### Example 12: Step 6

An example of Step 6 is shown below.

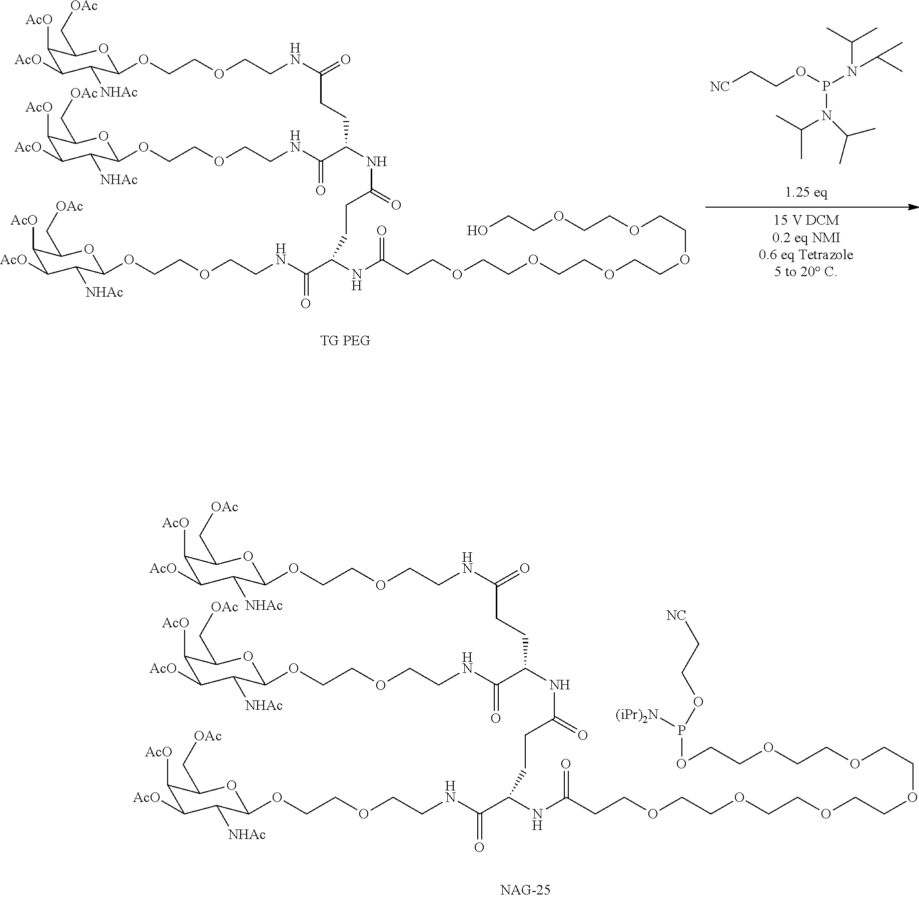

NAG-25 was prepared from TG PEG using a process description similar to the scheme above, which is further described in the steps below. HRMS: Expected mass=2078.95400, Observed m/z: 2078.95428 [M+NH4]+

To a reactor was added TG PEG (1.0 eq), DCM (20V) followed by NMI (0.2 eq) and distilled to achieve ≤200 ppm water. The reaction volume is then reduced to 15V and then cooled to 0-5° C. prior to addition of tetrazole (0.6 eq) and the P-Reagent 2-cyanoethyl-N, N, N′, N′-tetraisopropylphosphorodiamidite (1.25 eq). Upon completion of addition at 0-5° C. the reaction is then warmed to 20±5° C. and aged for 2.5 h before sampling for conversion of the starting TG PEG. Upon completion, the reaction is cooled to 10° C. then washed with 10V DI water twice. The organic solution is dried over molecular sieves and filtered under nitrogen protection. The resulting filtrate is concentrated to 8V at 20° C. then precipitated into 30V of heptane via inverse addition. The obtained solids are isolated by filtration and washed with 10 V heptane twice, followed by vacuum drying under nitrogen.

An alternative example for preparing NAG-25 from TG PEG is as follows. A solution of TG PEG (24.87 g, 13.36 mmol, 1 equiv.) was prepared in anhydrous DCM (186.5 mL, 0.07 M). Another solution containing P-Reagent 2-cyanoethyl-N, N, N′, N′-tetraisopropylphosphorodiamidite (5.57 g, 16.70 mmol, 1.25 equiv.) and 4,5-dicyanoimidazole (0.039 g, 0.33 mmol, 0.025 equiv.) was prepared in anhydrous DCM (186.5 mL, 0.07 M). The TG PEG solution was charged over 5 hours to the P-Reagent/DCI solution and was allowed to react for at least 10 hours at 40° C. After the reaction was completed, three aqueous extractions with DI water (3×248.7 mL) were performed. Then, the NAG-25 solution was azeotropically dried at 30° C. under vacuum. Following filtration of the solution, the NAG-25 solution was added to anhydrous heptane (745 mL) over 90 minutes. The solids were filtered, washed twice with anhydrous 4:1 heptane:DCM (198.9 mL heptane/49.7 mL DCM), and dried overnight under vacuum/N₂.

Experiments were conducted for reaction conditions, including activators and amounts thereof, P-reagent and amounts thereof, solvent volumes, reaction times, order of addition, and rate of addition. The reaction samples were taken for liquid chromatography measurements at various time points and LCAP were determined for TG PEG, H-Phos, NAG-25 Dimer, NAG-25, and other impurities. LC results indicated that the addition rate of TG PEG to P-reagent/DCI over a time period of of 2.5-10 hours was useful for decreasing the NAG-25 dimer formation. For mode of addition experiments, results showed that TG PEG addition to P-reagent generally reduced NAG-25 Dimer impurity. Additionally, 1.25 eq of P-reagent increased NAG-25 conversion. Results also showed a general decrease of H-PHOS after precipitation.

While examples of certain particular embodiments are provided herein, it will be apparent to those skilled in the art that various changes and modifications may be made. Such modifications are also intended to fall within the scope of the appended claims.

The foregoing description is given for clearness of understanding only, and no unnecessary limitations should be understood therefrom, as modifications within the scope of the invention may be apparent to those having ordinary skill in the art.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

1. A process of preparing NAG-25: 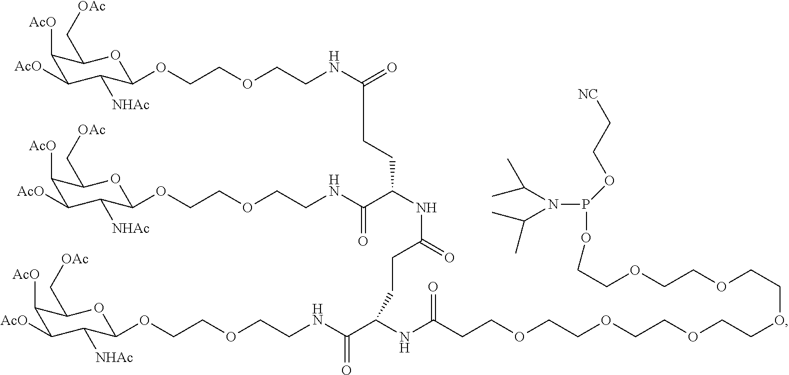 [SMILES: CC(=O)NC1C(OCCOCCNC(=O)CC[C@H](NC(=O)CC[C@H](NC(=O)CCOCCOCCOCCOCCOCCOCCOP(OCCC#N)N(C(C)C)C(C)C)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]
comprising reacting TG PEG: 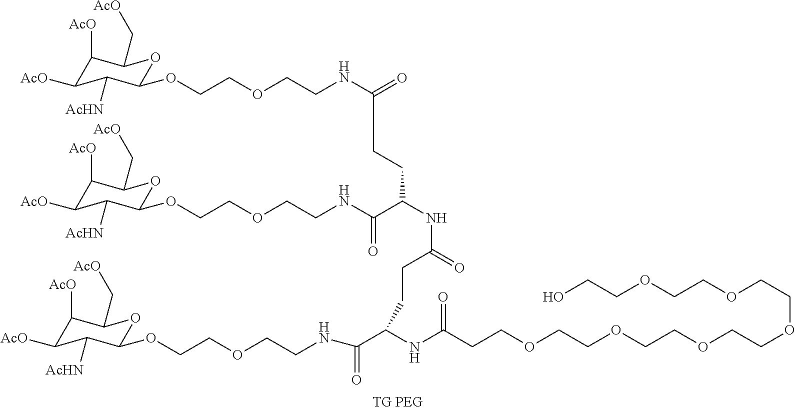
with an activator and a phosphitylating reagent, wherein the activator is tetrazole, 4,5-dicyanoimidazole (DCI), 5-Ethylthio-1H-Tetrazole (ETT), or Benzothiotetrazole (BTT); and/or wherein the phosphitylating reagent is 2-cyanoethyl-N, N, N′, N′-tetraisopropylphosphorodiamidite or 2-Cyanoethyl N,N-diisopropylchlorophosphoramidite.

2. The process of claim 1 wherein the process is performed in the presence of a base, optionally wherein the base is N-methylimidazole (NMI).

3. The process of claim 1 or 2 wherein:
a) the TG PEG is in a solution and wherein an activator and a phosphitylating reagent are combined in a another solution; and
b) the solution of TG PEG is added to the solution of activator and P-reagent over a period of time, optionally wherein the period of time is about 2.5-10 hour or optionally wherein the solution of TG PEG is allowed to react with the solution of P-reagent and activator at about 35-45° C.

4. The process of any one of claims 1-3 wherein NAG-25 is precipitated using inverse precipitation into antisolvent.

5. The process of any one of claims 1-4, where precipitation comprises precipitation of NAG-25 from dichloromethane (DCM)/heptane as solvent/antisolvent system.

6. A process for preparing TG PEG: 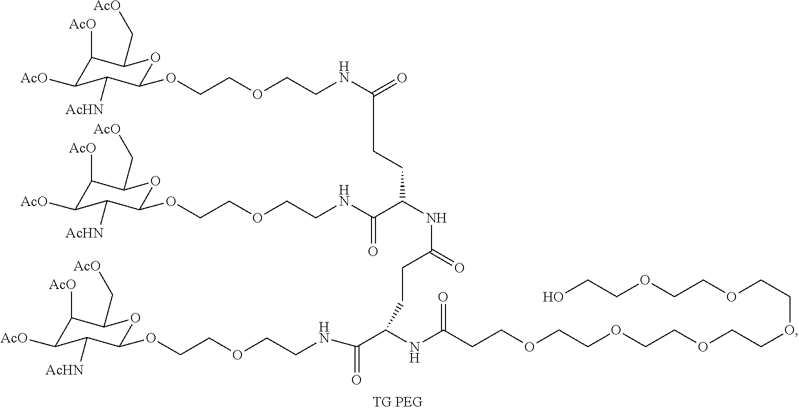
said process comprising reacting a solution of TG Amine: 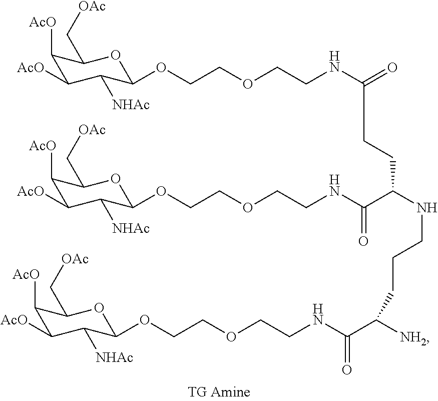 [SMILES: CC(=O)NC1C(OCCOCCNC(=O)CC[C@H](NCCC[C@H](N)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]
or a salt thereof, and PEG acid with a coupling reagent, wherein the coupling reagent is added to a solution of PEG acid and TG Amine or salt thereof over a period of time of about 30 min to about 1.5 hours.

7. The process of claim 6 wherein solution further comprises a base, optionally wherein the base is N,N-Diisopropylethylamine (DIPEA).

8. The process of any one of claims 6-7 wherein the coupling reagent is 2-(1H-Benzotriazole-1-yl)-1,1,3,3-tetramethylaminium tetrafluoroborate (TBTU) or N-[(Dimethylamino)-1H-1,2,3-triazolo-[4,5-b]pyridin-1-ylmethylene]-N-methylmethanaminium hexafluorophosphate N-oxide (HATU).

9. The process of any one of claims 6-8 wherein the TG PEG is produced in greater than or equal to about 90% purity as measured by LC or wherein TG PEG dimer impurity is present at the end of reaction at an amount of less than 10% as measured by LC.

10. The process of any one of claims 6-9 wherein the solution of TG Amine or a salt thereof is carried forward from a separate process without isolation of the TG Amine or salt thereof, optionally wherein the TG Amine or salt thereof is TG Amine trifluoroacetate (TFA) salt.

11. The process of any one of claims 6-10 further comprising transforming the TG PEG into NAG-25.

12. A process of preparing TG Amine: 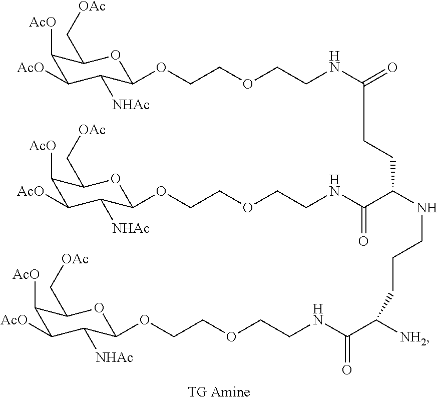 [SMILES: CC(=O)NC1C(OCCOCCNC(=O)CC[C@H](NCCC[C@H](N)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]
or a salt thereof,
said process comprising a high pressure hydrogenolysis of TGZ to form the TG Amine or a salt thereof, wherein said hydrogenolysis is performed in the presence of an acid.

13. The process of claim 12 comprising a palladium source for hydrogenolysis, optionally wherein the palladium source is palladium on carbon (Pd/C) catalyst, optionally wherein the palladium source is 5% Pd/C.

14. The process of any one of claims 12-13 wherein the acid is trifluoroacetic acid (TFA), oxalic acid, hydrochloride acid (HCl), acetic acid (AcOH), phosphoric acid (H₃PO₄), or citric acid; optionally wherein the acid is no more than about 1.1 eq.

15. The process of any one of claims 12-14 wherein said hydrogenolysis is performed in a solvent, optionally wherein the solvent is DCM, isopropyl acetate (IPAc), or methanol (MeOH).

16. The process of any one of claims 12-15 wherein the process reduces formation of one or more impurities that are TG Amine dcs-acyl, TG Amine di-NAG-OH, TG Amine guanidyl, TG Amine acyl, TG Acetamide, and/or NAG-H guanidine.

17. The process of any one of claims 12-16 wherein the TG Amine or salt thereof is carried forward as a solution of TG Amine or salt thereof and without isolation of the TG Amine or salt thereof into another process of preparing TG PEG.

18. The process of any one of claims 12-17 wherein the TG Amine or salt thereof is a TG Amine acid salt, optionally, a phosphate, formate, acetate, trifluoroacetate, or oxalate salt.

19. The process of any one of claims 12-18 further comprising transforming the TG Amine or salt thereof into NAG-25.

20. A process of preparing TGZ: 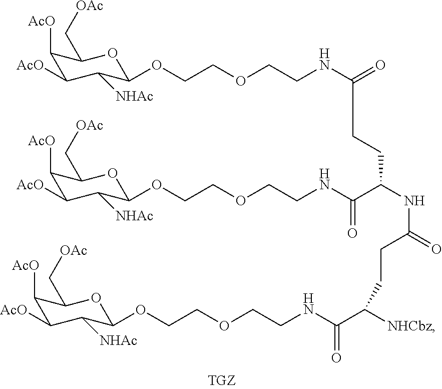 [SMILES: CC(=O)NC1C(OCCOCCNC(=O)CC[C@H](NC(=O)CC[C@H](NC(=O)OCc2ccccc2)C(=O)NCCOCCOC2OC(COC(C)=O)C(OC(C)=O)C(OC(C)=O)C2NC(C)=O)C(=O)NCCOCCOC2OC(COC(C)=O)C(OC(C)=O)C(OC(C)=O)C2NC(C)=O)OC(COC(C)=O)C(OC(C)=O)C1OC(C)=O]
said process comprising reacting Triacid with NAG-H: 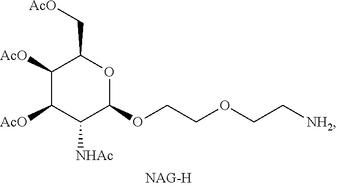 [SMILES: CC(=O)N[C@H]1[C@H](OCCOCCN)O[C@H](COC(C)=O)[C@H](OC(C)=O)[C@@H]1OC(C)=O]
or a salt thereof, in the presence of a coupling reagent and a base; precipitating TGZ from a solvent/antisolvent system.

21. The process of claim 20 wherein the coupling reagent is TBTU, HATU or Chloro-N,N,N′,N′-tetramethylformamidinium hexafluorophosphate (TCFH), optionally wherein the coupling reagent is TBTU.

22. The process of any one of claims 20-21 wherein the base is diisopropylethylamine (DIPEA), N-methylimidazole (NMI), N-methylmorpholine (NMM), or 2,2,6,6-tetramthylpiperidine (TMP), optionally wherein the base is NMI.

23. The process of any one of claims 20-22 further comprising at least one buffer wash, optionally wherein the buffer wash is about pH-5-7.

24. The process of any one of claims 20-23 wherein said reacting is conducted in a solvent selected from DCM, DMF, MeCN, and DMAc, or combination thereof.

25. The process of any one of claims 20-24 wherein an anti-solvent is added to a solution comprising TGZ and the solvent.

26. The process of claims any one of claims 24-25 wherein the anti-solvent is an ethereal solvent, optionally wherein the ethereal solvent is DME, 2-MeTHF, or MTBE.

27. The process of any one of claims 20-26 wherein TGZ is prepared with reduced levels of one or more impurities, wherein the impurities is mono-NAG, DI-NAG, DI-NAG-AZLACTONE, des-acyl, di-NAG-OH, di-NAG-OAc, NAG-H guanidine, or NAG-H trifluoroacetamide.

28. The process of any one of claims 20-27 wherein TGZ is obtained in at least 95% purity without use of column chromatography.

29. The process of any one of claims 20-28 wherein the NAG-H, or salt thereof, is a solution carried forward from another process without isolation of the NAG-H, or salt thereof.

30. The process of any one of claims 20-29 further comprising transforming the TGZ into NAG-25.

31. A process of preparing NAG-H, or salt thereof, comprising hydrogenation of NAG-Z: 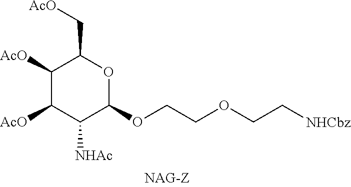 [SMILES: CC(=O)N[C@H]1[C@H](OCCOCCNC(=O)OCc2ccccc2)O[C@H](COC(C)=O)[C@H](OC(C)=O)[C@@H]1OC(C)=O]
in the presence of a Pd/C catalyst and solvent, optionally wherein the solvent is dimethylacetamide (DMAc) or DCM.

32. The process of claim 31 wherein the DMAc or DCM is about 3 volumes to about 6 volumes.

33. The process of any one of claims 31-32, further comprising acid, optionally wherein the acid is trifluoroacetic acid (TFA), acetic acid (AcOH), or Pivalic acid (PivOH), optionally wherein the acid is present at 0.01-0.04 equivalents, relative to Alcohol-Z.

34. The process of any one of claims 31-33 wherein the NAG-H or salt thereof is NAG-H TFA:

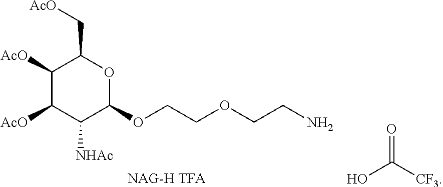 [SMILES: CC(=O)N[C@H]1[C@H](OCCOCCN)O[C@H](COC(C)=O)[C@H](OC(C)=O)[C@@H]1OC(C)=O ; O=C(O)C(F)(F)F]

35. The process of any one of claims 31-34 wherein the Pd/C catalyst is 5-10% Pd/C; optionally wherein the Pd/C is 5% Pd/C

36. The process of any one of claims 31-35, further comprising transforming the NAG-H or salt thereof into NAG-25.

37. A process for preparing NAG-Z: 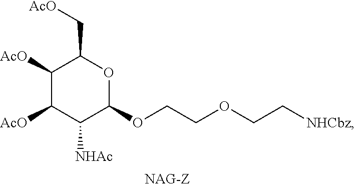 [SMILES: CC(=O)N[C@H]1[C@H](OCCOCCNC(=O)OCc2ccccc2)O[C@H](COC(C)=O)[C@H](OC(C)=O)[C@@H]1OC(C)=O]
said process comprising reacting Acyl GalNAc: 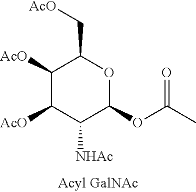 [SMILES: CC(=O)N[C@H]1[C@H](OC(C)=O)O[C@H](COC(C)=O)[C@H](OC(C)=O)[C@@H]1OC(C)=O]
with Alcohol-Z: 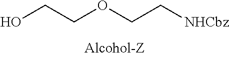 [SMILES: O=C(NCCOCCO)OCc1ccccc1]
to produce NAG-Z, wherein the reaction is performed in the presence of acid.

38. The process of claim 37 wherein the acid is bismuth trifluoromethanesulfonate (Bi(OTf)₃), boron trifluoride etherate (BF₃OEt₂), tert-butyldimethylsilyl trifluoromethanesulfonate (TBSOTf), triisopropylsilyl trifluoromethanesulfonate (TIPSOTf), indium triflate (In(OTf)₃), or copper triflate (Cu(OTf)₂).

39. The process of any one of claims 37-38 wherein the reaction is performed in the presence of a solvcnt selected from acetonitrile, dichloromethane, and dichloroethane.

40. The process of any one of claims 37-39 wherein the: Acyl GalNAc is present at about 1.0-2.0 equivalents, relative to Alcohol-Z; or acid is present at about 0.01-0.1 equivalents, relative to Alcohol-Z.

41. The process of any one of claims 37-40 wherein the reaction temperature is heated to about 55-65° C. or 58-62° C.

42. The process of any one of claims 37-41 wherein the solvent is acetonitrile and antisolvent is added is added to crystalline the NAG-Z, optionally wherein the antisolvent is water or MTBE.

43. The process of any one of claims 37-42 further comprising transforming the NAG-Z into NAG-25.

44. A process of preparing Alcohol-Z, said process comprising reacting benzyl chloroformate with aminoalcohol in presence of a solvent and a base.

45. The process of claim 43-44 wherein the solvent is dichloromethane (DCM) and the base is triethylamine (TEA).

46. The process of any one of claims 43-45 wherein the aminoalcohol is present at 1.1 equivalents, relative to benzyl chloroformate and/or the reaction temperature is about 30-40° C.

47. A crystalline Form I of Triacid, characterized by an X-ray powder diffractogram having at least a signal at three two-theta values selected from 7.4±0.2, 9.2±0.2, 21.0±0.2, 14.6±0.2, 18.3±0.2, and 19.3±0.2 or characterized by an X-ray powder diffractogram substantially similar to that in FIG. 2.

48. A crystalline Form II of Triacid, characterized by an X-ray powder diffractogram having at least a signal at three two-theta values selected from 7.8±0.2, 9.7±0.2, 13.3±0.2, 15.7±0.2, 17.1±0.2, 18.9±0.2, 20.2±0.2, and 21.0±0.2 or characterized by an X-ray powder diffractogram substantially similar to that in FIG. 3.

49. A process of preparing Triacid: 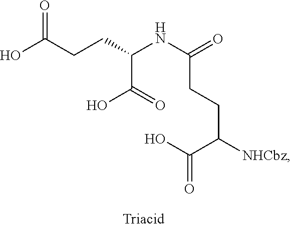 [SMILES: O=C(O)CC[C@H](NC(=O)CCC(NC(=O)OCc1ccccc1)C(=O)O)C(=O)O]
said process comprising reacting t-Butyl core with an acid, wherein the acid is phosphoric acid (H₃PO₄), TFA, HCl, benzenesulfonic acid, or p-toluenesulfonic acid.

50. The process of claim 49 wherein the reaction is conducted in a solvent selected from 2-MeTHF, acetonitrile, THF, DMA, sulfolane, and DME.

51. The process of any one of claims 49-50 wherein the solvent is mixed with water, optionally wherein the solvent is 2-MeTHF.

52. The process of any one of claims 49-51 wherein the solvent is about 3.0-4.0 vol of 2-MeTHF and is mixed with about 0.5-2 vol water.

53. The process of any one of claims 49-52 wherein reaction is conducted at a temperature of about 40-60° C.

54. The process of any one of claims 49-53 wherein the Triacid is isolated as a crystalline solid.

55. The process of any one of claims 49-54 further comprising crystallization of Triacid using acetone/toluene, 2-MeTHF/IPAc, 2-MeTHF/CPME, acetone/heptane, or MeTHF/acetonitrile.

56. The process of any one of claims 49-55 wherein the Triacid is obtained in a crystalline form characterized by an X-ray powder diffractogram having at least a signal at three two-theta values chosen from 7.4±0.2, 9.2±0.2, 21.0±0.2, 14.6±0.2, 18.3±0.2, and 19.3±0.2 or characterized by an X-ray powder diffractogram having at least a signal at three two-theta values selected from 7.8±0.2, 9.7±0.2, 13.3±0.2, 15.7±0.2, 17.1±0.2, 18.9±0.2, 20.2±0.2, and 21.0±0.2.

57. The process of any one of claims 49-56 further comprising transforming the Triacid into NAG-25.

58. A process for preparing t-Butyl Core: 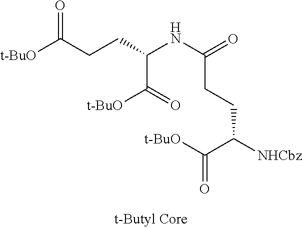 [SMILES: CC(C)(C)OC(=O)CC[C@H](NC(=O)CC[C@H](NC(=O)OCc1ccccc1)C(=O)OC(C)(C)C)C(=O)OC(C)(C)C]
said process comprising reacting GluZ: 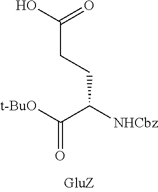 [SMILES: CC(C)(C)OC(=O)[C@H](CCC(=O)O)NC(=O)OCc1ccccc1]
with GluOtBu or salt thereof: 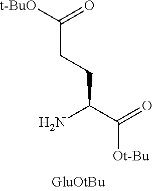 [SMILES: CC(C)(C)OC(=O)CC[C@H](N)C(=O)OC(C)(C)C]
to produce t-Butyl Core, wherein the reacting is performed in the presence of a coupling reagent, a base, and a solvent; optionally wherein the a) coupling reagent is EDC/Oxyma, TFFH, PyOxim, CDI, PivCl, T3P, or COMU; or b) base is N-methylmorpholine (NMM); and/or c) solvent is IPAc, MeTHF, MIBK, or MTBE.

59. The process of claim 58 wherein GluOtBu or salt thereof is a GluOtBu hydrochloride salt:

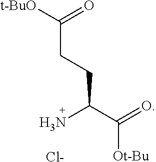 [SMILES: CC(C)(C)OC(=O)CC[C@H](N)C(=O)OC(C)(C)C ; [Cl-]]

60. The process of claim 58 or 59

61. The process of any one of claims 58-60 wherein the:
coupling reagent is PivCl, solvent is MTBE; optionally wherein the GluOtBu is about 1-1.5 eq.; or PivCl is about 1-1.5 eq; or NMM is about 3-4 eq.; or MTBE is about 8-12 vol, each relative to GluZ.

62. The process of any one of claims 58-61 wherein a first solution comprising GluZ and NMM in a solvent is added to a second solution comprising the solvent and coupling reagent; optionally wherein the coupling reagent is in excess and/or GluOtBu is in excess, optionally wherein the coupling reagent is PivCl

63. The process of any one of claims 58-62 further comprising addition of an antisolvent, optionally over a time period of about 2-5 hours.

64. The process of any one of claims 58-63 further comprising transforming the t-Butyl Core into NAG-25.

65. A process for preparing Methyl Core: 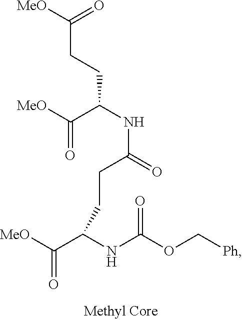 [SMILES: COC(=O)CC[C@H](NC(=O)CC[C@H](NC(=O)OCc1ccccc1)C(=O)OC)C(=O)OC]
said process comprising reacting z-L-Glu-OMe: 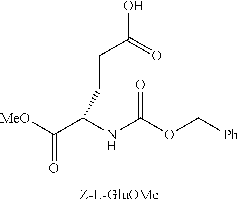 [SMILES: COC(=O)[C@H](CCC(=O)O)NC(=O)OCc1ccccc1]
with L-Glutamic acid dimethyl ester (di(OMe)Glu): 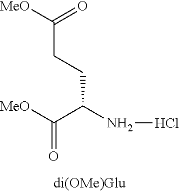 [SMILES: COC(=O)CC[C@H](NCl)C(=O)OC]
to produce Methyl Core, optionally in the presence of a base and a coupling reagent.

66. The process of claim 65, wherein: a) the base is added to a z-L-GluOMe solution prior to addition of the coupling reagent and di(OMe)Glu to the reaction; or b) wherein the coupling reagent is added to the solution of z-L-Glu-OMe and base prior to addition of di(OMe)Glu to the reaction.

67. The process of any one of claims 65-66 further comprising a solvent, optionally wherein the solvent is THF or MeTHF.

68. The process of any one of claims 65-67 wherein the base is N-Methylmorpholine (NMM) or di-isopropylethylamine and/or the coupling reagent is isobutyl chloroformate (TBCF), TBTU, HATU, EDC, or DCC.

69. The process of any one of claims 65-68 further comprising transforming the Methyl Core into NAG-25.

70. A compound: 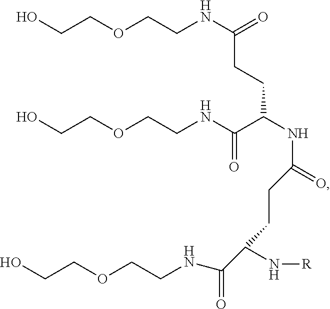
wherein R is H (unprotected Triol) or a Cbz protecting group (Triol).

71. A process for preparing Triol: 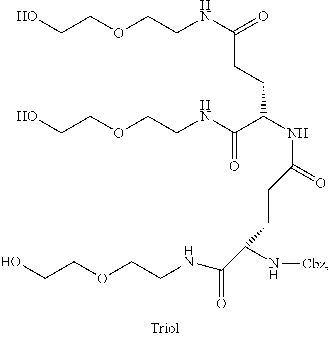
the process comprising reacting Methyl Core with 2-(2-aminoethoxy)ethanol: 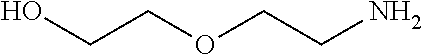
to produce Triol, optionally wherein the Triol is transformed into NAG-25.

72. A process for preparing TGZ, the process comprising reacting protected Triol: 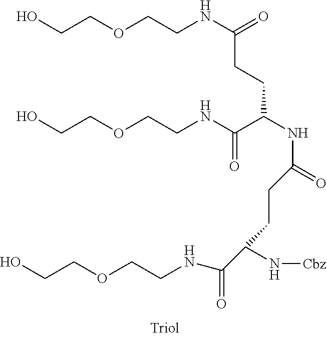
with Beta-D-Galactosamine pentaacetate: 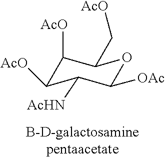 [SMILES: CC(=O)NC1C(OC(C)=O)OC(COC(C)=O)C(OC(C)=O)C1OC(C)=O]
pentaacetate to produce TGZ, optionally wherein the TGZ is transformed into NAG-25.
